SEQUENCE LISTING (380 sequences)

SEQ ID NO: 1 - protein, 123 aa, Artificial Sequence
  Synthetic
EVQLVESGGGLVQPGGSLRLSCAASGFTFSSYWMSWVRQAPGKGLEWVSIINYSGREADYADSVKGRFTISRDNSKNTLYLQMNSLRAEDTAVYYCARDGSYVGQAHEAFDIWGQGTLVTVSS

SEQ ID NO: 2 - protein, 123 aa, Artificial Sequence; identical to SEQ ID NO: 1
  Synthetic

SEQ ID NO: 3 - protein, 120 aa, Artificial Sequence
  Synthetic
EVQLVESGGGLVQPGGSLRLSCAASGFTFSSAWMSWVRQAPGKGLEWVSTIYWSGSEINYADSVKGRFTISRDNSKNTLYLQMNSLRAEDTAVYYCARSFASGESAMDVWGQGTLVTVSS

SEQ ID NO: 4 - protein, 107 aa, Artificial Sequence
  Synthetic
DIQMTQSPSSLSASVGDRVTITCRASQSISNYLAWYQQKPGKAPKLLIYDASNLQSGVPSRFSGSGSGTDFTLTISSLQPEDFATYYCQQYYSSPPTFGQGTKVEIK

SEQ ID NO: 5 - protein, 116 aa, Artificial Sequence
  Synthetic
EVQLVESGGGLVQPGGSLRLSCAASGFTFSTSWMHWVRQAPGKGLEWVSIINSGGGEAYYADSVKGRFTISRDNSKNTLYLQMNSLRAEDTAVYYCARGDAAFDYWGQGTLVTVSS

SEQ ID NO: 6 - protein, 107 aa, Artificial Sequence
  Synthetic
DIQMTQSPSSLSASVGDRVTITCRASQLIRKKLAWYQQKPGKAPKLLIYAASTLQSGVPSRFSGSGSGTDFTLTISSLQPEDFATYYCMQSGSPQYTFGQGTKVEIK

SEQ ID NO: 7 - protein, 120 aa, Artificial Sequence
  Synthetic
EVQLVESGGGLVQPGGSLRLSCAASGFTFTNYWMGWVRQAPGKGLEWVSIIISDGGEIIYADSVKGRFTISRDNSKNTLYLQMNSLRAEDTAVYYCARIHAGTGSSADYWGQGTLVTVSS

SEQ ID NO: 8 - protein, 107 aa, Artificial Sequence
  Synthetic
DIQMTQSPSSLSASVGDRVTITCRASQSIYNYLNWYQQKPGKAPKLLIYDASTLQSGVPSRFSGSGSGTDFTLTISSLQPEDFATYYCQQYYSYPWTFGQGTKVEIK

SEQ ID NO: 9 - protein, 107 aa, Artificial Sequence; identical to SEQ ID NO: 8
  Synthetic

SEQ ID NO: 10 - protein, 106 aa, Artificial Sequence
  Synthetic
DIQMTQSPSSLSASVGDRVTITCRASQSIYSYLNWYQQKPGKAPKLLIYGASILQYGVPSRFSGSGSGTDFTLTISSLQPEDFATYYCHQSNSSPGTFGQGTKVEI

SEQ ID NO: 11 - protein, 106 aa, Artificial Sequence; identical to SEQ ID NO: 10
  Synthetic

SEQ ID NO: 12 - protein, 107 aa, Artificial Sequence
  Synthetic
DIQMTQSPSSLSASVGDRVTITCRASQGLSSYLNWYQQKPGKAPKLLIYGMSTLQSGVPSRFSGSGSGTDFTLTISSLQPEDFATYYCMQYYHWPYTFGQGTKVEIK

SEQ ID NO: 13 - protein, 124 aa, Artificial Sequence
  Synthetic
EVQLVESGGGLVQPGGSLRLSCAASGFTFSDYIMHWVRQAPGKGLEWVSAISPSGSEIYYADSVKGRFTISRDNSKNTLYLQMNSLRAEDTAVYYCARNAWDNNWVREYGMDYWGQGTLVTVSS

SEQ ID NO: 14 - protein, 107 aa, Artificial Sequence
  Synthetic
DIQMTQSPSSLSASVGDRVTITCRASQSGNNNLAWYQQKPGKAPKLLIYGASTLQSGVPSRFSGSGSGTDFTLTISSLQPEDFATYYCMQSNSYPLTFGQGTKVEIK

SEQ ID NO: 15 - protein, 107 aa, Artificial Sequence; identical to SEQ ID NO: 14
  Synthetic

SEQ ID NO: 16 - protein, 107 aa, Artificial Sequence
  Synthetic
DIQMTQSPSSLSASVGDRVTITCRASQDVDSYLNWYQQKPGKAPKLLIYDAISLQSGVPSRFSGSGSGTDFTLTISSLQPEDFATYYCQQYYSLPLTFGQGTKVEIK

SEQ ID NO: 17 - protein, 120 aa, Artificial Sequence
  Synthetic
EVQLVESGGGLVQPGGSLRLSCAASGFTFSDYAIGWVRQAPGKGLEWVSTIYWSGSNAYYADSVKGRFTISRDNSKNTLYLQMNSLRAEDTAVYYCARLFTLGYHGFDVWGQGTLVTVSS

SEQ ID NO: 18 - protein, 107 aa, Artificial Sequence
  Synthetic
DIQMTQSPSSLSASVGDRVTITCRASQSINTYLNWYQQKPGKAPKLLIYDASNLQSGVPSRFSGSGSGTDFTLTISSLQPEDFATYYCQQYDDLPFTFGQGTKVEIK

SEQ ID NO: 19 - protein, 117 aa, Artificial Sequence
  Synthetic
EVQLVESGGGLVQPGGSLRLSCAASGFTFSSNSMSWVRQAPGKGLEWVSAINYSGREIYYADSVKGRFTISRDNSKNTLYLQMNSLRAEDTAVYYCARTDYNTFDYWGQGTLVTVSS

SEQ ID NO: 20 - protein, 107 aa, Artificial Sequence
  Synthetic
DIQMTQSPSSLSASVGDRVTITCRASQSNSSHLNWYQQKPGKAPKLLIYGVSNLQSGVPSRFSGSGSGTDFTLTISSLQPEDFATYYCQHYGSTPYTFGQGTKVEIK

SEQ ID NO: 21 - protein, 120 aa, Artificial Sequence
  Synthetic
EVQLVESGGGLVQPGGSLRLSCAASGFTFSDYYMSWVRQAPGKGLEWVSVISYGGGEAYYADSVKGRFTISRDNSKNTLYLQMNSLRAEDTAVYYCARAMASEYGPWDYWGQGTLVTVSS

SEQ ID NO: 22 - protein, 107 aa, Artificial Sequence
  Synthetic
DIQMTQSPSSLSASVGDRVTITCRASQSISRHLTWYQQKPGKAPKLLIYGASTLQSGVPSRFSGSGSGTDFTLTISSLQPEDFATYYCQQYHDWPLTFGQGTKVEIK

SEQ ID NO: 23 - protein, 123 aa, Artificial Sequence
  Synthetic
EVQLVESGGGLVQPGGSLRLSCAASGFTFGDDYMHWVRQAPGKGLEWVSAINYDGSWKYYADSVKGRFTISRDNSKNTLYLQMNSLRAEDTAVYYCARLSSIDEPPYGPFDVWGQGTLVTVSS

SEQ ID NO: 24 - protein, 107 aa, Artificial Sequence
  Synthetic
DIQMTQSPSSLSASVGDRVTITCRASQSISTYLAWYQQKPGKAPKLLIYEASSLQSGVPSRFSGSGSGTDFTLTISSLQPEDFATYYCQQYYNFPPTFGQGTKVEIK

SEQ ID NO: 25 - protein, 121 aa, Artificial Sequence
  Synthetic
EVQLVESGGGLVQPGGSLRLSCAASGFTFNNYYMSWVRQAPGKGLEWVSIIYYDGSEADYADSVKGRFTISRDNSKNTLYLQMNSLRAEDTAVYYCARNKDIYSVYGMDYWGQGTLVTVSS

SEQ ID NO: 26 - protein, 107 aa, Artificial Sequence
  Synthetic
DIQMTQSPSSLSASVGDRVTITCRASQSISNYLAWYQQKPGKAPKLLIYATSNLQSGVPSRFSGSGSGTDFTLTISSLQPEDFATYYCQQANNTPPTFGQGTKVEIK

SEQ ID NO: 27 - protein, 120 aa, Artificial Sequence
  Synthetic
EVQLVESGGGLVQPGGSLRLSCAASGFTFSDYWMSWVRQAPGKGLEWVSSIVYGGSEIDYADSVKGRFTISRDNSKNTLYLQMNSLRAEDTAVYYCARVPGYSGTPFDYWGQGTLVTVSS

SEQ ID NO: 28 - protein, 107 aa, Artificial Sequence
  Synthetic
DIQMTQSPSSLSASVGDRVTITCRASQSVSRYLAWYQQKPGKAPKLLIYDTSSLQSGVPSRFSGSGSGTDFTLTISSLQPEDFATYYCQQSYSFPLTFGQGTKVEIK

SEQ ID NO: 29 - protein, 120 aa, Artificial Sequence
  Synthetic
EVQLVESGGGLVQPGGSLRLSCAASGFTFKDSWMHWVRQAPGKGLEWVSIISYSGGEAIYADSVKGRFTISRDNSKNTLYLQMNSLRAEDTAVYYCARDSGGSAMGFDIWGQGTLVTVSS

SEQ ID NO: 30 - protein, 107 aa, Artificial Sequence
  Synthetic
DIQMTQSPSSLSASVGDRVTITCRASQSIHSYLNWYQQKPGKAPKLLIYGASSLQSGVPSRFSGSGSGTDFTLTISSLQPEDFATYYCQQYYRFPYTFGQGTKVEIK

SEQ ID NO: 31 - protein, 120 aa, Artificial Sequence
  Synthetic
EVQLVESGGGLVQPGGSLRLSCAASGFTFSDWYLHWVRQAPGKGLEWVSVINGGGSEIIYADSVKGRFTISRDNSKNTLYLQMNSLRAEDTAVYYCARGGDGDGSGFDDWGQGTLVTVSS

SEQ ID NO: 32 - protein, 107 aa, Artificial Sequence
  Synthetic
DIQMTQSPSSLSARVGDRVTITCRASQSIHSYLNWYQQKPGKAPKMLIYDASNLQSGVPSRFSGSGSGTDFTLTISSLQPEDFATYYCQQSGNYPFTFGQGTKVEIK

SEQ ID NO: 33 - protein, 125 aa, Artificial Sequence
  Synthetic
EVQLVESGGGLVQPGGSLRLSCAASGFTFSFTAMHWVRQAPGKGLEWVSAIIYNGSEADYADSVKGRFTISRDNSKNTLYLQMNSLRAEDTAVYYCARANDYDHGCCDNYAMDYWGQGTLVTVSS

SEQ ID NO: 34 - protein, 107 aa, Artificial Sequence
  Synthetic
DIQMTQSPSSLSASVGDRVTITCRASQGIGSYLYWYQQKPGKAPKLLIYDASNLQSGVPSRFSGSGSGTDFTLTISSLQPEDFATYYCMQHGGWPLTFGQGTKVEIK

SEQ ID NO: 35 - protein, 107 aa, Artificial Sequence; identical to SEQ ID NO: 34
  Synthetic

SEQ ID NO: 36 - protein, 107 aa, Artificial Sequence
  Synthetic
DIQMTQSPSSLSASVGDRVTITCRASQSISNYLAWYQQKPGKAPKLLIYDANNLQSGVPSRFSGSGSGTDFTLTISSLQPEDFATYYCQQSYSWPLTFGQGTKVEIK

SEQ ID NO: 37 - protein, 119 aa, Artificial Sequence
  Synthetic
EVQLVESGGGLVQPGGSLRLSCAASGFTFTEWWMSWVRQAPGKGLEWVSTISYGGSEAIYADSVKGRFTISRDNSKNTLYLQMNSLRAEDTAVYYCARTSSDRLLFDYWGQGTLVTVSS

SEQ ID NO: 38 - protein, 107 aa, Artificial Sequence
  Synthetic
DIQMTQSPSSLSASVGDRVTITCRASQSIKSSLAWYQQKPGKAPKLLIYGASTLQSGVPSRFSGSGSGTDFTLTISSLQPEDFATYYCMQTNRHPWTFGQGTKVEIK

SEQ ID NO: 39 - protein, 120 aa, Artificial Sequence
  Synthetic
EVQLVESGGGLVQPGGSLRLSCAASGFTFSNYNLHWVRQAPGKGLEWVSTISYSGSEIIYADSVKGRFTISRDNSKNTLYLQMNSLRAEDTAVYYCAREDEYTYYYFDPWGQGTLVTVSS

SEQ ID NO: 40 - protein, 107 aa, Artificial Sequence
  Synthetic
DIQMTQSPSSLSASVGDRVTITCRASQSVSSYLAWYQQKPGKAPKLLIYDASKLQSGVPSRFSGSGSGTDFTLTISSLQPEDFATYYCKQSYSSPPTFGQGTKVEIK

SEQ ID NO: 41 - protein, 120 aa, Artificial Sequence
  Synthetic
EVQLVESGGGLVQPGGSLRLSCAASGFTFSDYWMHWVRQAPGKGLEWVSTIIGGGSEAGYADSVKGRFTISRDNSKNTLYLQMNSLRAEDTAVYYCARDRSYGYLGFDIWGQGTLVTVSS

SEQ ID NO: 42 - protein, 107 aa, Artificial Sequence
  Synthetic
DIQMTQSPSSLSASVGDRVTITCRASQSIRNSLHWYQQKPGKAPKLLIYSAGKLQSGVPSRFSGSGSGTDFTLTISSLQPEDFATYYCQQSNSFPLTFGQGTKVEIK

SEQ ID NO: 43 - protein, 118 aa, Artificial Sequence
  Synthetic
EVQLVESGGGLVQPGGSLRLSCAASGFTFSTNWMHWVRQAPGKGLEWVSMISYSGGEAIYADSVKGRFTISRDNSKNTLYLQMNSLRAEDTAVYYCARNWLPYAMDYWGQGTLVTVSS

SEQ ID NO: 44 - protein, 107 aa, Artificial Sequence
  Synthetic
DIQMTQSPGSLSASVGDRVTITCRASQSIRSYLSWYQQKPGKAPKLLIYSASTLQSGVPSRFSGSGSGTDFTLTISSLQPEDFATYYCQQSYSTPLTFGQGTKVEIK

SEQ ID NO: 45 - protein, 120 aa, Artificial Sequence
  Synthetic
AVQLVESGGGLVQPGGSLRLSCAASGFTFSSYDVGWVRQAPGKGLEWVSGIVPSGGNIYYADSVKGRFTISRDNSKNTLYLQMNSLRAEDTAVYYCARHRSYAYYAFDYWGQGTLVTVSS

SEQ ID NO: 46 - protein, 107 aa, Artificial Sequence
  Synthetic
DIQMTQSPSSLSASVGDRVTITCRASQNVRNYLGWYQQKPGKAPKLLIYDASSLQSGVPSRFSGSGSGTDFTLTISSLQPEDFATYYCQQYGDWPLTFGQGTKVEIK

SEQ ID NO: 47 - protein, 120 aa, Artificial Sequence
  Synthetic
EVQLVESGGGLVQPGGSLRLSCAASGFTFTDAYMSWVRQAPGKGLEWVSTISSYGSSTYYADSVKGRFTISRDNSKNTLYLQMNSLRAEDTAVYYCARPRYIESPVYDYWGQGTLVTVSS

SEQ ID NO: 48 - protein, 107 aa, Artificial Sequence
  Synthetic
DIQMTQSPSSLSASVGDRVTITCRASQSIAKYLAWYQQKPGKAPKLLIYETSNLQSGVPSRFSGSGSGTDFTLTISSLQPEDFATYYCQQSSSSPETFGQGTKVEIK

SEQ ID NO: 49 - protein, 118 aa, Artificial Sequence
  Synthetic
EVQLVESGGGLVQPGGSLRLSCAASGFTFTNYWMHWVRQAPGKGLEWVSTIIYDGGETGYADSVKGRFTISRDNSKNTLYLQMNSLRAEDTAVYYCARNSRKSGMDYWGQGTLVTVSS

SEQ ID NO: 50 - protein, 107 aa, Artificial Sequence
  Synthetic
DIQMTQSPSSLSASVGDRVTITCRASQSIYKYLNWYQQKPGKAPKLLIYDASTLQSGVPSRFSGSGSGTDFTLTISSLQPEDFATYYCMQYYSDPLTFGQGTKVEIK

SEQ ID NO: 51 - protein, 123 aa, Artificial Sequence
  Synthetic
EVQLVESGGGLVQPGGSLRLSCAASGFTFSGYAWSWVRQAPGKGLEWVSMISPAGGSTYYADSVKGRFTISRDNSKNTLYLQMNSLRAEDTAVYYCARDRNAGGDSYYSFDVWGQGTLVTVSS

SEQ ID NO: 52 - protein, 107 aa, Artificial Sequence
  Synthetic
DIQMTQSPSSLSASVGDRVTITCRASQSINSHLAWYQQKPGKAPKLLIYAAINLQSGVPSRFSGSGSGTDFTLTISSLQPEDFATYYCQQTNHYPLTFGQGTKVEIK

SEQ ID NO: 53 - protein, 121 aa, Artificial Sequence
  Synthetic
EVQLVESGGGLVQPGGSLRLSCAASGFTFSSHAMHWVRQAPGKGLEWVSSILSGGSETNYADSVKGRFTISRDNSKNTLYLQMNSLRAEDTAVYYCARDTYEVTGNLLDYWGQGTLVTVSS

SEQ ID NO: 54 - protein, 107 aa, Artificial Sequence
  Synthetic
DIQMTQSPSSLSASVGDRVTITCRASQSVWSYLNWYQQKPGKAPKLLIYGASSLQSGVPSRFSGSGSGTDFTLTISSLQPEDFATYYCMQSYSWPFTFGQGTKVEIK

SEQ ID NO: 55 - protein, 120 aa, Artificial Sequence
  Synthetic
EVQLVESGGGLVQPGGSLRLSCAASGFTFNEYAMSWVRQAPGKGLEWVSSIIPDGSETDYADSVKGRFTISRDNSKNTLYLQMNSLRAEDTAVYYCARSLSSSGMHGDIWGQGTLVTVSS

SEQ ID NO: 56 - protein, 106 aa, Artificial Sequence
  Synthetic
DIQMTQSPSSLSASVGDRVTITCRASQSINNYLNWYQQKPGKAPKLLIYKASTLQSGVPSRFSGSGSGTDFTLTISSLQPEDFATYYCHQYHSPYTFGQGTKVEIK

SEQ ID NO: 57 - protein, 127 aa, Artificial Sequence
  Synthetic
EVQLVESGGGLVQPGGSLRLSCAASGFTFSDYGWHWVRQAPGKGLEWVSIIESDGSGTYYADSVKGRFTISRDNSKNTLYLQMNSLRAEDTAVYYCARNGEVGERGVRDYDYAMDYWGQGTLVTVSS

SEQ ID NO: 58 - protein, 107 aa, Artificial Sequence
  Synthetic
DIQMTQSPRSLSASVGDRVTITCRASQSINRYLNWYQQKPGKAPKLLIYATSSLQSGVPSRFSGSGSGTDFTLTISSLQPEDFATYYCQQYGSTPITFGQGTKVEIK

SEQ ID NO: 59 - protein, 125 aa, Artificial Sequence
  Synthetic
EVQLVESGGGLVQPGGSLRLSCAASGFTFTDYNVHWVRQAPGKGLEWVSGINSSGSETNYADSVKGRFTISRDNSKNTLYLQMNSLRAEDTAVYYCARDSVFKTVGGYDAVMDYWGQGTLVTVSS

SEQ ID NO: 60 - protein, 107 aa, Artificial Sequence
  Synthetic
DIQMTQSPSSLSASVGDRVTITCRASQSIYNYLAWYQQKPGKAPKLLIYGTSTLQSGVPSRFSGSGSGTDFTLTISSLQPEDFATYYCMQSYSSPTTFGQGTKVEIK

SEQ ID NO: 61 - protein, 118 aa, Artificial Sequence
  Synthetic
EVQLVESGGGLVQPGGSLRLSCAASGFTFKDYWLSWVRQAPGKGLEWVSIINYGGSETYYADSVKGRFTISRDNSKNTLYLQMNSLRAEDTAVYYCARTQTSYVMDYWGQGTLVTVSS

SEQ ID NO: 62 - protein, 107 aa, Artificial Sequence
  Synthetic
DIQMTQSPSSLSASVGDRVTITCRASQSVRSGLNWYQQKPGKAPKLLIYAASSLQSGVPRRFSGSGSGTDFTLTISSLQPEDFATYYCMQSHSWPLTFGQGTKVEIK

SEQ ID NO: 63 - protein, 120 aa, Artificial Sequence
  Synthetic
EVQLVESGGGLVQPGGSLRLSCAASGFTFSSGYMSWVRQAPGKGLEWVSTISGSGRETNYADSVKGRFTISRDNSKNTLYLQMNSLRAEDTAVYYCARDAWGGDSYFDPWGQGTLVTVSS

SEQ ID NO: 64 - protein, 107 aa, Artificial Sequence
  Synthetic
DIQMTQSPGSLSASVGDRVTITCRASQSIWSNLSWYQQKPGKAPKLLIYNASSLQSGVPSRFSGSGSGTDFTLTISSLQPEDFATYYCQQYHGTPITFGQGTKVEIK

SEQ ID NO: 65 - protein, 116 aa, Artificial Sequence
  Synthetic
EVQLVESGGGLVQPGGSLRLSCAASGFTFTDYQMSWVRQAPGKGLEWVSTIIWSGSETNYADSVKGRFTISRDNSKNTLYLQMNSLRAEDTAVYYCARNKTPFDYWGQGTLVTVSS

SEQ ID NO: 66 - protein, 107 aa, Artificial Sequence
  Synthetic
DIQMTQSPRSLSASVGDRVTITCRASQSIRTHLAWYQQKPGKAPKLLIYDNSNLQSGVPSRFSGSGSGTDFTLTISSLQPEDFATYYCQQYKGSPLTFGQGTKVEIK

SEQ ID NO: 67 - protein, 118 aa, Artificial Sequence
  Synthetic
EVQLVESGGGLVQPGGSLRLSCAASGFTFTSYAMSWVRQAPGKGLEWVSSISYSGGETDYADSVKGRFTISRDNSKNTLYLQMNSLRAEDTAVYYCARSKGGLYFDYWGQGTLVTVSS

SEQ ID NO: 68 - protein, 107 aa, Artificial Sequence
  Synthetic
DIQMTQSPGSLSASVGDRVTITCRASQSISSYLAWYQQKPGKAPKLLIYGASSLQSGVPSRFSGSGSGTDFTLTISSLQPEDFATYYCQQYGSTPETFGQGTKVEIK

SEQ ID NO: 69 - protein, 120 aa, Artificial Sequence
  Synthetic
EVQLVESGGGLVQPGGSLRLSCAASGFTFSGYWIHWVRQAPGKGLEWVSTISYSGDEAYYADSVKGRFTISRDNSKNTLYLQMNSLRAEDTAVYYCARSPSDGDYGFDYWGQGTLVTVSS

SEQ ID NO: 70 - protein, 107 aa, Artificial Sequence
  Synthetic
DIQMTQSPSSLSASVGDRVNITCRASQSINNYLSWYQQKPGKAPKLLIYDGRILQSGVPSRFSGSGSGTDFTLTISSLQPEDFATYYCMQYLAYPWTFGQGTKVEIK

SEQ ID NO: 71 - protein, 120 aa, Artificial Sequence
  Synthetic
EVQLVESGGGLVQPGGSLRLSCAASGFTFGTYDMHWVRQAPGKGLEWVSMISPSGGDTYYADSVKGRFTISRDNSKNTLYLQMNSLRAEDTAVYYCARDSSGDWYAMAYWGQGTLVTVSS

SEQ ID NO: 72 - protein, 107 aa, Artificial Sequence
  Synthetic
DIQMTQSPSSLSASVGDRVTITCRASQSIRRYLAWYQQKPGKAPKLLIYGASNLQSGVPSRFSGSGSGTDFTLTISSLQPEDFATYYCQHSSDTPLTFGQGTKVEIK

SEQ ID NO: 73 - protein, 120 aa, Artificial Sequence
  Synthetic
EVQLVESGGGLVQPGGSLRLSCAASGFTFSDSAMGWVRQAPGKGLEWVSIISYYGSETYYADSVKGRFTISRDNSKNTLYLQMNSLRAEDTAVYYCARNPDGDLSALDYWGQGTLVTVSS

SEQ ID NO: 74 - protein, 107 aa, Artificial Sequence
  Synthetic
DIQMTQSPSSLSASVGDRVTITCRASQPISSYLNWYQQKPGKAPKLLIYGASSLQSGVPSRFSGSGSGTDFTLTISSLQPEDFATYYCQQRLRSPFTFGQGTKVEIK

SEQ ID NO: 75 - protein, 123 aa, Artificial Sequence
  Synthetic
EVQLVESGGGLVQPGGSLRLSCAASGFTFTSYAMSWVRQAPGKGLEWVSSINSGGSETNYADSVKGRFTISRDNSKNTLYLQMNSLRAEDTAVYYCARGEYYTDVWPSGFDIWGQGTLVTVSS

SEQ ID NO: 76 - protein, 107 aa, Artificial Sequence
  Synthetic
DIQMTQSPSSLSASVGDRVTITCRASQSISNPLNWYQQKPGKAPKLLIYDASTLQSGVPSRFSGSGSGTDFTLTISSLQPEDFATYYCQQYGSSPSTFGQGTKVEIK

SEQ ID NO: 77 - protein, 119 aa, Artificial Sequence
  Synthetic
EVQLVESGGGLVQPGGSLRLSCAASGFTFSNYEMGWVRQAPGKGLEWVSIISWSGSETIYADSVKGRFTISRDNSKNTLYLQMNSLRAEDTAVYYCARNGRGDYAFDFWGQGTLVTVSS

SEQ ID NO: 78 - protein, 107 aa, Artificial Sequence
  Synthetic
DIQMTQSPSSLSASVGDRVTITCRASQSVSNNLAWYQQKPGKAPKLLIYRTTSLQSGVPSRFSGSGSGTDFTLTISSLQPEDFATYYCMQYGSLPSTFGQGTKVEIK

SEQ ID NO: 79 - protein, 120 aa, Artificial Sequence
  Synthetic
EVQLVESGGGLVQPGGSLRLSCAASGFTFSDYGMSWVRQAPGKGLEWVSIISPGGRETYYADSVKGRFTISRDNSKNTLYLQMNSLRAEDTAVYYCARSPDGGYYEFDVWGQGTLVTVSS

SEQ ID NO: 80 - protein, 107 aa, Artificial Sequence
  Synthetic
DIQMTQSPSSLSASVGDRVTITCRASQSISNYLAWYQQKPGKAPKLLIYDASTLQSGVPSRFSGSGSGTDFTLTISSLQPEDFATYYCHQRNSWPPTFGQGTKVEIK

SEQ ID NO: 81 - protein, 117 aa, Artificial Sequence
  Synthetic
EVQLVESGGGLVQPGGSLRLSCAASGFTFSSYHMHWVRQAPGKGLEWVSAIDSSGRETFYADSVKGRFTISRDNSKNTLYLQMNSLRAEDTAVYYCARGYGDYFDYWGQGTLVTVSS

SEQ ID NO: 82 - protein, 117 aa, Artificial Sequence; identical to SEQ ID NO: 81
  Synthetic

SEQ ID NO: 83 - protein, 119 aa, Artificial Sequence
  Synthetic
EVQLVESGGGLVQPGGSLRLSCAASGFTFDNYVMGWVRQAPGKGLEWVSMINYGGSETIYADSVKGRFTISRDNSKNTLYLQMNSLRAEDTAVYYCARSACDYCDFDYWGQGTLVTVSS

SEQ ID NO: 84 - protein, 107 aa, Artificial Sequence
  Synthetic
DIQMTQSPSSLSASVGDRVTITCRASQVVGSYLNWYQQKPGKAPKLLIYGASTLQSGVPSRFSGSGSGTDFTLTISSLQPEDFATYYCQQYYNYPGTFGQGTKVEIK

SEQ ID NO: 85 - protein, 120 aa, Artificial Sequence
  Synthetic
EVQLVESGGGLVQPGGSLRLSCAASGFTFTTYYMSWVRQAPGKGLEWVSTIIPSGSETNYADSVKGRFTISRDNSKNTLYLQMNSLRAEDTAVYYCARVPAASEGPMDYWGQGTLVTVSS

SEQ ID NO: 86 - protein, 107 aa, Artificial Sequence
  Synthetic
DIQMTQSPSSLSASVGDRVTITCRASQSISRNLAWYQQKPGKAPKLLIYDASSLQSGVPSRFSGSGSGTDFTLTISSLQPEDFATYYCQQYYHSPPTFGQGTKVEIK

SEQ ID NO: 87 - protein, 119 aa, Artificial Sequence
  Synthetic
EVQLVESGGGLVQPGGSLRLSCAASGFTFDSYAMHWVRQAPGKGLEWVSSIDGSGRETDYADSVKGRFTISRDNSKNTLYLQMNSLRAEDTAVYYCARDGSEGYAFDPWGQGTLVTVSS

SEQ ID NO: 88 - protein, 107 aa, Artificial Sequence
  Synthetic
DIQMTQSPSSLSASVGDRVTITCRASQIIRHKLNWYQQKPGKAPKLLIYDASTLQSGVPSRFSGSGSGTDFTLTISSLQPEDFATYYCQQYNSWPITFGQGAKVEIK

SEQ ID NO: 89 - protein, 119 aa, Artificial Sequence
  Synthetic
EVQLVESGGGLVQPGGSLRLSCAASGFTFTSYIMSWVRQAPGKGLEWVSIISYSGGETYYADSVKGRFTISRDNSKNTLYLQMNSLRAEDTAVYYCARNGINDDSFDYWGQGTLVTVSS

SEQ ID NO: 90 - protein, 107 aa, Artificial Sequence
  Synthetic
DIQMTQSPSSLSASVGDRVTITCRASQSISNYLNWYQQKPGKAPKLLIYGASSLQSGVPSRFSGSGSGTDFTLTISSLQPEDFATYYCQQRLHWPGTFGQGTKVEIK

SEQ ID NO: 91 - protein, 117 aa, Artificial Sequence
  Synthetic
EVQLVESGGGLVQPGGSLRLSCAASGFTFTNHWMSWVRQAPGKGLEWVSTIEGSGSETIYADSVKGRFTISRDNSKNTLYLQMNSLRAEDTAVYYCARSSRTLFDYWGQGTLVTVSS

SEQ ID NO: 92 - protein, 107 aa, Artificial Sequence
  Synthetic
DIQMTQSPSSLSASVGDRVTITCRASQGVYSYLAWYQQKPGKAPKLLIYDASSLQSGVPSRFSGSGSGTDFTLTISSLQPEDFATYYCQQYYHYPPTFGQGTKVEIK

SEQ ID NO: 93 - protein, 122 aa, Artificial Sequence
  Synthetic
EVQLVESGGGLVQPGGSLRLSCAASGFTFSNYYMDWVRQAPGKGLEWVSSINPWGGNKYYADSVKGRFTISRDNSKNTLYLQMNSLRAEDTAVYYCARTITSKYEDYAMDYWGQGTLVTVSS

SEQ ID NO: 94 - protein, 107 aa, Artificial Sequence
  Synthetic
DIQMTQSPSSLSASVGDRVTITCRASQSISSYLAWYQQKPGKAPKLLIYLTSNLQSGVPSRFSGSGSGTDFTLTISSLQPEDFATYYCQQTAQNPFTFGQGTKVEIK

SEQ ID NO: 95 - protein, 120 aa, Artificial Sequence
  Synthetic
EVQLVESGGGLVQPGGSLRLSCAASGFTFSDYWVAWVRQAPGKGLEWVSTISYSGSETEYADSVKGRFTISRDNSKNTLYLQMNSLRAEDTAVYYCARYGGSDYYGFDPWGQGTLVTVSS

SEQ ID NO: 96 - protein, 106 aa, Artificial Sequence
  Synthetic
DIQMTQSPSSLSASVGDRVTITCRASQSISNNLAWYQQKPGKAPKLLIYATTTLQSGVPSRFSGSGSGTDFTLTISSLQPEDFATYYCMQSNTPWTFGQGTKVEIK

SEQ ID NO: 97 - protein, 120 aa, Artificial Sequence
  Synthetic
EVQLVESGGGLVQPGGSLRLSCAASGFTFSSYAISWVRQAPGKGLEWVSAISSGGSETDYADSVKGRFTISRDNSKNTLYLQMNSLRAEDTAVYYCARGESGYYMAEDVWGQGTLVTVSS

SEQ ID NO: 98 - protein, 107 aa, Artificial Sequence
  Synthetic
DIQMTQSPSSLSASVGDRVTITCRASQSVSSFLAWYQQKPGKAPKLLIYAASKLQSGVPSRFSGSGSGTDFTLTISSLQPEDFATYYCQQYSVTPITFGQGTKVEIK

SEQ ID NO: 99 - protein, 123 aa, Artificial Sequence
  Synthetic
EVQLVESGGGLVQPGGSLRLSCAASGFTFSSYAMSWVRQAPGKGLEWVSAISSYGGETDYADSVKGRFTISRDNSKNTLYLQMNSLRAEDTAVYYCARGDAYSSFVDNPFDIWGQGTLVTVSS

SEQ ID NO: 100 - protein, 107 aa, Artificial Sequence
  Synthetic
DIQMTQSPSSLSASVGDRVTITCRASQSISNYLAWYQQKPGKAPKLLIYDASTLQSGVPSRFSGSGSGTDFTLTISSLQPEDFATYYCMQYESTPWTFGQGTKVEIK

SEQ ID NO: 101 - protein, 119 aa, Artificial Sequence
  Synthetic
EVQLVESGGGLVQPGGSLRLSCAASGFTFTSYAMSWVRQAPGKGLEWVSAISPSGSETEYADSVKGRFTISRDNSKNTLYLQMNSLRAEDTAVYYCARGFYNDYIFDLWGQGTLVTVSS

SEQ ID NO: 102 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQYLSTPYTFGQGTKVEIK

SEQ ID NO: 103 - protein, 123 aa, Artificial Sequence
  Synthetic
EVQLVESGGGLVQPGGSLRLSCAASGFTFRNNAMHWVRQAPGKGLEWVSVINSGGSETYYADSVKGRFTISRDNSKNTLYLQMNSLRAEDTAVYYCARDEPSDEYGMYGFDYWGQGTLVTVSS

SEQ ID NO: 104 - protein, 107 aa, Artificial Sequence
  Synthetic
DIQMTQSPSSLSASVGDRVTITCRASQSISSYLNWYQQKPGKAPKLLIYKASNLQSGVPSRFSGSGSGTDFTLTISSLQPEDFATYYCVQYSRSPTTFGQGTKVEIK

SEQ ID NO: 105 - protein, 120 aa, Artificial Sequence
  Synthetic
EVQLVESGGGLVQPGGSLRLSCAASGFTFTSATMSWVRQAPGKGLEWVSIISPGGSETYYADSVKGRFTISRDNSKNTLYLQMNSLRAEDTAVYYCARGGDYPSYYMDPWGQGTLVTVSS

SEQ ID NO: 106 - protein, 107 aa, Artificial Sequence
  Synthetic
DIQMTQSPSSLSASVGDRVTITCRASQSISNYLAWYQQKPGKAPKLLIYGTSSLQSGVPSRFSGSGSGTDFTLTISSLQPEDFATYYCQQGHQWPWTFGQGTKVEIK

SEQ ID NO: 107 - protein, 119 aa, Artificial Sequence
  Synthetic
EVQLVESGGGLVQPGGSLRLSCAASGFTFTSYYMSWVRQAPGKGLEWVSTIISDGSETGYADSVKGRFTISRDNSKNTLYLQMNSLRAEDTAVYYCARTNRYGFQFDYWGQGTLVTVSS

SEQ ID NO: 108 - protein, 107 aa, Artificial Sequence
  Synthetic
DIQMTQSPSSLSASVGDRVTITCRASQGARNGLHWYQQKPGKAPKLLIYDASTLQSGVPSRFSGSGSGTDFTLTISSLQPEDFATYYCQQRYSYPPTFGQGTKVEIK

SEQ ID NO: 109 - protein, 120 aa, Artificial Sequence
  Synthetic
QVQLQQSGPELVKPGASVKISCKASGYSFTDYNMNWVKQSNGKSLEWIGEINPGYGTSRYNQKFKDKATLTVDQSSTTAYMQLNSLTSEDSAVYYCARADVDYGDVMDYWGQGTTVTVSS

SEQ ID NO: 110 - protein, 106 aa, Artificial Sequence
  Synthetic
DIELTQSPAIMSASPGEKVTMTCSASSSVSYMHWYQQKSGTSPKRWIYDTSKLASGVPTRFSGSGSGTSYSLTISSMEAEDAATYYCQQWSSNPPTFGAGTKLELK

SEQ ID NO: 111 - protein, 120 aa, Artificial Sequence
  Synthetic
QVKLQESGPELVKPGASVKISCKASGYSFTDYNMNWVKQSNGKSLEWIGEINPGYGTSRYNQKFKDKATLTVDQSSSTAYMQLNSLTSEDSAVYYCARADVDYGDTMDYWGQGTTVTVSS

SEQ ID NO: 112 - protein, 106 aa, Artificial Sequence
  Synthetic
DIELTQSPAIMSASPGEKVTMTCSASSSVSDMHWYQQKSGNSPKRWIYDTSKLASGVPVRFSGSGSGTSYSLTISSMEAEDAATYYCQQWSSNPPTFGAGTKLELK

SEQ ID NO: 113 - protein, 120 aa, Artificial Sequence
  Synthetic
QVKLQESGPELVKPGASVKISCKASGYSFTDYNMNWVKQSNGKSLEWIGVINPNYGTTSYNQKFKGKATLTVDQSSSTAYMQLNSLTSEDSAVYYCARTFDYDDDAFAYWGQGTTVTVSS

SEQ ID NO: 114 - protein, 106 aa, Artificial Sequence
  Synthetic
DIELTQSPAIMSASPGEKVTITCSASSSVSDMHWYQQKSGTSPKRWIYDTSKLASGVPARFSGSGSGTSYSLTISNMEAEDAATYYCQQWSSNPPTFGAGTKLELK

SEQ ID NO: 115 - protein, 120 aa, Artificial Sequence
  Synthetic
QVKLQQSGPELVKPGASVKISCKASGYSFTDYNMNWVKQSNGKSLEWIGVINPNYGTTSYNQKFKGKATLTVDQSSSTAYMQLNSLTSEDSAVYYCARTYDYDGDAFAYWGQGTTVTVSS

SEQ ID NO: 116 - protein, 106 aa, Artificial Sequence
  Synthetic
DIELTQSPAIMSASPGEKVTMTCSASSSVSYMHWYQQKSGTSPKRWIYDTSKLASGVPARFSGSGSGTSYSLTISSMEAEDAATYYCQQWSSNPPTFGAGTKLELK

SEQ ID NO: 117 - protein, 120 aa, Artificial Sequence
  Synthetic
QVKLQQSGPELVTPGASVKISCKPSGYSFTDYNMNWVKQSNGKSLEWIGEINPNYGTTRHNQKFKGKASLTVDQSSSTAYMQLISLTSEDSAVYYCARTYDYDEDAFAYWGQGTTVTVSS

SEQ ID NO: 118 - protein, 106 aa, Artificial Sequence
  Synthetic
DIELTQSPAIMSASPGEKVTMTCSASSSVSYMHWYQQKSDTSPKRWIYDTSKLASGVPARFSGSGSGTSYSLTISSMEAEDAATYYCQQWSSNPPTFGAGTKLELK

SEQ ID NO: 119 - protein, 120 aa, Artificial Sequence
  Synthetic
QVKLQQSGPELVKPGASVKISCKASGYSFTDYNMNWVKQSNGKSLEWIGIINPNYGTTSYNQKFKGKATLTVDQSSSTAYMQLNSLTSEDSAVYYCARGYDYDESGFAYWGQGTTVTVSS

SEQ ID NO: 120 - protein, 120 aa, Artificial Sequence; identical to SEQ ID NO: 119
  Synthetic

SEQ ID NO: 121 - protein, 120 aa, Artificial Sequence
  Synthetic
QVQLQQSGPELVKPGASVKISCKTSGYSFTDYNMNWVKQSNGKSLEWIGRINPNFGTTTYNQKFKGKATLTVDQSSSTAYMQLNSLTSEDSAVYYCARGYDYDESGFAYWGQGTTVTVSS

SEQ ID NO: 122 - protein, 106 aa, Artificial Sequence
  Synthetic
DIELTQSPAIMSASPGEKVTMTCSASSSVSYMYWYQQKPGSSPRLLIYDTSNLASGVPVRFSGSGSGTSYSLTISRMEAEDAATYYCQQWSSYPPTFGGGTKLEIK

SEQ ID NO: 123 - protein, 120 aa, Artificial Sequence
  Synthetic
QVKLQESGAEVVKPGASVKISCKASGYSFTDYNMNWVKQSNGKSLEWIGVINPNYGTTSYNQKFKGKATLTVDQSSSTAYMQLNSLTSEDSAVYYCARTYDYDGDAFAYWGQGTTVTVSS

SEQ ID NO: 124 - protein, 108 aa, Artificial Sequence
  Synthetic
DIELTQSPALMSASPGEKVTMTCRASSSVSSNNLHWYQQKSGASPKLWIYSTSNLATGAPARFSGSGSGTSYSLTISSMEAEDAATYYCQQWNSNPPTFGGGTKLEIK

SEQ ID NO: 125 - protein, 122 aa, Artificial Sequence
  Synthetic
QVKLQQSGPELVKPGASVKISCKASGYSFTDYNMNWVKQSNGKSLEWIGEINPNYGTTRYNQKFKGKATLTVDQSSSTAYMQLNSLTSEDSAVYYCARPSIYYDYDDAMDYWGQGTTVTVSS

SEQ ID NO: 126 - protein, 108 aa, Artificial Sequence
  Synthetic
DIELTQSPAIMSASPGEKVTMTCSASSSVNYMYWYQQKPGSSPRLLIYDTSNLASGVPVRFSGSGSGTSYSLTISRMEAEDAATYYCQQWITYPPTLTFGAGTKLEIK

SEQ ID NO: 127 - protein, 118 aa, Artificial Sequence
  Synthetic
QVQLQQSGTVLARPGASVKMSCKASGYTFTSYWMHWVKQRPGQGLEWIGAIYPGNSDTSYNQKFKGKAKLTAVTSASTAYMELSSLTNEDSAVYYCTRGGGSYEFAYWGQGTTVTVSS

SEQ ID NO: 128 - protein, 106 aa, Artificial Sequence
  Synthetic
DIELTQSPAIMSASPGEKVTMTCSVSSSVSYMYWYQQKPGSSPRLLIYDTSNLASGVPARFSGSGSGTSYSLTISSMEAEDAATYYCQQWSSNPFTFGSGTKLEIK

SEQ ID NO: 129 - protein, 117 aa, Artificial Sequence
  Synthetic
QVQLQESGPGLVKPSETLSLTCTVSGFSLTSNSISWVRQPPGKGLEWMGAIWSGGGTDYNPSLKSRLTISRDTSKSQVFLKMSSLTAADTAIYFCTRGGYPYYFDYWGQGVLVTVSS

SEQ ID NO: 130 - protein, 113 aa, Artificial Sequence
  Synthetic
DIVMTQSPDSLAVSLGERVTINCKSSQSLLYSENQENYLAWYQQKPGQSPKLLIYWASTRQSGVPDRFSGSGSGTDFTLTISSVQAEDLAIYYCQQYYDTPLTFGGGTKLEIK

SEQ ID NO: 131 - protein, 118 aa, Artificial Sequence
  Synthetic
QVQLVQSGAEVKKPGASVKVSCKASGYTFTNYWINWVRQAPGQRLEWMGNIYPSDTYINHNQKFKDRVTITRDTSASTAYMELSSLRSEDTAVYYCARSAYANYFDYWGQGTLVTVSS

SEQ ID NO: 132 - protein, 108 aa, Artificial Sequence
  Synthetic
EIVMTQSPATLSVSPGERATLSCRASQNIGTSIHWYQQKPGQAPRLLIYYASESISGIPARFSGSGSGTEFTLTISSLQSEDFAVYYCQQSDSWPTLTFGQGTKLEIK

SEQ ID NO: 133 - protein, 120 aa, Artificial Sequence
  Synthetic
QVQLVQSGAEVKKPGASVKVSCKASGYSFTDYNMNWVRQAPGQGLEWMGEINPGYGTSRYNQKFKDRVTMTRDTSISTAYMELSRLRSDDTAVYYCARADVDYGDVMDYWGQGTLVTVSS

SEQ ID NO: 134 - protein, 106 aa, Artificial Sequence
  Synthetic
EIVLTQSPATLSLSPGERATLSCSASSSVSYMHWYQQKPGQAPRLLIYDTSKLASGIPARFSGSGSGTDFTLTISSLEPEDFAVYYCQQWSSNPPTFGQGTKLEIK

SEQ ID NO: 135 - protein, 120 aa, Artificial Sequence
  Synthetic
QVQLVQSGAEVKKPGASVKVSCKASGYSFTDYNMNWVRQAPGQGLEWMGEINPGYGTSRYNQKFKDRVTLTVDQSITTAYMELSRLRSDDTAVYYCARADVDYGDVMDYWGQGTLVTVSS

SEQ ID NO: 136 - protein, 106 aa, Artificial Sequence; identical to SEQ ID NO: 134
  Synthetic

SEQ ID NO: 137 - protein, 120 aa, Artificial Sequence
  Synthetic
QVQLVQSGAEVKKPGASVKVSCKASGYSFTDYNMNWVRQAPGQGLEWIGEINPGYGTSRYNQKFKDKATLTVDQSITTAYMELSRLRSDDTAVYYCARADVDYGDVMDYWGQGTLVTVSS

SEQ ID NO: 138 - protein, 106 aa, Artificial Sequence; identical to SEQ ID NO: 134
  Synthetic

SEQ ID NO: 139 - protein, 120 aa, Artificial Sequence
  Synthetic
QVQLVQSGAEVKKPGASVKISCKASGYSFTDYNMNWVKQAPGQGLEWIGEINPGYGTSRYNQKFKDKATLTVDQSITTAYMELSRLRSDDTAVYYCARADVDYGDVMDYWGQGTLVTVSS

SEQ ID NO: 140 - protein, 106 aa, Artificial Sequence; identical to SEQ ID NO: 134
  Synthetic

SEQ ID NO: 141 - protein, 120 aa, Artificial Sequence
  Synthetic
QVQLVQSGPEVVKPGASVKISCKASGYSFTDYNMNWVKQANGQSLEWIGEINPGYGTSRYNQKFKDKATLTVDQSITTAYMELNRLRSDDTAVYYCARADVDYGDVMDYWGQGTLVTVSS

SEQ ID NO: 142 - protein, 106 aa, Artificial Sequence; identical to SEQ ID NO: 134
  Synthetic

SEQ ID NO: 143 - protein, 120 aa, Artificial Sequence; identical to SEQ ID NO: 133
  Synthetic

SEQ ID NO: 144 - protein, 106 aa, Artificial Sequence
  Synthetic
EIVLTQSPATLSLSPGERATLSCSASSSVSYMHWYQQKPGQAPRRLIYDTSKLASGIPARFSGSGSGTDFTLTISSLEPEDFAVYYCQQWSSNPPTFGQGTKLEIK

SEQ ID NO: 145 - protein, 120 aa, Artificial Sequence; identical to SEQ ID NO: 133
  Synthetic

SEQ ID NO: 146 - protein, 106 aa, Artificial Sequence
  Synthetic
EIVLTQSPATLSLSPGERATLSCSASSSVSYMHWYQQKPGQAPRRWIYDTSKLASGIPARFSGSGSGTDYTLTISSLEPEDFAVYYCQQWSSNPPTFGQGTKLEIK

SEQ ID NO: 147 - protein, 120 aa, Artificial Sequence; identical to SEQ ID NO: 133
  Synthetic

SEQ ID NO: 148 - protein, 106 aa, Artificial Sequence
  Synthetic
EIELTQSPATLSASPGERVTMSCSASSSVSYMHWYQQKPGQAPKRWIYDTSKLASGVPARFSGSGSGTDYTLTISSMEPEDFATYYCQQWSSNPPTFGQGTKLEIK

SEQ ID NO: 149 - protein, 120 aa, Artificial Sequence; identical to SEQ ID NO: 133
  Synthetic

SEQ ID NO: 150 - protein, 106 aa, Artificial Sequence
  Synthetic
EIELTQSPATMSASPGERVTMSCSASSSVSYMHWYQQKSGQSPKRWIYDTSKLASGVPARFSGSGSGTDYTLTISSMEPEDFATYYCQQWSSNPPTFGQGTKLEIK

SEQ ID NO: 151 - protein, 120 aa, Artificial Sequence; identical to SEQ ID NO: 135
  Synthetic

SEQ ID NO: 152 - protein, 106 aa, Artificial Sequence; identical to SEQ ID NO: 144
  Synthetic

SEQ ID NO: 153 - protein, 120 aa, Artificial Sequence; identical to SEQ ID NO: 135
  Synthetic

SEQ ID NO: 154 - protein, 106 aa, Artificial Sequence; identical to SEQ ID NO: 146
  Synthetic

SEQ ID NO: 155 - protein, 120 aa, Artificial Sequence; identical to SEQ ID NO: 137
  Synthetic

SEQ ID NO: 156 - protein, 106 aa, Artificial Sequence; identical to SEQ ID NO: 144
  Synthetic

SEQ ID NO: 157 - protein, 120 aa, Artificial Sequence; identical to SEQ ID NO: 137
  Synthetic

SEQ ID NO: 158 - protein, 106 aa, Artificial Sequence; identical to SEQ ID NO: 146
  Synthetic

SEQ ID NO: 159 - protein, 10 aa, Artificial Sequence
  Synthetic
GFTFSSYWMS

SEQ ID NO: 160 - protein, 17 aa, Artificial Sequence
  Synthetic
IINYSGREADYADSVKG

SEQ ID NO: 161 - protein, 14 aa, Artificial Sequence
  Synthetic
DGSYVGQAHEAFDI

SEQ ID NO: 162 - protein, 11 aa, Artificial Sequence
  Synthetic
RASQSISKYLA

SEQ ID NO: 163 - protein, 7 aa, Artificial Sequence
  Synthetic
GTSNLQS

SEQ ID NO: 164 - protein, 9 aa, Artificial Sequence
  Synthetic
QQSRSWPLT

SEQ ID NO: 165 - protein, 10 aa, Artificial Sequence
  Synthetic
GFTFSSAWMS

SEQ ID NO: 166 - protein, 17 aa, Artificial Sequence
  Synthetic
TIYWSGSEINYADSVKG

SEQ ID NO: 167 - protein, 11 aa, Artificial Sequence
  Synthetic
SFASGESAMDV

SEQ ID NO: 168 - protein, 11 aa, Artificial Sequence
  Synthetic
RASQSISNYLA

SEQ ID NO: 169 - protein, 7 aa, Artificial Sequence
  Synthetic
DASNLQS

SEQ ID NO: 170 - protein, 7 aa, Artificial Sequence
  Synthetic
DASNLQS

SEQ ID NO: 171 - protein, 11 aa, Artificial Sequence
  Synthetic
GFTFSTSWMHW

SEQ ID NO: 172 - protein, 11 aa, Artificial Sequence
  Synthetic
GFTFSTSWMHW

SEQ ID NO: 173 - protein, 7 aa, Artificial Sequence
  Synthetic
GDAAFDY

SEQ ID NO: 174 - protein, 11 aa, Artificial Sequence
  Synthetic
RASQLIRKKLA

SEQ ID NO: 175 - protein, 7 aa, Artificial Sequence
  Synthetic
AASTLQS

SEQ ID NO: 176 - protein, 9 aa, Artificial Sequence
  Synthetic
MQSGSPQYT

SEQ ID NO: 177 - protein, 10 aa, Artificial Sequence
  Synthetic
GFTFTNYWMG

SEQ ID NO: 178 - protein, 17 aa, Artificial Sequence
  Synthetic
IIISDGGEIIYADSVKG

SEQ ID NO: 179 - protein, 11 aa, Artificial Sequence
  Synthetic
IHAGTGSSADY

SEQ ID NO: 180 - protein, 11 aa, Artificial Sequence
  Synthetic
RASQSIYNYLN

SEQ ID NO: 181 - protein, 7 aa, Artificial Sequence
  Synthetic
DASTLQS

SEQ ID NO: 182 - protein, 9 aa, Artificial Sequence
  Synthetic
QQYYSYPWT

SEQ ID NO: 183 - protein, 10 aa, Artificial Sequence
  Synthetic
GFTFRTFGMN

SEQ ID NO: 184 - protein, 18 aa, Artificial Sequence
  Synthetic
GIVPSGSEIDYADSVKGR

SEQ ID NO: 185 - protein, 11 aa, Artificial Sequence
  Synthetic
DHSHYTGPFDV

SEQ ID NO: 186 - protein, 11 aa, Artificial Sequence
  Synthetic
RASQSIYSYLN

SEQ ID NO: 187 - protein, 7 aa, Artificial Sequence
  Synthetic
GASILQY

SEQ ID NO: 188 - protein, 7 aa, Artificial Sequence
  Synthetic
GASILQY

SEQ ID NO: 189 - protein, 10 aa, Artificial Sequence
  Synthetic
GFTFTSYWMT

SEQ ID NO: 190 - protein, 17 aa, Artificial Sequence
  Synthetic
TIVGGGGEADYADSVKG

SEQ ID NO: 191 - protein, 12 aa, Artificial Sequence
  Synthetic
DYVADNHGAMDY

SEQ ID NO: 192 - protein, 11 aa, Artificial Sequence
  Synthetic
RASQGLSSYLN

SEQ ID NO: 193 - protein, 7 aa, Artificial Sequence
  Synthetic
GMSTLQS

SEQ ID NO: 194 - protein, 9 aa, Artificial Sequence
  Synthetic
MQYYHWPYT

SEQ ID NO: 195 - protein, 10 aa, Artificial Sequence
  Synthetic
GFTFSDYIMH

SEQ ID NO: 196 - protein, 17 aa, Artificial Sequence
  Synthetic
AISPSGSEIYYADSVKG

SEQ ID NO: 197 - protein, 15 aa, Artificial Sequence
  Synthetic
NAWDNNWVREYGMDY

SEQ ID NO: 198 - protein, 11 aa, Artificial Sequence
  Synthetic
RASQSGNNNLA

SEQ ID NO: 199 - protein, 7 aa, Artificial Sequence
  Synthetic
GASTLQS

SEQ ID NO: 200 - protein, 9 aa, Artificial Sequence
  Synthetic
MQSNSYPLT

SEQ ID NO: 201 - protein, 9 aa, Artificial Sequence
  Synthetic
MQSNSYPLT

SEQ ID NO: 202 - protein, 8 aa, Artificial Sequence
  Synthetic
IYADSVKG

SEQ ID NO: 203 - protein, 11 aa, Artificial Sequence
  Synthetic
SSYGEGYAFDY

SEQ ID NO: 204 - protein, 11 aa, Artificial Sequence
  Synthetic
RASQDVDSYLN

SEQ ID NO: 205 - protein, 7 aa, Artificial Sequence
  Synthetic
DAISLQS

SEQ ID NO: 206 - protein, 9 aa, Artificial Sequence
  Synthetic
QQYYSLPLT

SEQ ID NO: 207 - protein, 10 aa, Artificial Sequence
  Synthetic
GFTFSDYAIG

SEQ ID NO: 208 - protein, 17 aa, Artificial Sequence
  Synthetic
TIYWSGSNAYYADSVKG

SEQ ID NO: 209 - protein, 11 aa, Artificial Sequence
  Synthetic
LFTLGYHGFDV

SEQ ID NO: 210 - protein, 11 aa, Artificial Sequence
  Synthetic
RASQSINTYLN

SEQ ID NO: 211 - protein, 7 aa, Artificial Sequence
  Synthetic
DASNLQS

SEQ ID NO: 212 - protein, 9 aa, Artificial Sequence
  Synthetic
QQYDDLPFT

SEQ ID NO: 213 - protein, 11 aa, Artificial Sequence
  Synthetic
SGFTFSSNSMS

SEQ ID NO: 214 - protein, 17 aa, Artificial Sequence
  Synthetic
AINYSGREIYYADSVKG

SEQ ID NO: 215 - protein, 8 aa, Artificial Sequence
  Synthetic
TDYNTFDY

SEQ ID NO: 216 - protein, 11 aa, Artificial Sequence
  Synthetic
RASQSNSSHLN

SEQ ID NO: 217 - protein, 7 aa, Artificial Sequence
  Synthetic
GVSNLQS

SEQ ID NO: 218 - protein, 9 aa, Artificial Sequence
  Synthetic
QHYGSTPYT

SEQ ID NO: 219 - protein, 10 aa, Artificial Sequence
  Synthetic
GFTFSDYYMS

SEQ ID NO: 220 - protein, 17 aa, Artificial Sequence
  Synthetic
VISYGGGEAYYADSVKG

SEQ ID NO: 221 - protein, 11 aa, Artificial Sequence
  Synthetic
AMASEYGPWDY

SEQ ID NO: 222 - protein, 11 aa, Artificial Sequence
  Synthetic
RASQSISRHLT

SEQ ID NO: 223 - protein, 7 aa, Artificial Sequence
  Synthetic
GASTLQS

SEQ ID NO: 224 - protein, 9 aa, Artificial Sequence
  Synthetic
QQYHDWPLT

SEQ ID NO: 225 - protein, 10 aa, Artificial Sequence
  Synthetic
GFTFGDDYMH

SEQ ID NO: 226 - protein, 17 aa, Artificial Sequence
  Synthetic
AINYDGSWKYYADSVKG

SEQ ID NO: 227 - protein, 14 aa, Artificial Sequence
  Synthetic
LSSIDEPPYGPFDV

SEQ ID NO: 228 - protein, 11 aa, Artificial Sequence
  Synthetic
RASQSISTYLA

SEQ ID NO: 229 - protein, 7 aa, Artificial Sequence
  Synthetic
EASSLQS

SEQ ID NO: 230 - protein, 9 aa, Artificial Sequence
  Synthetic
QQYYNFPPT

SEQ ID NO: 231 - protein, 10 aa, Artificial Sequence
  Synthetic
GFTFNNYYMS

SEQ ID NO: 232 - protein, 17 aa, Artificial Sequence
  Synthetic
IIYYDGSEADYADSVKG

SEQ ID NO: 233 - protein, 12 aa, Artificial Sequence
  Synthetic
NKDIYSVYGMDY

SEQ ID NO: 234 - protein, 11 aa, Artificial Sequence
  Synthetic
RASQSISNYLA

SEQ ID NO: 235 - protein, 7 aa, Artificial Sequence
  Synthetic
ATSNLQS

SEQ ID NO: 236 - protein, 9 aa, Artificial Sequence
  Synthetic
QQANNTPPT

SEQ ID NO: 237 - protein, 10 aa, Artificial Sequence
  Synthetic
GFTFSDYWMS

SEQ ID NO: 238 - protein, 17 aa, Artificial Sequence
  Synthetic
SIVYGGSEIDYADSVKG

SEQ ID NO: 239 - protein, 11 aa, Artificial Sequence
  Synthetic
VPGYSGTPFDY

SEQ ID NO: 240 - protein, 11 aa, Artificial Sequence
  Synthetic
RASQSVSRYLA

SEQ ID NO: 241 - protein, 7 aa, Artificial Sequence
  Synthetic
DTSSLQS

SEQ ID NO: 242 - protein, 9 aa, Artificial Sequence
  Synthetic
QQSYSFPLT

SEQ ID NO: 243 - protein, 10 aa, Artificial Sequence
  Synthetic
GFTFKDSWMH

SEQ ID NO: 244 - protein, 17 aa, Artificial Sequence
  Synthetic
IISYSGGEAIYADSVKG

SEQ ID NO: 245 - protein, 11 aa, Artificial Sequence
  Synthetic
DSGGSAMGFDI

SEQ ID NO: 246 - protein, 11 aa, Artificial Sequence
  Synthetic
RASQSIHSYLN

SEQ ID NO: 247 - protein, 7 aa, Artificial Sequence
  Synthetic
GASSLQS

SEQ ID NO: 248 - protein, 9 aa, Artificial Sequence
  Synthetic
QQYYRFPYT

SEQ ID NO: 249 - protein, 10 aa, Artificial Sequence
  Synthetic
GFTFSDWYLH

SEQ ID NO: 250 - protein, 17 aa, Artificial Sequence
  Synthetic
VINGGGSEIIYADSVKG

SEQ ID NO: 251 - protein, 11 aa, Artificial Sequence
  Synthetic
GGDGDGSGFDD

SEQ ID NO: 252 - protein, 11 aa, Artificial Sequence
  Synthetic
RASQSIHSYLN

SEQ ID NO: 253 - protein, 7 aa, Artificial Sequence
  Synthetic
DASNLQS

SEQ ID NO: 254 - protein, 9 aa, Artificial Sequence
  Synthetic
QQSGNYPFT

SEQ ID NO: 255 - protein, 10 aa, Artificial Sequence
  Synthetic
GFTFSFTAMH

SEQ ID NO: 256 - protein, 17 aa, Artificial Sequence
  Synthetic
AIIYNGSEADYADSVKG

SEQ ID NO: 257 - protein, 16 aa, Artificial Sequence
  Synthetic
ANDYDHGCCDNYAMDY

SEQ ID NO: 258 - protein, 11 aa, Artificial Sequence
  Synthetic
RASQGIGSYLY

SEQ ID NO: 259 - protein, 7 aa, Artificial Sequence
  Synthetic
DASNLQS

SEQ ID NO: 260 - protein, 9 aa, Artificial Sequence
  Synthetic
MQHGGWPLT

SEQ ID NO: 261 - protein, 10 aa, Artificial Sequence
  Synthetic
GFTFSSYYMS

SEQ ID NO: 262 - protein, 17 aa, Artificial Sequence
  Synthetic
TINYDGSEKDYADSVKG

SEQ ID NO: 263 - protein, 11 aa, Artificial Sequence
  Synthetic
DRLGNYPWFDV

SEQ ID NO: 264 - protein, 11 aa, Artificial Sequence
  Synthetic
RASQSISNYLA

SEQ ID NO: 265 - protein, 7 aa, Artificial Sequence
  Synthetic
DANNLQS

SEQ ID NO: 266 - protein, 9 aa, Artificial Sequence
  Synthetic
QQSYSWPLT

SEQ ID NO: 267 - protein, 10 aa, Artificial Sequence
  Synthetic
GFTFTEWWMS

SEQ ID NO: 268 - protein, 17 aa, Artificial Sequence
  Synthetic
TISYGGSEAIYADSVKG

SEQ ID NO: 269 - protein, 10 aa, Artificial Sequence
  Synthetic
TSSDRLLFDY

SEQ ID NO: 270 - protein, 11 aa, Artificial Sequence
  Synthetic
RASQSIKSSLA

SEQ ID NO: 271 - protein, 7 aa, Artificial Sequence
  Synthetic
GASTLQS

SEQ ID NO: 272 - protein, 9 aa, Artificial Sequence
  Synthetic
MQTNRHPWT

SEQ ID NO: 273 - protein, 10 aa, Artificial Sequence
  Synthetic
GFTFSNYNLH

SEQ ID NO: 274 - protein, 17 aa, Artificial Sequence
  Synthetic
TISYSGSEIIYADSVKG

SEQ ID NO: 275 - protein, 11 aa, Artificial Sequence
  Synthetic
EDEYTYYYFDP

SEQ ID NO: 276 - protein, 11 aa, Artificial Sequence
  Synthetic
RASQSVSSYLA

SEQ ID NO: 277 - protein, 7 aa, Artificial Sequence
  Synthetic
DASKLQS

SEQ ID NO: 278 - protein, 9 aa, Artificial Sequence
  Synthetic
KQSYSSPPT

SEQ ID NO: 279 - protein, 10 aa, Artificial Sequence
  Synthetic
GFTFSDYWMH

SEQ ID NO: 280 - protein, 17 aa, Artificial Sequence
  Synthetic
TIIGGGSEAGYADSVKG

SEQ ID NO: 281 - protein, 11 aa, Artificial Sequence
  Synthetic
DRSYGYLGFDI

SEQ ID NO: 282 - protein, 11 aa, Artificial Sequence
  Synthetic
RASQSIRNSLH

SEQ ID NO: 283 - protein, 7 aa, Artificial Sequence
  Synthetic
SAGKLQS

SEQ ID NO: 284 - protein, 9 aa, Artificial Sequence
  Synthetic
QQSNSFPLT

SEQ ID NO: 285 - protein, 10 aa, Artificial Sequence
  Synthetic
GFTFSTNWMH

SEQ ID NO: 286 - protein, 17 aa, Artificial Sequence
  Synthetic
MISYSGGEAIYADSVKG

SEQ ID NO: 287 - protein, 9 aa, Artificial Sequence
  Synthetic
NWLPYAMDY

SEQ ID NO: 288 - protein, 11 aa, Artificial Sequence
  Synthetic
RASQSIRSYLS

SEQ ID NO: 289 - protein, 7 aa, Artificial Sequence
  Synthetic
SASTLQS

SEQ ID NO: 290 - protein, 9 aa, Artificial Sequence
  Synthetic
QQSYSTPLT

SEQ ID NO: 291 - protein, 10 aa, Artificial Sequence
  Synthetic
GFTFSSYDVG

SEQ ID NO: 292 - protein, 17 aa, Artificial Sequence
  Synthetic
GIVPSGGNIYYADSVKG

SEQ ID NO: 293 - protein, 11 aa, Artificial Sequence
  Synthetic
HRSYAYYAFDY

SEQ ID NO: 294 - protein, 11 aa, Artificial Sequence
  Synthetic
RASQNVRNYLG

SEQ ID NO: 295 - protein, 7 aa, Artificial Sequence
  Synthetic
DASSLQS

SEQ ID NO: 296 - protein, 9 aa, Artificial Sequence
  Synthetic
QQYGDWPLT

SEQ ID NO: 297 - protein, 10 aa, Artificial Sequence
  Synthetic
GFTFTDAYMS

SEQ ID NO: 298 - protein, 17 aa, Artificial Sequence
  Synthetic
TISSYGSSTYYADSVKG

SEQ ID NO: 299 - protein, 11 aa, Artificial Sequence
  Synthetic
PRYIESPVYDY

SEQ ID NO: 300 - protein, 11 aa, Artificial Sequence
  Synthetic
RASQSIAKYLA

SEQ ID NO: 301 - protein, 7 aa, Artificial Sequence
  Synthetic
ETSNLQS

SEQ ID NO: 302 - protein, 9 aa, Artificial Sequence
  Synthetic
QQSSSSPET

SEQ ID NO: 303 - protein, 10 aa, Artificial Sequence
  Synthetic
GFTFTNYWMH

SEQ ID NO: 304 - protein, 17 aa, Artificial Sequence
  Synthetic
TIIYDGGETGYADSVKG

SEQ ID NO: 305 - protein, 9 aa, Artificial Sequence
  Synthetic
NSRKSGMDY

SEQ ID NO: 306 - protein, 11 aa, Artificial Sequence
  Synthetic
RASQSIYKYLN

SEQ ID NO: 307 - protein, 7 aa, Artificial Sequence
  Synthetic
DASTLQS

SEQ ID NO: 308 - protein, 9 aa, Artificial Sequence
  Synthetic
MQYYSDPLT

SEQ ID NO: 309 - protein, 10 aa, Artificial Sequence
  Synthetic
GFTFSGYAWS

SEQ ID NO: 310 - protein, 17 aa, Artificial Sequence
  Synthetic
MISPAGGSTYYADSVKG

SEQ ID NO: 311 - protein, 14 aa, Artificial Sequence
  Synthetic
DRNAGGDSYYSFDV

SEQ ID NO: 312 - protein, 11 aa, Artificial Sequence
  Synthetic
RASQSINSHLA

SEQ ID NO: 313 - protein, 7 aa, Artificial Sequence
  Synthetic
AAINLQS

SEQ ID NO: 314 - protein, 9 aa, Artificial Sequence
  Synthetic
QQTNHYPLT

SEQ ID NO: 315 - protein, 10 aa, Artificial Sequence
  Synthetic
GFTFSSHAMH

SEQ ID NO: 316 - protein, 17 aa, Artificial Sequence
  Synthetic
SILSGGSETNYADSVKG

SEQ ID NO: 317 - protein, 12 aa, Artificial Sequence
  Synthetic
DTYEVTGNLLDY

SEQ ID NO: 318 - protein, 11 aa, Artificial Sequence
  Synthetic
RASQSVWSYLN

SEQ ID NO: 319 - protein, 7 aa, Artificial Sequence
  Synthetic
GASSLQS

SEQ ID NO: 320 - protein, 9 aa, Artificial Sequence
  Synthetic
MQSYSWPFT

SEQ ID NO: 321 - protein, 10 aa, Artificial Sequence
  Synthetic
GFTFNEYAMS

SEQ ID NO: 322 - protein, 17 aa, Artificial Sequence
  Synthetic
SIIPDGSETDYADSVKG

SEQ ID NO: 323 - protein, 11 aa, Artificial Sequence
  Synthetic
SLSSSGMHGDI

SEQ ID NO: 324 - protein, 11 aa, Artificial Sequence
  Synthetic
RASQSINNYLN

SEQ ID NO: 325 - protein, 7 aa, Artificial Sequence
  Synthetic
KASTLQS

SEQ ID NO: 326 - protein, 8 aa, Artificial Sequence
  Synthetic
HQYHSPYT

SEQ ID NO: 327 - protein, 10 aa, Artificial Sequence
  Synthetic
GFTFSDYGWH

SEQ ID NO: 328 - protein, 17 aa, Artificial Sequence
  Synthetic
IIESDGSGTYYADSVKG

SEQ ID NO: 329 - protein, 18 aa, Artificial Sequence
  Synthetic
NGEVGERGVRDYDYAMDY

SEQ ID NO: 330 - protein, 11 aa, Artificial Sequence
  Synthetic
RASQSINRYLN

SEQ ID NO: 331 - protein, 7 aa, Artificial Sequence
  Synthetic
ATSSLQS

SEQ ID NO: 332 - protein, 9 aa, Artificial Sequence
  Synthetic
QQYGSTPIT

SEQ ID NO: 333 - protein, 10 aa, Artificial Sequence
  Synthetic
GFTFTDYNVH

SEQ ID NO: 334 - protein, 17 aa, Artificial Sequence
  Synthetic
GINSSGSETNYADSVKG

SEQ ID NO: 335 - protein, 16 aa, Artificial Sequence
  Synthetic
DSVFKTVGGYDAVMDY

SEQ ID NO: 336 - protein, 11 aa, Artificial Sequence
  Synthetic
RASQSIYNYLA

SEQ ID NO: 337 - protein, 7 aa, Artificial Sequence
  Synthetic
GTSTLQS

SEQ ID NO: 338 - protein, 9 aa, Artificial Sequence
  Synthetic
MQSYSSPTT

SEQ ID NO: 339 - protein, 10 aa, Artificial Sequence
  Synthetic
GFTFKDYWLS

SEQ ID NO: 340 - protein, 17 aa, Artificial Sequence
  Synthetic
IINYGGSETYYADSVKG

SEQ ID NO: 341 - protein, 9 aa, Artificial Sequence
  Synthetic
TQTSYVMDY

SEQ ID NO: 342 - protein, 11 aa, Artificial Sequence
  Synthetic
RASQSVRSGLN

SEQ ID NO: 343 - protein, 7 aa, Artificial Sequence
  Synthetic
AASSLQS

SEQ ID NO: 344 - protein, 9 aa, Artificial Sequence
  Synthetic
MQSHSWPLT

SEQ ID NO: 345 - protein, 10 aa, Artificial Sequence
  Synthetic
GFTFSSGYMS

SEQ ID NO: 346 - protein, 17 aa, Artificial Sequence
  Synthetic
TISGSGRETNYADSVKG

SEQ ID NO: 347 - protein, 11 aa, Artificial Sequence
  Synthetic
DAWGGDSYFDP

SEQ ID NO: 348 - protein, 11 aa, Artificial Sequence
  Synthetic
RASQSIWSNLS

SEQ ID NO: 349 - protein, 7 aa, Artificial Sequence
  Synthetic
NASSLQS

SEQ ID NO: 350 - protein, 9 aa, Artificial Sequence
  Synthetic
QQYHGTPIT

SEQ ID NO: 351 - protein, 10 aa, Artificial Sequence
  Synthetic
GFTFTDYQMS

SEQ ID NO: 352 - protein, 17 aa, Artificial Sequence
  Synthetic
TIIWSGSETNYADSVKG

SEQ ID NO: 353 - protein, 7 aa, Artificial Sequence
  Synthetic
NKTPFDY

SEQ ID NO: 354 - protein, 11 aa, Artificial Sequence
  Synthetic
RASQSIRTHLA

SEQ ID NO: 355 - protein, 7 aa, Artificial Sequence
  Synthetic
DNSNLQS

SEQ ID NO: 356 - protein, 9 aa, Artificial Sequence
  Synthetic
QQYKGSPLT

SEQ ID NO: 357 - protein, 10 aa, Artificial Sequence
  Synthetic
GFTFTSYAMS

SEQ ID NO: 358 - protein, 17 aa, Artificial Sequence
  Synthetic
SISYSGGETDYADSVKG

SEQ ID NO: 359 - protein, 9 aa, Artificial Sequence
  Synthetic
SKGGLYFDY

SEQ ID NO: 360 - protein, 11 aa, Artificial Sequence
  Synthetic
RASQSISSYLA

SEQ ID NO: 361 - protein, 7 aa, Artificial Sequence
  Synthetic
GASSLQS

SEQ ID NO: 362 - protein, 9 aa, Artificial Sequence
  Synthetic
QQYGSTPET

SEQ ID NO: 363 - protein, 10 aa, Artificial Sequence
  Synthetic
GFTFSGYWIH

SEQ ID NO: 364 - protein, 17 aa, Artificial Sequence
  Synthetic
TISYSGDEAYYADSVKG

SEQ ID NO: 365 - protein, 11 aa, Artificial Sequence
  Synthetic
SPSDGDYGFDY

SEQ ID NO: 366 - protein, 11 aa, Artificial Sequence
  Synthetic
RASQSINNYLS

SEQ ID NO: 367 - protein, 7 aa, Artificial Sequence
  Synthetic
DGRILQS

SEQ ID NO: 368 - protein, 9 aa, Artificial Sequence
  Synthetic
MQYLAYPWT

SEQ ID NO: 369 - protein, 10 aa, Artificial Sequence
  Synthetic
GFTFGTYDMH

SEQ ID NO: 370 - protein, 17 aa, Artificial Sequence
  Synthetic
MISPSGGDTYYADSVKG

SEQ ID NO: 371 - protein, 11 aa, Artificial Sequence
  Synthetic
DSSGDWYAMAY

SEQ ID NO: 372 - protein, 11 aa, Artificial Sequence
  Synthetic
RASQSIRRYLA

SEQ ID NO: 373 - protein, 7 aa, Artificial Sequence
  Synthetic
GASNLQS

SEQ ID NO: 374 - protein, 9 aa, Artificial Sequence
  Synthetic
QHSSDTPLT

SEQ ID NO: 375 - protein, 10 aa, Artificial Sequence
  Synthetic
GFTFSDSAMG

SEQ ID NO: 376 - protein, 17 aa, Artificial Sequence
  Synthetic
IISYYGSETYYADSVKG

SEQ ID NO: 377 - protein, 11 aa, Artificial Sequence
  Synthetic
NPDGDLSALDY

SEQ ID NO: 378 - protein, 11 aa, Artificial Sequence
  Synthetic
RASQPISSYLN

SEQ ID NO: 379 - protein, 7 aa, Artificial Sequence
  Synthetic
GASSLQS

SEQ ID NO: 380 - protein, 9 aa, Artificial Sequence
  Synthetic
QQRLRSPFT

(19) United States
(12) Patent Application Publication
(10) Pub. No.: US 2024/0084017 A1
(43) Pub. Date: Mar. 14, 2024

(54) MONOCLONAL ANTIBODY AGAINST HUMAN MAC-1 AND USES THEREOF

(71) Applicants: Frank Wen-Chi LEE, Taipei (TW); Ascendo Biotechnology, Inc., Grand Cayman (KY)

(72) Inventors: Yen-Ta Lu, Taipei (TW); Chia-Ming Chang, Taipei (TW); Ping-Yen Huang, Taipei (TW); I-Fang Tsai, Taipei (TW); Frank Wen-Chi Lee, Orlando, FL (US)

(73) Assignee: Ascendo Biotechnology, Inc., Grand Cayman (KY)

(21) Appl. No.: 18/259,958

(22) PCT Filed: Dec. 30, 2021
(86) PCT No.: PCT/US2021/065802

§ 371 Date: Jun. 29, 2023

Related U.S. Application Data
(60) Provisional application No. 63/131,893, filed on Dec. 30, 2020.

(51) Int. Cl. C07K 16/28; A61P 35/00

(52) U.S. Cl. CPC C07K 16/2845; A61P 35/00; A61K 2039/505; C07K 2317/33; C07K 2317/73; C07K 2317/76

(57) ABSTRACT

Monoclonal antibodies against human Mac-1 are provided. These antibodies can bind to different states of Mac-1 so as to alter the biofunctions of Mac-1. These antibodies can modulate Th1/Th2 cytokine secretions by TLR-activated immune cells and can be used for the treatments of diseases related to acute and chronic inflammatory disorders, such as infectious diseases, and cancers.

### BACKGROUND OF INVENTION

Macrophage-1 antigen (Mac-1, integrin αMβ2) is mainly expressed on the surface of innate immune cells (including monocytes, neutrophils, NK cells, etc.). Mac-1 is a heterodimeric glycoprotein comprising non-covalently linked integrin αM (CD11b, CR3A, ITGAM) and integrin β2 (CD18, ITGB2). CD11b is a transmembrane protein with a large extracellular domain and a short cytoplasmic tail. Its extracellular domain comprises an I domain, a β-propeller domain, a thigh domain, a calf-1 domain, and a calf-2 domain. The I domain of CD11b has around 179 amino acids inserting into the β-propeller domain. This I domain is responsible for binding to promiscuous ligands (e.g., iC3b, fibrinogen, ICAM-1, CD40L, etc.) and participates in cell adhesion, migration, chemotaxis, and phagocytosis, and regulates inflammatory responses of innate immune cells.

Like other integrins, Mac-1 exists in distinct conformations with different ligand binding affinities. As shown in FIG. 1, CD11b and CD18 are bent in a V shape with the I-domain close to the membrane to form an inactive Mac-1 (low affinity). Inside-out signaling changes the Mac-1 to an open conformation, extending the I domain away from the membrane for optimal ligand binding. One epitope located on the I-EGF2 of CD18 is hidden in the bent conformation (inactive or closed state); this epitope becomes exposed and can be recognized by a monoclonal antibody (KIM127) in the extended or open state (J. Immunol. 2001; 166: 5629-5637). This conformational change also results in the rearrangement of the I domain site such that it becomes a high affinity site for ligand binding and forms an epitope for mAb m24 binding (Proc. Natl. Acad. Sci. USA. 2004; 101: 2333-2338). Such conformational changes accompanying ligand binding affinity changes are tied to Mac-1 functions.

### SUMMARY OF THE INVENTION

Embodiments of the invention relate to antibodies that can bind specifically to Mac-1 and modulate immune cell functions. These antibodies may be used to treat various Mac-1 associated diseases or conditions, such as infectious diseases or cancers.

One aspect of the invention relates to antibodies against human Mac-1. An antibody against human Mac-1 in accordance with one embodiment of the invention comprises a light-chain variable region sequence and a heavy-chain variable region sequence selected from SEQ ID NO:1 through SEQ ID NO:158 shown in Table I.

One aspect of the invention relates to methods for treating a disorder associate with Mac-1. A method in accordance with one embodiment of the invention comprises administering to a subject in need thereof an effective amount of an antibody of the invention. The disorder is an acute or chronic inflammation. The disorder may be an infection or a cancer.

Other aspects of the invention would become apparent from the following description and the associated drawings.

### BRIEF DESCRIPTION OF THE DRAWINGS

FIG. 1 shows the two conformations of Mac-1 and their epitopes for activation-sensitive mAbs.

FIG. 2 shows results of characterization of HEK293/Mac-1 using various antibodies. HEK293 cells were incubated in PBS (Mock) or PBS/MnCl₂ (Mn²⁺). Bindings of isotype control IgG, a CD11b specific mAb (clone ICRF44), a CD18 specific mAb (clone 6.7), or β2 activation-dependent mAbs (KIM127 and m24) were detected using flow cytometry.

FIG. 3A shows that representative anti-Mac-1 antibodies of the invention (DF3M-5, H4L2, m2396, 24G05, and 28E07-HH) predominantly bind to myeloid immune cells (monocytes and neutrophils). Other antibodies of the invention show similar properties.

FIG. 3B shows that clones m2396, DF3M-5, and 24G05 bind to mouse Mac-1 expressing cell line Raw264.7.

FIG. 4 shows examples of anti-Mac-1 antibodies that can modulate conformational changes of Mac-1 under PBS (Mock) or PBS/MnCl₂ (Mn²⁺) conditions.

FIG. 5 shows that anti-Mac-1 antibody treatments can modulate TLR4 agonist-induced Th1/Th2 cytokines responses in mice in vivo. Data are shown as the means±SEM (4 mice per group).

FIG. 6 shows that anti-Mac-1 antibodies reduce tumor growths in A549 human lung tumor bearing humanized NOG-EXL mouse model in vivo. Data are shown as the means±SEM (10 mice per group).

FIG. 7A shows that anti-Mac-1 antibody enhanced the expression of functional markers in myeloid cells isolated from HIV patients.

FIG. 7B shows that anti-Mac-1 antibody reduced the virus load in PBMCs from HIV patients.

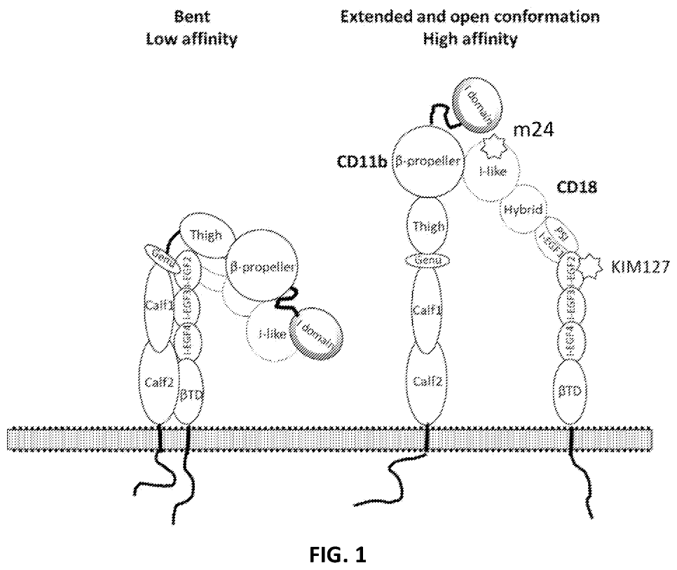

### DETAILED DESCRIPTION

Embodiments of the invention relate to antibodies that can bind specifically to Mac-1 and modulate immune cell functions. These antibodies may be used to treat various Mac-1 associated diseases or conditions, such as infectious diseases or cancers.

Human antibody and mouse antibody phage display libraries were constructed and screened to isolate clones carrying specific antibody genes that can recognize Mac-1. These anti-Mac-1 antibodies are shown to bind Mac-1 on the HEK293/Mac-1 cells and innate immune cells. These antibodies can selectively bind to different states of Mac-1 (bent or extended/open conformation) and modulate the conformational changes of Mac-1. These anti-Mac-1 antibodies are shown to modulate TLR-induced cytokine productions and therefore can be used to treat acute and chronic inflammatory disorders, such as infectious diseases (ref: WO 2020/033929 A1) and cancers (ref: WO 2019/177669 A1 and WO 2016/197974 A1).

The following describes specific examples of various aspects of the invention. One skilled in the art would appreciate that these specific examples are for illustration only and that other modifications and variations are possible without departing from the scope of the invention.

### Material and Method

### Reagents and Antibodies

The antibodies and reagents used for flow cytometry are KIM127 (hybridoma from ATCC), m24-PE (BioLegend), anti-CD11b-APC (clone ICRF44, BioLegend), anti-CD18-APC (clone 6.7, BD), BSA (BioShop), Rat IgGlκ-APC (BioLegend), and Rat IgGlκ-PE (BioLegend). The KIM127 antibody and BSA were conjugated with CF647, i.e., labeled with CF647 labeling kit (CF Dye & Biotin SE Protein Labeling Kits, Biotium).

### Cell Culture and Stable Transfection

Stable transfection of human integrin Mac-1 in HEK293 cells (BCRC) was performed using jetPRIME® (PolyPlus) transfection protocols. Briefly, HEK293 cells were cultured in Dulbecco's Modified Eagle's Medium (DMEM, Corning), supplemented with 10% heat-inactivated fetal bovine serum (Gibco) and 50 IU/mL penicillin and streptomycin (Corning) at 37° C. The cells were seeded at 8×10⁵ cells/well on a 6-well plate (Coster). Next day, a mixture of the jetPRIME® reagent and 2 μg pcDNA3.1/human CD18 expression plasmid carrying a hygromycin-resistance gene was added to the cells, and the cells were cultured for 24 hr. The selection antibiotic, hygromycin B (InvivoGen), was added at a concentration of 200 μg/ml, and half of the culture media containing the antibiotic were changed every 2 to 3 days. After 3 weeks, the CD18-expression cells were collected using the Cell Sorter (SH800Z, SONY) to pick up CD18-high expression cells and seeded at single cell/well and 5000 cells/well on a 24-well plate (Coster) and a 6-well plate. Cells were maintained in DMEM medium with 10% heat-inactivated fetal bovine serum, 50 IU/mL penicillin and streptomycin, and 200 μg/ml Hygromycin B at 37° C. After the cells were enriched, human CD18 expression was analyzed with anti-human CD18-APC (clone 6.7, BD) antibody by flow cytometry. The permanent HEK293/human CD18 cells (clone 2B4) were seeded at 6×10⁵ cells/well on a 6-well plate. The transfection protocol for human CD11b was the same as above. Briefly, 2 μg pcDNA3.1/human CD11b plasmid was mixed with jetPRIME® reagent and added to cells. Next days, the selection antibiotics, 1 mg/ml G418 (InvivoGen) and 200 μg/ml Hygromycin B, were added, and the medium containing selection antibiotics was changed every 2 to 3 days. The Mac-1 expression was measured with anti-human CD11b-APC (clone ICRF44, BioLegend) and anti-hCD18-APC by flow cytometry. The stable clone 1-4 was picked up.

### Flow Cytometry

The HEK293/Mac-1 cells (clone1-4) were counted and washed twice with staining buffer (PBS containing 1% FBS, and 0.1% sodium azide). Cells were adjusted at a concentration of 1×10⁵ cells/ml in staining buffer and treated with/without 0.25 mM MnCl₂ (Sigma). Cells were treated with anti-Mac-1 antibodies and fluorescent conjugated anti-human IgG4 antibodies and incubated for 15 mins. After washing with the staining buffer, the cells were analyzed by flow cytometry. In some examples, these cells were treated with the antibodies (ICRF44, KIM127, m24, or isotype control) in the presence or absence 10 μg/ml anti-Mac-1 antibodies. The cells were then incubated at 37° C. for 30 min. After washing with the staining buffer, the cells were analyzed by flow cytometry.

### Cytokine Measurement

Balb/c mice (n=4/group) were intraperitoneally injected with 5 mg/kg LPS and 10 mg/kg anti-Mac-1 antibodies for 4 hours. Murine Th1 and Th2 cytokines in the serum were detected by ProcartaPlex MS (Thermo Fisher Scientific) according to the manufacturer instructions.

### Protocol of Cancer Treatment

Human umbilical cord blood derived CD34+ cells were transplanted into NOG-EXL mice via the tail vein. About 10 weeks after transplantation, peripheral blood were collected from the humanized NOG-EXL animals under anesthesia and used for FACS analysis. The types, proportions, fluorescence intensities, and absolute counts of immune cells (T cells, B cells, dendritic cells, and monocyte cells) were analyzed. When the average hCD45⁺%>15%, hCD3⁺ of hCD45⁺%>3%, and hCD14⁺ of hCD45⁺%>5%, the humanized NOG-EXL animals were used for the anti-cancer study.

Human lung cancer A549 cells were cultured in a 37° C. incubator containing 5% CO₂ with 10% FBS in F-12K medium. The cells were sub-cultured within 10 passages before being inoculated into mice. A549 cells (5×10⁶ cells) were mixed with Matrigel (v/v 1:1) in a volume of 200 μl immediately before injection subcutaneously. Before inoculation, mice were anesthetized with 2-5% isoflurane.

When tumor volumes reached 20-50 mm³, tumor-bearing animals were grouped into 3 groups based on the frequency of macrophage in human CD45⁺ cells, the frequency of CD3⁺ cell in human CD45⁺ cells, and tumor volumes, each group contains 10 mice. The day of grouping was denoted as day 0. Mice were treated on day 0.

Tumor volume: The tumor volume was calculated as follows: V=(length×width²)/2. Tumor volume was measured and recorded twice a week during inoculation, grouping, and during the dose period. Tumor growth inhibition (TGI) was calculated as follows: TGI=(1−(T/C))×100%; T and C as the mean tumor volumes of the treatment and control groups, respectively, on the measurement day.

### Protocol of Infectious Disease Treatment

### PBMC Isolation from HIV Patients

Fifteen HIV-1 infected patients receiving regular highly active antiretroviral therapy (ART) treatments with undetectable plasma viral load (<50 HIV-1 RNA copies/ml) and countable CD4 cells (count>200/mm³) were recruited at National Taiwan University Hospital (Taipei, Taiwan). The clinical and laboratory data were collected and acquired from medical records. Each blood sample was processed within 24 hours after collection, and leukocytes were isolated for further examination. This study was approved by the Institutional Review Board of National Taiwan University Hospital (Taipei, Taiwan), and written informed consents were obtained from each participant.

Peripheral blood mononuclear cells (PBMC) were isolated from whole blood samples by means of Ficoll-Paque (Amersham Biosciences, Sweden) gradient centrifugation. Cells were cultured in 96-well U-bottom culture plates (2×10⁵ cells/well) and resuspended in RPMI-1640 medium with 10% fetal bovine serum (FBS), 100 nM elvitegravir (Cayman), and 100 nM efavirenz (Cayman) in the presence of human IgG4 antibody (BioLegend) or anti-Mac-1 antibody (clone H4L2) 10 μg/ml for 3 days.

### Functional Marker Detection of PBMCs of HIV Patients

Cell suspensions were incubated with Fc blocker (BD Bioscience) in PBS containing 1% FBS and 0.1% sodium azide before staining with fluorochrome-labeled antibodies. Antibodies against CD11b (clone ICRF44, BioLegend), CD86 (clone 2331, BioLegend), HLA-DR (clone L243, BioLegend), and CD80 (clone L307, BD) were used for marker staining. FVS786 viability staining was used to exclude dead cells from analysis. The mean fluorescence intensity of stained cells was measured by CytoFlex flow cytometry and analyzed by Kaluza software (Beckman Coulter).

### Intracellular HIV Virus Detection—2 Long-Terminal Repeat (LTR)—DNA Circles Quantitation

DNA of 3 day-cultured PBMC (3×10⁶ cells/well in a 24-well culture plate) treated with/without PMA (100 ng/ml) and ionomycin (1 μg/ml) in the presence of human IgG4 antibody (BioLegend) or Anti-Mac-1 antibody (H4L2, 10 μg/ml) was extracted with QIAamp DNA Blood Mini Kit (Qiagen, MD, USA) and DNA were eluted by 50 μl nuclease-free water. Digital PCR was performed with the QX100™ Droplet Digital™ PCR platform (Bio-Rad, Hercules, California). The ddPCR mix was made by adding 1-5 μl of sample to 10 μl 2× ddPCR™ supermix for probes (Bio-Rad), 1 μl EcoR, 500 nM of primers, and 250 nM of probe in a final volume of 20 μl. The mix was placed in an 8-channel cartridge, 70 μl of droplet generating oil (Bio-Rad) was added and droplets were generated in the QX100™ droplet generator (Bio-Rad). Droplet in oil suspensions were transferred to an ddPCR 96-well plate (Bio-Rad) and PCR was performed in the T100™ Thermal Cycler (Bio-Rad). DdPCR amplification reactions consisted of an initial denaturation at 95° C. for 10 min, followed by 40 cycles of 95° C. for 15 sec denaturation and 60° C. for 60 sec annealing/elongation temperature, and enzyme deactivation at 98° C. for 10 min. The ramping rate of each step is 2° C./sec. The sequences of primer pairs are listed in Table VI.

### Statistical Analysis

Results were compared by Fisher's exact test for categorical variables and paired t test or unpaired t test for continuous variables as appropriate. Data are reported as the mean±SEM. Statistical analysis was performed using Prism 9.0 software. Two-sided tests were used, and a p-value of <0.05 was considered statistically significant.

### Results

### Expression of Heterodimeric CD11b/CD18 (Mac-1) on HEK293 Cell Surface

HEK293 cells, which do not express endogenous Mac-1, were transfected with pcDNA3.1/human CD11b and pcDNA3.1/human CD18 plasmids using liposome transfections. After G418 and hygromycin selections, we obtained several single-cell clones stably expressing the human Mac-1 on the cell surface by FACS sorting using CD18-specific mAb (clone 6.7) and CD11b specific mAb (clone ICRF44). One clone, designated 1-4, was selected for all the examples presented in this description. Other clones show similar properties.

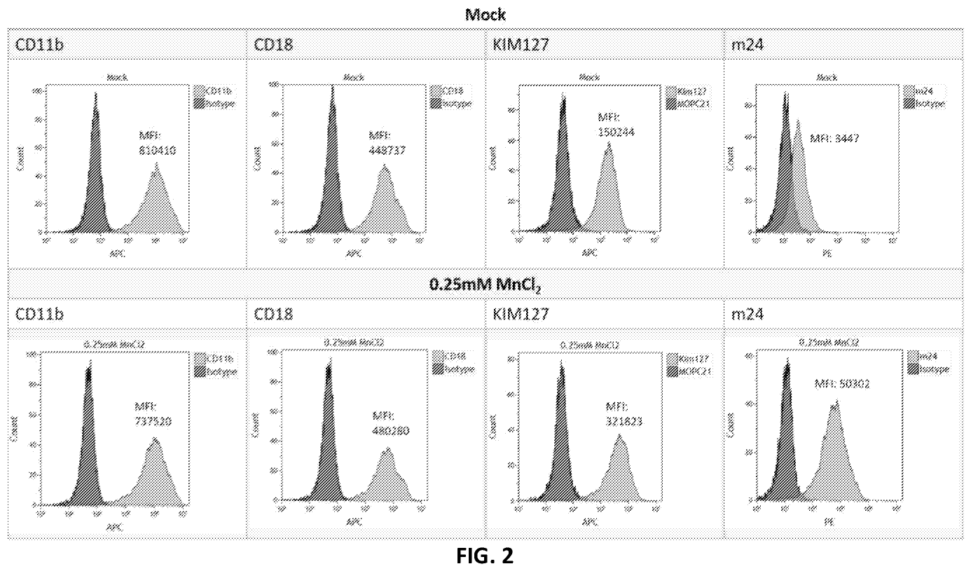

The expressions of CD11b and CD18 on the HEK293 cells, as detected by flow cytometry, are shown in FIG. 2. We verified the conformational state on the surface of HEK293/Mac-1 cells using two activation-sensitive antibodies, mAbs, KIM127 and m24. KIM127 can fully bind to HEK293/Mac-1, suggesting that Mac-1 is in the extended conformation. Little binding of m24 to HEK293/Mac-1 cells were observed in PBS (Mock), suggesting that Mac-1 is in the low affinity state. The HEK293/Mac-1 in the PBS is partially activated. In contrast, Mn²⁺ treatment induced 100% of Mac-1 molecule to adopt an extended, high-affinity conformation. Thus, these cells provide an excellent platform for the screening of monoclonal antibodies of human Mac-1.

### Anti-Mac-1 Antibodies Selectively Bind to the Different States of Mac-1 on HEK293/Mac-1 Cell Surface

We constructed human antibody and mouse antibody phage display libraries and then screened and isolated clones carrying specific antibody genes that can recognize Mac-1. A total of 79 clones were picked from the phage pools from each round of selection. The amino acid sequences of the variable regions of these clones are listed in Table I and Table VII. To verify that these clones can bind to Mac-1 in its native conformation, we generated recombinant antibodies from these clones with human IgG4 backbone. The recombinant anti-Mac-1 antibodies were used in flow cytometric analysis of HEK293/Mac-1 cells. As listed in Table II, these antibodies can indeed recognize Mac-1 on the surface of HEK293/Mac-1 cells.

Conformational change of Mac-1 is involved in the regulation of its functions. We examine whether these antibodies can recognize different conformations of Mac-1. As listed in Table II, some clones selectively bind to an activation-specific epitope on Mac-1 molecules on HEK293/Mac-1 cells after stimulation with Mn²⁺ (Mock/MnCl₂ Ratio<1). In contrast, some clones preferentially recognize the resting form of Mac-1 (Mock/MnCl₂ Ratio>1). The deduced amino-acid sequences of the CDRs and framework regions of selected clones are shown in Table I.

### Anti-Mac-1 Antibodies Predominantly Bind to Mac-1 on the Innate Immune Cell Surface

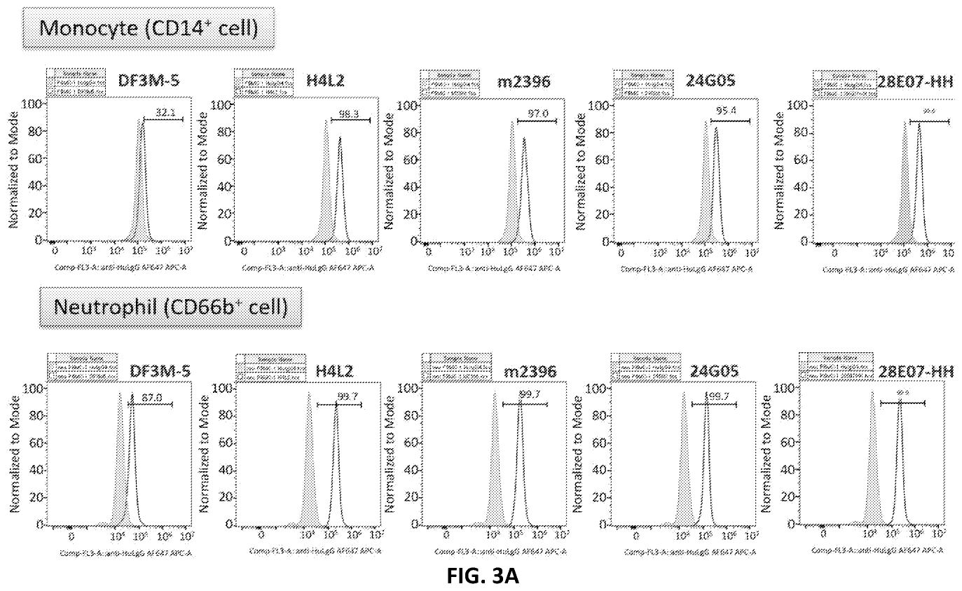

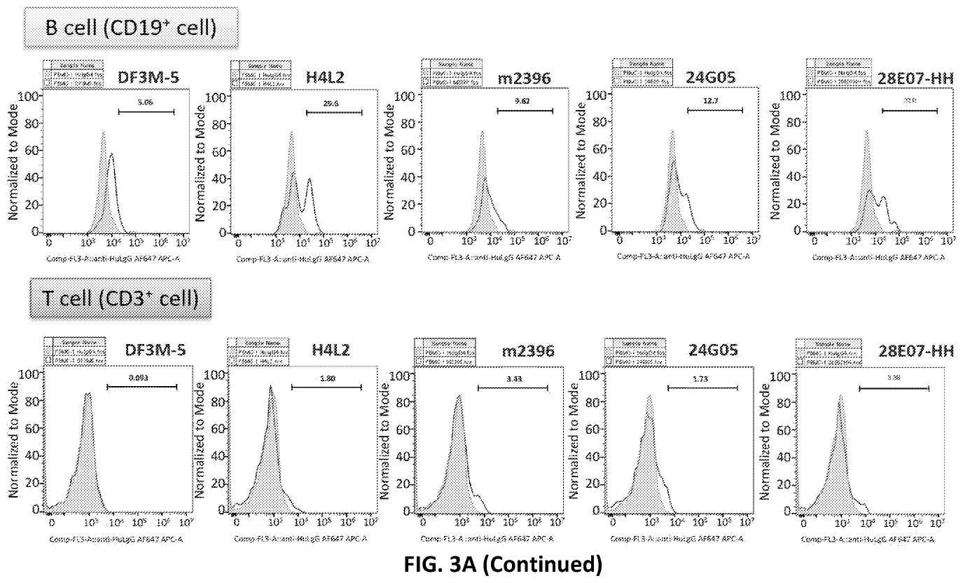

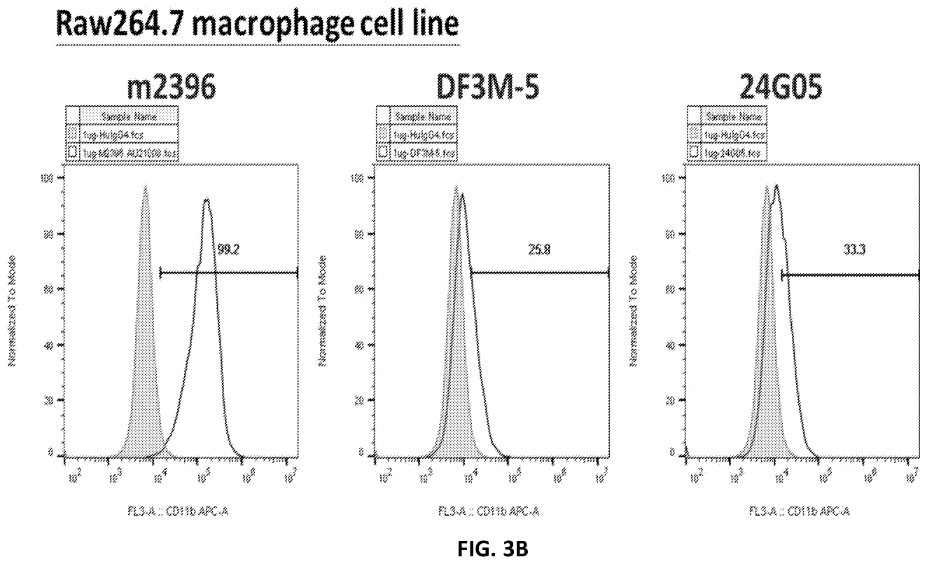

The innate immune cells such as monocytes (CD14⁺ cells) and neutrophils (CD66b⁺ cells) are the main cells that express Mac-1 on their cell surface. Some populations of B cells also expressed Mac-1 on their cell surface (Proc Natl Acad Sci U S A. 2008 Apr 1;105(13):5195-200). The specificities of selective anti-Mac-1 antibodies were determined by flow cytometry using human whole blood. As shown in FIG. 3A, anti-Mac-1 antibodies in this example were able to bind to the innate immune cells (CD14⁺ and CD66b⁺ cells) and small populations of B cells (CD19⁺ cells). In contrast, these antibodies did not bind to T cells (CD3⁺ lymphocytes). Taken together, these results indicate that anti-Mac-1 antibodies can specifically bind to the Mac-1 epitope on the immune cells. To determine whether an anti-Mac-1 antibody validated for human Mac-1 will cross-react with the mouse Mac-1, we use Raw 264.7 mouse macrophage cell line that expressed mouse Mac-1 on the cell surface. As shown in FIG. 3B, some clones, such as DF3M-5, m2396, and 24G05, can bind to the surface of Raw 264.7, suggesting that these clones can cross react with mouse Mac-1.

### Anti-Mac-1 Antibodies Induce a Conformational Change in Mac-1

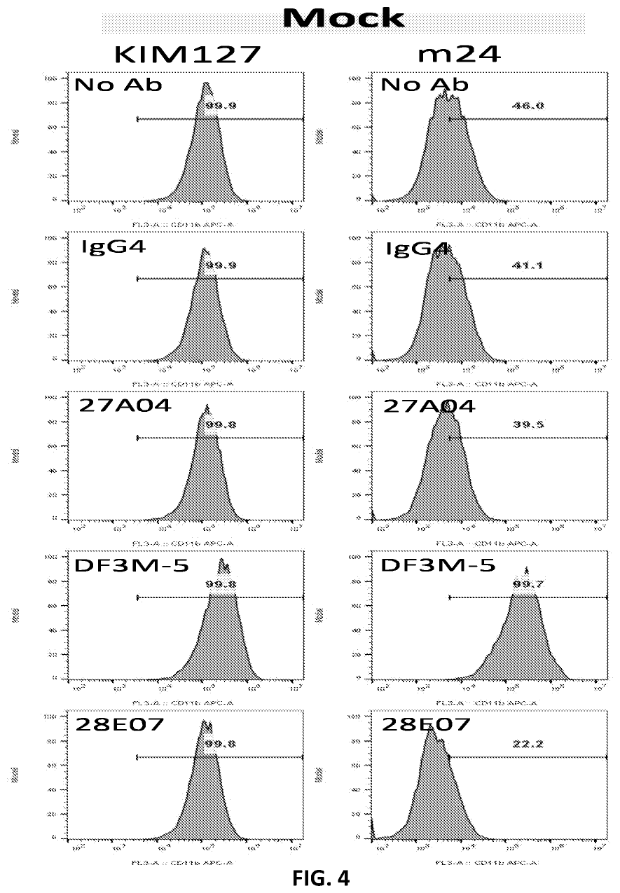

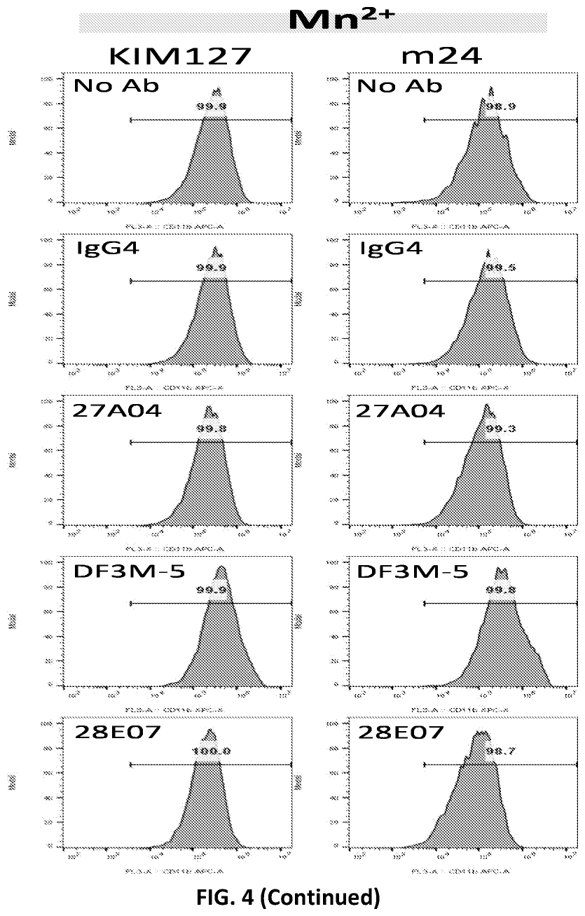

It is well known that inside-out signaling induces global conformational changes of Mac-1 leading to outside-in signaling. To screen which anti-Mac-1 antibodies would induce conformational changes in Mac-1, we used KIM127 and m24 antibodies, which bind preferentially to Mac-1 in the extended conformation, as reporters to detect conformational changes. As shown in FIG. 4 left panel, incubation of the antibodies with HEK293/Mac-1 cells in PBS buffer (Mock) resulted in basal levels of KIM127 and m24 bindings. In contrast, incubation of the antibodies with HEK293/Mac-1 cells in MnCl₂/PBS buffer (Mn²⁺) induced maximal levels of KIM127 and m24 bindings (FIG. 4 right panel). Incubation with DF3M-5 in the Mock condition induced a small increase in KIM127 binding and a large increase in m24 binding (FIG. 4 left panel), indicating that this DF3M-5 clone can serve as an agonist to enhance the conformational change of Mac-1. Other clones that can serve as agonists (based on m24 expression relative IgG4 control >1) are listed in Table III. In contrast, incubation with 28E07 in the Mn²⁺ condition induces a small decrease in KIM127 binding and a large decrease in m24 binding (FIG. 4 right), indicating that this 28E07 clone can serve as an antagonist to reduce the conformational change of Mac-1. Other clones that can serve as antagonists (based on m24 expression relative IgG4 control <1) are listed in Table IV. Results from these studies indicate that antibodies of the invention may be selectively used to control the conformational changes of Mac-1, thereby regulating the functions of Mac-1.

### Anti-Mac-1 Antibodies Modulate Th1/Th2 Cytokine Secretion by TLR-Activated Immune Cells In Vivo.

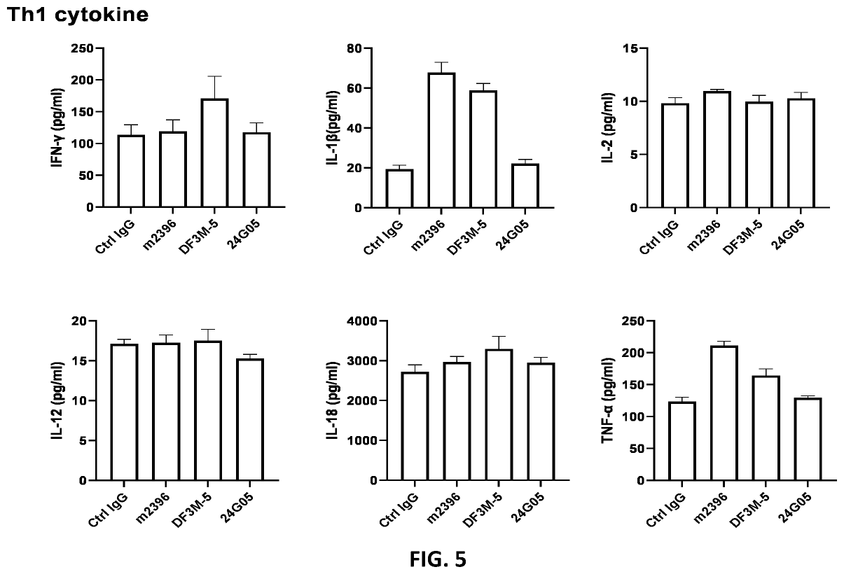

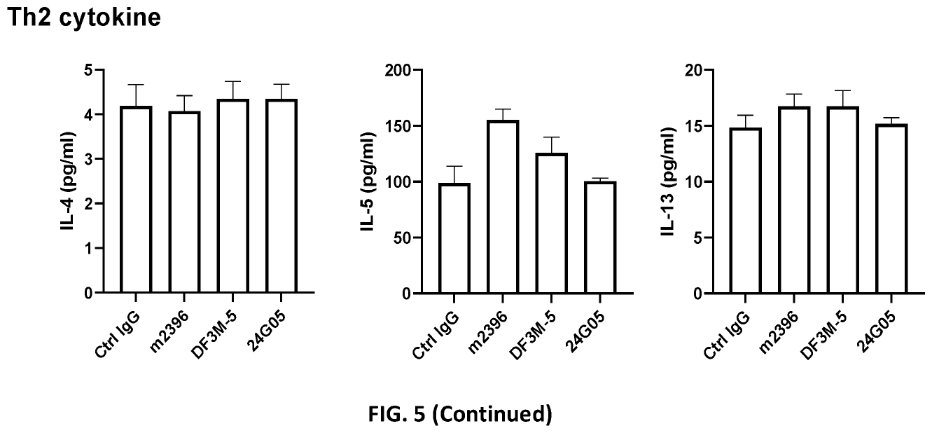

Previous studies show that active CD11b integrin engages in crosstalks with the MyD88 and TRIF pathways and modulate TLR signaling in innate immune responses (Nat Immunol. 2010 Aug;11(8):734-42). To examine whether anti-Mac-1 antibodies of the invention can modulate Th1/Th2 cytokine secretions in TLR-activated immune cells in vivo, Balb/c mice (n=4/group) were intraperitoneal injected with 5 mg/kg LPS and 10 mg/kg anti-Mac-1 antibodies. Four hours later, serum Th1/Th2 cytokines were measured by ProcartaPlex™ MS. As shown in FIG. 5, m2396 and DF3M-5 treatments enhanced TLR4-induced Th1 cytokines (such as IFN-γ, IL-1β, and TNF-α) and slightly enhance TLR4-induced Th2 cytokines (such as IL-5 and IL-13) in the serum. These results suggest that anti-Mac-1 antibody (clones m2396 and DF3M-5) treatment can skew the TLR-induced Th1/Th2 responses. In contrast, 24G05 treatment didn't alter Th1/Th2 cytokine profile.

### Anti-Mac-1 Antibody Treatment Reduces Tumor Growth

The anti-cancer activities of the anti-Mac-1 antibodies of the invention (e.g., m2396 and 28E07-HH) were further evaluated in the treatment of A549 cancer model in female NOG-EXL humanized mice.

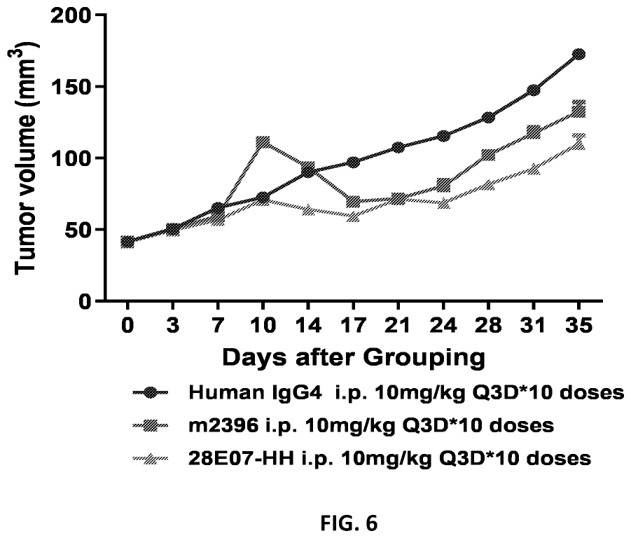

When the average tumor volumes reached about 41 mm³, tumor bearing mice were randomized into 3 groups (Human IgG4, m2396, and 28E07-HH) and the treatments were started. The mean tumor sizes of mice reached 172.59 mm³ in Human IgG4 group, 132.51 mm³ in m2396 group, and 109.88 mm³ in 28E07-HH group on Day 35 post grouping (FIG. 6). FIG. 6 shows results from representative antibodies m2396 and 28E07-HH. Other antibodies of the invention have similar properties. The tumor growth inhibition (TGI) % of the m2396 group and 28E07-HH group were 23.59%, and 35.93%, respectively. The TGI of the different groups at different time points were shown in Table V. These results indicate that these anti-Mac-1 antibodies can serve as therapeutic antibodies to treat human cancer.

### Anti-Mac-1 Antibody Treatment Reduced HIV Viral Load and Reverses Immunosuppressed Phenotype of PBMC in HIV Patients

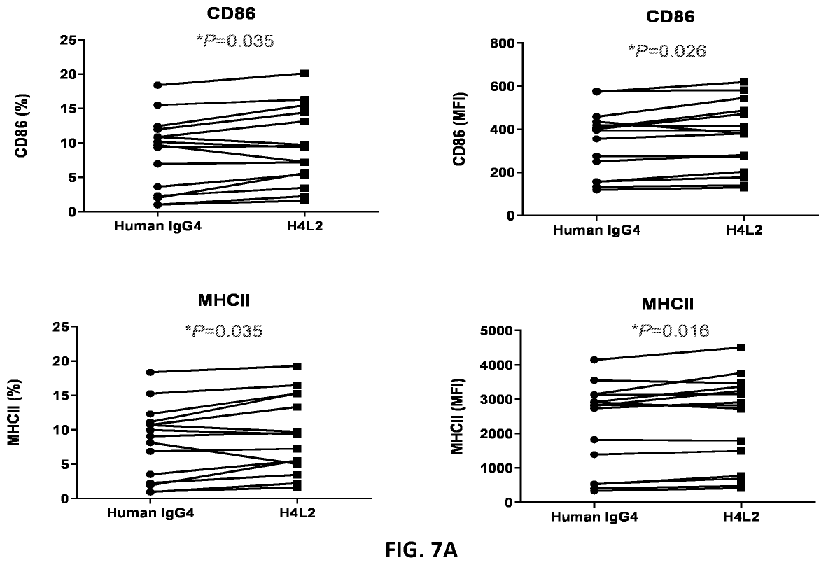

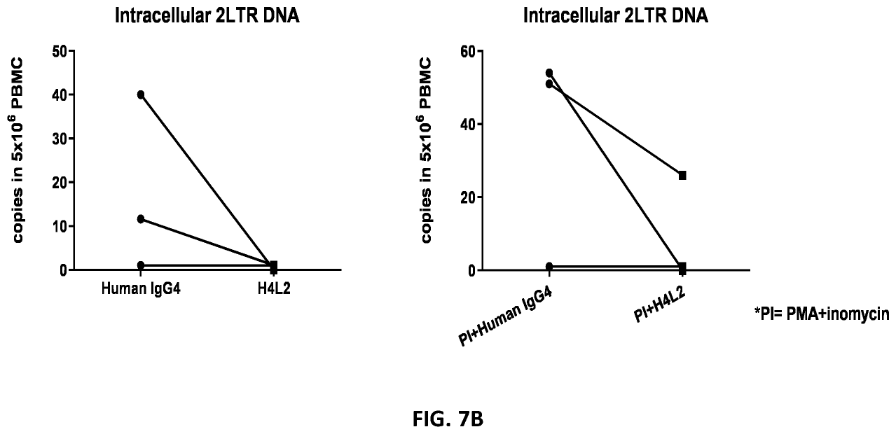

To test the efficacy of the anti-Mac-1 antibody-mediated inhibitory actions against HIV, PBMC isolated from fifteen latent HIV-infected patients were treated with anti-Mac-1 antibodies for 3 days in vitro. As shown in FIG. 7A, the anti-Mac-1 antibody (H4L2 shown as a representative of anti-Mac-1 antibodies) significantly enhanced the expression of CD86 and MHC class II functional markers in myeloid cells of HIV patients. These results indicate that enhanced T cell activation and dendritic cells (DCs) maturation in HIV patients can be achieved with the anti-Mac-1 antibodies of the present invention. These enhanced immune responses may suggest a potential application for the antibodies of the invention in the treatment of HIV infection.

While combination antiretroviral therapy (ART) may suppress HIV replication. HIV-1 persists in the infected cells as a stable integrated genome and more labile unintegrated DNA, which includes linear, 1-LTR and 2-LTR circular DNA. 2-LTR circle DNA, although less abundant, is considered a surrogate marker for recent infection events and is currently used as a diagnostic tool. (C. Orlandi et al., “A comparative analysis of unintegrated HIV-1 DNA measurement as a potential biomarker of the cellular reservoir in the blood of patients controlling and non-controlling viral replication,” J. Transl. Med. 18, 204 (2020). Doi: 10.1186/s12967-020-02368-y).

To detect the load of intracellular HIV virus, HIV virus DNA reservoir was quantified using the 2 long-terminal repeat (LTR) DNA circles as the marker. Because these fifteen HIV-1 infected patients were receiving regular highly active antiretroviral therapy (ART) treatments, only 3 of the 15 patients had detectable levels of the HIV DNA by the LTR assay. Nevertheless, declines in the HIV 2LTR DNA levels were observed in these 3 patients' PBMC samples treated with the anti-Mac-1 antibody or with the anti-Mac-1 antibody in combination with phorbol 12-myristate 13-acetate (PMA) and ionomycin (FIG. 7B).

While the invention has been described with a limited number of examples, one skilled in the art would appreciate that these examples are for illustration only and that other modifications and variations are possible without departing from the scope of the invention. Therefore, the scope of protection should only be limited by the attached claims.

### Tables

TABLE I
Heavy-chain variable region sequences and
light-chain variable region sequences of
SEQ ID NO: 1 through SEQ ID NO: 158

| | | |
|---|---|---|
| SEQ ID NO | Description | Sequence |

Anti-Mac-1 antibody sequence clone: 24F08
(Underline is a CDR sequence)

| | | |
|---|---|---|
| SEQ ID | Heavy chain | EVQLVESGGGLVQPGGSLRLSCAASGFTFSSYWMSWV |
| NO: 1 | variable | RQAPGKGLEWVSIINYSGREADYADSVKGRFTISRDN |
| | | SKNTLYLQMNSLRAEDTAVYYCARDGSYVGQAHEAFD |
| | | IWGQGTLVTVSS |
| SEQ ID | Light chain | DIQMTQSPSSLSASVGDRVTITCRASQSISKYLAWYQ |
| NO: 2 | variable | QKPGKAPKLLIYGTSNLQSGVPSRFSGSGSGTDFTLT |
| | | ISSLQPEDFATYYCQQSRSWPLTFGQGTKVEIK |

Anti-Mac-1 antibody sequence clone: 24F09
(Underline is a CDR sequence)

| | | |
|---|---|---|
| SEQ ID | Heavy chain | EVQLVESGGGLVQPGGSLRLSCAASGFTFSSAWMSWV |
| NO: 3 | variable | RQAPGKGLEWVSTIYWSGSEINYADSVKGRFTISRDN |
| | | SKNTLYLQMNSLRAEDTAVYYCARSFASGESAMDVWG |
| | | QGTLVTVSS |
| SEQ ID | Light chain | DIQMTQSPSSLSASVGDRVTITCRASQSISNYLAWYQ |
| NO: 4 | variable | QKPGKAPKLLIYDASNLQSGVPSRFSGSGSGTDFTLT |
| | | ISSLQPEDFATYYCQQYYSSPPTFGQGTKVEIK |

Anti-Mac-1 antibody sequence clone: 24F11
(Underline is a CDR sequence)

| | | |
|---|---|---|
| SEQ ID | Heavy chain | EVQLVESGGGLVQPGGSLRLSCAASGFTFSTSWMHWV |
| NO: 5 | variable | RQAPGKGLEWVSIINSGGGEAYYADSVKGRFTISRDN |
| | | SKNTLYLQMNSLRAEDTAVYYCARGDAAFDYWGQGTL |
| | | VTVSS |
| SEQ ID | Light chain | DIQMTQSPSSLSASVGDRVTITCRASQLIRKKLAWYQ |
| NO: 6 | variable | QKPGKAPKLLIYAASTLQSGVPSRFSGSGSGTDFTLT |
| | | ISSLQPEDFATYYCMQSGSPQYTFGQGTKVEIK |

Anti-Mac-1 antibody sequence clone: 24F12
(Underline is a CDR sequence)

| | | |
|---|---|---|
| SEQ ID | Heavy chain | EVQLVESGGGLVQPGGSLRLSCAASGFTFTNYWMGWV |
| NO: 7 | variable | RQAPGKGLEWVSIIISDGGEIIYADSVKGRFTISRDN |
| | | SKNTLYLQMNSLRAEDTAVYYCARIHAGTGSSADYWG |
| | | QGTLVTVSS |
| SEQ ID | Light chain | DIQMTQSPSSLSASVGDRVTITCRASQSIYNYLNWYQ |
| NO: 8 | variable | QKPGKAPKLLIYDASTLQSGVPSRFSGSGSGTDFTLT |
| | | ISSLQPEDFATYYCQQYYSYPWTFGQGTKVEIK |

Anti-Mac-1 antibody sequence clone: 24G01
(Underline is a CDR sequence)

| | | |
|---|---|---|
| SEQ ID | Heavy chain | EVQLVESGGGLVQPGGSLRLSCAASGFTFRTFGMNWV |
| NO: 9 | variable | RQAPGKGLEWVSGIVPSGSEIDYADSVKGRFTISRDN |
| | | SKNTLYLQMNSLRAEDTAVYYCARDHSHYTGPFDVWG |
| | | QGTLVTVSS |
| SEQ ID | Light chain | DIQMTQSPSSLSASVGDRVTITCRASQSIYSYLNWYQ |
| NO: 10 | variable | QKPGKAPKLLIYGASILQYGVPSRFSGSGSGTDFTLT |
| | | ISSLQPEDFATYYCHQSNSSPGTFGQGTKVEIK |

Anti-Mac-1 antibody sequence clone: 24G05
(Underline is a CDR sequence)

| | | |
|---|---|---|
| SEQ ID | Heavy chain | EVQLVESGGGLVQPGGSLRLSCAASGFTFTSYWMTWV |
| NO: 11 | variable | RQAPGKGLEWVSTIVGGGGEADYADSVKGRFTISRDN |
| | | SKNTLYLQMNSLRAEDTAVYYCARDYVADNHGAMDYW |
| | | GQGTLVTVSS |
| SEQ ID | Light chain | DIQMTQSPSSLSASVGDRVTITCRASQGLSSYLNWYQ |
| NO: 12 | variable | QKPGKAPKLLIYGMSTLQSGVPSRFSGSGSGTDFTLT |
| | | ISSLQPEDFATYYCMQYYHWPYTFGQGTKVEIK |

Anti-Mac-1 antibody sequence clone: 24G07
(Underline is a CDR sequence)

| | | |
|---|---|---|
| SEQ ID | Heavy chain | EVQLVESGGGLVQPGGSLRLSCAASGFTFSDYIMHWV |
| NO: 13 | variable | RQAPGKGLEWVSAISPSGSEIYYADSVKGRFTISRDN |
| | | SKNTLYLQMNSLRAEDTAVYYCARNAWDNNWVREYGM |
| | | DYWGQGTLVTVSS |
| SEQ ID | Light chain | DIQMTQSPSSLSASVGDRVTITCRASQSGNNNLAWYQ |
| NO: 14 | variable | QKPGKAPKLLIYGASTLQSGVPSRFSGSGSGTDFTLT |
| | | ISSLQPEDFATYYCMQSNSYPLTFGQGTKVEIK |

Anti-Mac-1 antibody sequence clone: 24G08
(Underline is a CDR sequence)

| | | |
|---|---|---|
| SEQ ID | Heavy chain | EVQLVESGGGLVQPGGSLRLSCAASGFTFSDYKIHWV |
| NO: 15 | variable | RQAPGKGLEWVSIYADSVKGRFTISRDNSKNTLYLQM |
| | | NSLRAEDTAVYYCARSSYGEGYAFDYWGQGTLFTVSS |
| SEQ ID | Light chain | DIQMTQSPSSLSASVGDRVTITCRASQDVDSYLNWYQ |
| NO: 16 | variable | QKPGKAPKLLIYDAISLQSGVPSRFSGSGSGTDFTLT |
| | | ISSLQPEDFATYYCQQYYSLPLTFGQGTKVEIK |

Anti-Mac-1 antibody sequence clone: 24G09
(Underline is a CDR sequence)

| | | |
|---|---|---|
| SEQ ID | Heavy chain | EVQLVESGGGLVQPGGSLRLSCAASGFTFSDYAIGWV |
| NO: 17 | variable | RQAPGKGLEWVSTIYWSGSNAYYADSVKGRFTISRDN |
| | | SKNTLYLQMNSLRAEDTAVYYCARLFTLGYHGFDVWG |
| | | QGTLVTVSS |
| SEQ ID | Light chain | DIQMTQSPSSLSASVGDRVTITCRASQSINTYLNWYQ |
| NO: 18 | variable | QKPGKAPKLLIYDASNLQSGVPSRFSGSGSGTDFTLT |
| | | ISSLQPEDFATYYCQQYDDLPFTFGQGTKVEIK |

Anti-Mac-1 antibody sequence clone: 24G10
(Underline is a CDR sequence)

| | | |
|---|---|---|
| SEQ ID | Heavy chain | EVQLVESGGGLVQPGGSLRLSCAASGFTFSSNSMSWV |
| NO: 19 | variable | RQAPGKGLEWVSAINYSGREIYYADSVKGRFTISRDN |
| | | SKNTLYLQMNSLRAEDTAVYYCARTDYNTFDYWGQGT |
| | | LVTVSS |
| SEQ ID | Light chain | DIQMTQSPSSLSASVGDRVTITCRASQSNSSHLNWYQ |
| NO: 20 | variable | QKPGKAPKLLIYGVSNLQSGVPSRFSGSGSGTDFTLT |
| | | ISSLQPEDFATYYCQHYGSTPYTFGQGTKVEIK |

Anti-Mac-1 antibody sequence clone: 24G11
(Underline is a CDR sequence)

| | | |
|---|---|---|
| SEQ ID | Heavy chain | EVQLVESGGGLVQPGGSLRLSCAASGFTFSDYYMSWV |
| NO: 21 | variable | RQAPGKGLEWVSVISYGGGEAYYADSVKGRFTISRDN |
| | | SKNTLYLQMNSLRAEDTAVYYCARAMASEYGPWDYWG |
| | | QGTLVTVSS |
| SEQ ID | Light chain | DIQMTQSPSSLSASVGDRVTITCRASQSISRHLTWYQ |
| NO: 22 | variable | QKPGKAPKLLIYGASTLQSGVPSRFSGSGSGTDFTLT |
| | | ISSLQPEDFATYYCQQYHDWPLTFGQGTKVEIK |

Anti-Mac-1 antibody sequence clone: 24G12
(Underline is a CDR sequence)

| | | |
|---|---|---|
| SEQ ID | Heavy chain | EVQLVESGGGLVQPGGSLRLSCAASGFTFGDDYMHWV |
| NO: 23 | variable | RQAPGKGLEWVSAINYDGSWKYYADSVKGRFTISRDN |
| | | SKNTLYLQMNSLRAEDTAVYYCARLSSIDEPPYGPFD |
| | | VWGQGTLVTVSS |
| SEQ ID | Light chain | DIQMTQSPSSLSASVGDRVTITCRASQSISTYLAWYQ |
| NO: 24 | variable | QKPGKAPKLLIYEASSLQSGVPSRFSGSGSGTDFTLT |
| | | ISSLQPEDFATYYCQQYYNFPPTFGQGTKVEIK |

Anti-Mac-1 antibody sequence clone: 24H01
(Underline is a CDR sequence)

| | | |
|---|---|---|
| SEQ ID | Heavy chain | EVQLVESGGGLVQPGGSLRLSCAASGFTFNNYYMSWV |
| NO: 25 | variable | RQAPGKGLEWVSIIYYDGSEADYADSVKGRFTISRDN |
| | | SKNTLYLQMNSLRAEDTAVYYCARNKDIYSVYGMDYW |
| | | GQGTLVTVSS |
| SEQ ID | Light chain | DIQMTQSPSSLSASVGDRVTITCRASQSISNYLAWYQ |
| NO: 26 | variable | QKPGKAPKLLIYATSNLQSGVPSRFSGSGSGTDFTLT |
| | | ISSLQPEDFATYYCQQANNTPPTFGQGTKVEIK |

Anti-Mac-1 antibody sequence clone: 24H02
(Underline is a CDR sequence)

| | | |
|---|---|---|
| SEQ ID | Heavy chain | EVQLVESGGGLVQPGGSLRLSCAASGFTFSDYWMSWV |
| NO: 27 | variable | RQAPGKGLEWVSSIVYGGSEIDYADSVKGRFTISRDN |
| | | SKNTLYLQMNSLRAEDTAVYYCARVPGYSGTPFDYWG |
| | | QGTLVTVSS |
| SEQ ID | Light chain | DIQMTQSPSSLSASVGDRVTITCRASQSVSRYLAWYQ |
| NO: 28 | variable | QKPGKAPKLLIYDTSSLQSGVPSRFSGSGSGTDFTLT |
| | | ISSLQPEDFATYYCQQSYSFPLTFGQGTKVEIK |

Anti-Mac-1 antibody sequence clone: 24H03
(Underline is a CDR sequence)

| | | |
|---|---|---|
| SEQ ID | Heavy chain | EVQLVESGGGLVQPGGSLRLSCAASGFTFKDSWMHWV |
| NO: 29 | variable | RQAPGKGLEWVSIISYSGGEAIYADSVKGRFTISRDN |
| | | SKNTLYLQMNSLRAEDTAVYYCARDSGGSAMGFDIWG |
| | | QGTLVTVSS |
| SEQ ID | Light chain | DIQMTQSPSSLSASVGDRVTITCRASQSIHSYLNWYQ |
| NO: 30 | variable | QKPGKAPKLLIYGASSLQSGVPSRFSGSGSGTDFTLT |
| | | ISSLQPEDFATYYCQQYYRFPYTFGQGTKVEIK |

Anti-Mac-1 antibody sequence clone: 25A02
(Underline is a CDR sequence)

| | | |
|---|---|---|
| SEQ ID | Heavy chain | EVQLVESGGGLVQPGGSLRLSCAASGFTFSDWYLHWV |
| NO: 31 | variable | RQAPGKGLEWVSVINGGGSEIIYADSVKGRFTISRDN |
| | | SKNTLYLQMNSLRAEDTAVYYCARGGDGDGSGFDDWG |
| | | QGTLVTVSS |
| SEQ ID | Light chain | DIQMTQSPSSLSARVGDRVTITCRASQSIHSYLNWYQ |
| NO: 32 | variable | QKPGKAPKMLIYDASNLQSGVPSRFSGSGSGTDFTLT |
| | | ISSLQPEDFATYYCQQSGNYPFTFGQGTKVEIK |

Anti-Mac-1 antibody sequence clone: 25A04
(Underline is a CDR sequence)

| | | |
|---|---|---|
| SEQ ID | Heavy chain | EVQLVESGGGLVQPGGSLRLSCAASGFTFSFTAMHWV |
| NO: 33 | variable | RQAPGKGLEWVSAIIYNGSEADYADSVKGRFTISRDN |
| | | SKNTLYLQMNSLRAEDTAVYYCARANDYDHGCCDNYA |
| | | MDYWGQGTLVTVSS |
| SEQ ID | Light chain | DIQMTQSPSSLSASVGDRVTITCRASQGIGSYLYWYQ |
| NO: 34 | variable | QKPGKAPKLLIYDASNLQSGVPSRFSGSGSGTDFTLT |
| | | ISSLQPEDFATYYCMQHGGWPLTFGQGTKVEIK |

Anti-Mac-1 antibody sequence clone: 25A06
(Underline is a CDR sequence)

| | | |
|---|---|---|
| SEQ ID | Heavy chain | EVQLVESGGGLVQPGGSLRLSCAASGFTFSSYYMSWV |
| NO: 35 | variable | RQAPGKGLEWVSTINYDGSEKDYADSVKGRFTISRDN |
| | | SKNTLYLQMNSLRAEDTAVYYCARDRLGNYPWFDVWG |
| | | QGTLVTVSS |
| SEQ ID | Light chain | DIQMTQSPSSLSASVGDRVTITCRASQSISNYLAWYQ |
| NO: 36 | variable | QKPGKAPKLLIYDANNLQSGVPSRFSGSGSGTDFTLT |
| | | ISSLQPEDFATYYCQQSYSWPLTFGQGTKVEIK |

Anti-Mac-1 antibody sequence clone: 25A09
(Underline is a CDR sequence)

| | | |
|---|---|---|
| SEQ ID | Heavy chain | EVQLVESGGGLVQPGGSLRLSCAASGFTFTEWWMSWV |
| NO: 37 | variable | RQAPGKGLEWVSTISYGGSEAIYADSVKGRFTISRDN |
| | | SKNTLYLQMNSLRAEDTAVYYCARTSSDRLLFDYWGQ |
| | | GTLVTVSS |
| SEQ ID | Light chain | DIQMTQSPSSLSASVGDRVTITCRASQSIKSSLAWYQ |
| NO: 38 | variable | QKPGKAPKLLIYGASTLQSGVPSRFSGSGSGTDFTLT |
| | | ISSLQPEDFATYYCMQTNRHPWTFGQGTKVEIK |

Anti-Mac-1 antibody sequence clone: 25A10
(Underline is a CDR sequence)

| | | |
|---|---|---|
| SEQ ID | Heavy chain | EVQLVESGGGLVQPGGSLRLSCAASGFTFSNYNLHWV |
| NO: 39 | variable | RQAPGKGLEWVSTISYSGSEIIYADSVKGRFTISRDN |
| | | SKNTLYLQMNSLRAEDTAVYYCAREDEYTYYYFDPWG |
| | | QGTLVTVSS |
| SEQ ID | Light chain | DIQMTQSPSSLSASVGDRVTITCRASQSVSSYLAWYQ |
| NO: 40 | variable | QKPGKAPKLLIYDASKLQSGVPSRFSGSGSGTDFTLT |
| | | ISSLQPEDFATYYCKQSYSSPPTFGQGTKVEIK |

Anti-Mac-1 antibody sequence clone: 25B03
(Underline is a CDR sequence)

| | | |
|---|---|---|
| SEQ ID | Heavy chain | EVQLVESGGGLVQPGGSLRLSCAASGFTFSDYWMHWV |
| NO: 41 | variable | RQAPGKGLEWVSTIIGGGSEAGYADSVKGRFTISRDN |
| | | SKNTLYLQMNSLRAEDTAVYYCARDRSYGYLGFDIWG |
| | | QGTLVTVSS |
| SEQ ID | Light chain | DIQMTQSPSSLSASVGDRVTITCRASQSIRNSLHWYQ |
| NO: 42 | variable | QKPGKAPKLLIYSAGKLQSGVPSRFSGSGSGTDFTLT |
| | | ISSLQPEDFATYYCQQSNSFPLTFGQGTKVEIK |

Anti-Mac-1 antibody sequence clone: 25B04
(Underline is a CDR sequence)

| | | |
|---|---|---|
| SEQ ID | Heavy chain | EVQLVESGGGLVQPGGSLRLSCAASGFTFSTNWMHWV |
| NO: 43 | variable | RQAPGKGLEWVSMISYSGGEAIYADSVKGRFTISRDN |
| | | SKNTLYLQMNSLRAEDTAVYYCARNWLPYAMDYWGQG |
| | | TLVTVSS |
| SEQ ID | Light chain | DIQMTQSPGSLSASVGDRVTITCRASQSIRSYLSWYQ |
| NO: 44 | variable | QKPGKAPKLLIYSASTLQSGVPSRFSGSGSGTDFTLT |
| | | ISSLQPEDFATYYCQQSYSTPLTFGQGTKVEIK |

Anti-Mac-1 antibody sequence clone: 25B01
(Underline is a CDR sequence)

| | | |
|---|---|---|
| SEQ ID | Heavy chain | AVQLVESGGGLVQPGGSLRLSCAASGFTFSSYDVGWV |
| NO: 45 | variable | RQAPGKGLEWVSGIVPSGGNIYYADSVKGRFTISRDN |
| | | SKNTLYLQMNSLRAEDTAVYYCARHRSYAYYAFDYWG |
| | | QGTLVTVSS |
| SEQ ID | Light chain | DIQMTQSPSSLSASVGDRVTITCRASQNVRNYLGWYQ |
| NO: 46 | variable | QKPGKAPKLLIYDASSLQSGVPSRFSGSGSGTDFTLT |
| | | ISSLQPEDFATYYCQQYGDWPLTFGQGTKVEIK |

Anti-Mac-1 antibody sequence clone: DF3-10
(Underline is a CDR sequence)

| | | |
|---|---|---|
| SEQ ID | Heavy chain | EVQLVESGGGLVQPGGSLRLSCAASGFTFTDAYMSWV |
| NO: 47 | variable | RQAPGKGLEWVSTISSYGSSTYYADSVKGRFTISRDN |
| | | SKNTLYLQMNSLRAEDTAVYYCARPRYIESPVYDYWG |
| | | QGTLVTVSS |
| SEQ ID | Light chain | DIQMTQSPSSLSASVGDRVTITCRASQSIAKYLAWYQ |
| NO: 48 | variable | QKPGKAPKLLIYETSNLQSGVPSRFSGSGSGTDFTLT |
| | | ISSLQPEDFATYYCQQSSSSPETFGQGTKVEIK |

Anti-Mac-1 antibody sequence clone: DF3-28
(Underline is a CDR sequence)

| | | |
|---|---|---|
| SEQ ID | Heavy chain | EVQLVESGGGLVQPGGSLRLSCAASGFTFTNYWMHWV |
| NO: 49 | variable | RQAPGKGLEWVSTIIYDGGETGYADSVKGRFTISRDN |
| | | SKNTLYLQMNSLRAEDTAVYYCARNSRKSGMDYWGQG |
| | | TLVTVSS |
| SEQ ID | Light chain | DIQMTQSPSSLSASVGDRVTITCRASQSIYKYLNWYQ |
| NO: 50 | variable | QKPGKAPKLLIYDASTLQSGVPSRFSGSGSGTDFTLT |
| | | ISSLQPEDFATYYCMQYYSDPLTFGQGTKVEIK |

Anti-Mac-1 antibody sequence clone: DF3-30
(Underline is a CDR sequence)

| | | |
|---|---|---|
| SEQ ID | Heavy chain | EVQLVESGGGLVQPGGSLRLSCAASGFTFSGYAWSWV |
| NO: 51 | variable | RQAPGKGLEWVSMISPAGGSTYYADSVKGRFTISRDN |
| | | SKNTLYLQMNSLRAEDTAVYYCARDRNAGGDSYYSFD |
| | | VWGQGTLVTVSS |
| SEQ ID | Light chain | DIQMTQSPSSLSASVGDRVTITCRASQSINSHLAWYQ |
| NO: 52 | variable | QKPGKAPKLLIYAAINLQSGVPSRFSGSGSGTDFTLT |
| | | ISSLQPEDFATYYCQQTNHYPLTFGQGTKVEIK |

Anti-Mac-1 antibody sequence clone: DF3-32
(Underline is a CDR sequence)

| | | |
|---|---|---|
| SEQ ID | Heavy chain | EVQLVESGGGLVQPGGSLRLSCAASGFTFSSHAMHWV |
| NO: 53 | variable | RQAPGKGLEWVSSILSGGSETNYADSVKGRFTISRDN |
| | | SKNTLYLQMNSLRAEDTAVYYCARDTYEVTGNLLDYW |
| | | GQGTLVTVSS |
| SEQ ID | Light chain | DIQMTQSPSSLSASVGDRVTITCRASQSVWSYLNWYQ |
| NO: 54 | variable | QKPGKAPKLLIYGASSLQSGVPSRFSGSGSGTDFTLT |
| | | ISSLQPEDFATYYCMQSYSWPFTFGQGTKVEIK |

Anti-Mac-1 antibody sequence clone: DF4-16
(Underline is a CDR sequence)

| | | |
|---|---|---|
| SEQ ID | Heavy chain | EVQLVESGGGLVQPGGSLRLSCAASGFTFNEYAMSWV |
| NO: 55 | variable | RQAPGKGLEWVSSIIPDGSETDYADSVKGRFTISRDN |
| | | SKNTLYLQMNSLRAEDTAVYYCARSLSSSGMHGDIWG |
| | | QGTLVTVSS |
| SEQ ID | Light chain | DIQMTQSPSSLSASVGDRVTITCRASQSINNYLNWYQ |
| NO: 56 | variable | QKPGKAPKLLIYKASTLQSGVPSRFSGSGSGTDFTLT |
| | | ISSLQPEDFATYYCHQYHSPYTFGQGTKVEIK |

Anti-Mac-1 antibody sequence clone: DF4-17
(Underline is a CDR sequence)

| | | |
|---|---|---|
| SEQ ID | Heavy chain | EVQLVESGGGLVQPGGSLRLSCAASGFTFSDYGWHWV |
| NO: 57 | variable | RQAPGKGLEWVSIIESDGSGTYYADSVKGRFTISRDN |
| | | SKNTLYLQMNSLRAEDTAVYYCARNGEVGERGVRDYD |
| | | YAMDYWGQGTLVTVSS |
| SEQ ID | Light chain | DIQMTQSPRSLSASVGDRVTITCRASQSINRYLNWYQ |
| NO: 58 | variable | QKPGKAPKLLIYATSSLQSGVPSRFSGSGSGTDFTLT |
| | | ISSLQPEDFATYYCQQYGSTPITFGQGTKVEIK |

Anti-Mac-1 antibody sequence clone: DF4-22
(Underline is a CDR sequence)

| | | |
|---|---|---|
| SEQ ID | Heavy chain | EVQLVESGGGLVQPGGSLRLSCAASGFTFTDYNVHWV |
| NO: 59 | variable | RQAPGKGLEWVSGINSSGSETNYADSVKGRFTISRDN |
| | | SKNTLYLQMNSLRAEDTAVYYCARDSVFKTVGGYDAV |
| | | MDYWGQGTLVTVSS |
| SEQ ID | Light chain | DIQMTQSPSSLSASVGDRVTITCRASQSIYNYLAWYQ |
| NO: 60 | variable | QKPGKAPKLLIYGTSTLQSGVPSRFSGSGSGTDFTLT |
| | | ISSLQPEDFATYYCMQSYSSPTTFGQGTKVEIK |

Anti-Mac-1 antibody sequence clone: DF4-25
(Underline is a CDR sequence)

| | | |
|---|---|---|
| SEQ ID | Heavy chain | EVQLVESGGGLVQPGGSLRLSCAASGFTFKDYWLSWV |
| NO: 61 | variable | RQAPGKGLEWVSIINYGGSETYYADSVKGRFTISRDN |
| | | SKNTLYLQMNSLRAEDTAVYYCARTQTSYVMDYWGQG |
| | | TLVTVSS |
| SEQ ID | Light chain | DIQMTQSPSSLSASVGDRVTITCRASQSVRSGLNWYQ |
| NO: 62 | variable | QKPGKAPKLLIYAASSLQSGVPRRFSGSGSGTDFTLT |
| | | ISSLQPEDFATYYCMQSHSWPLTFGQGTKVEIK |

Anti-Mac-1 antibody sequence clone: DF4-26
(Underline is a CDR sequence)

| | | |
|---|---|---|
| SEQ ID | Heavy chain | EVQLVESGGGLVQPGGSLRLSCAASGFTFSSGYMSWV |
| NO: 63 | variable | RQAPGKGLEWVSTISGSGRETNYADSVKGRFTISRDN |
| | | SKNTLYLQMNSLRAEDTAVYYCARDAWGGDSYFDPWG |
| | | QGTLVTVSS |
| SEQ ID | Light chain | DIQMTQSPGSLSASVGDRVTITCRASQSIWSNLSWYQ |
| NO: 64 | variable | QKPGKAPKLLIYNASSLQSGVPSRFSGSGSGTDFTLT |
| | | ISSLQPEDFATYYCQQYHGTPITFGQGTKVEIK |

Anti-Mac-1 antibody sequence clone: DF4-42
(Underline is a CDR sequence)

| | | |
|---|---|---|
| SEQ ID | Heavy chain | EVQLVESGGGLVQPGGSLRLSCAASGFTFTDYQMSWV |
| NO: 65 | variable | RQAPGKGLEWVSTIIWSGSETNYADSVKGRFTISRDN |
| | | SKNTLYLQMNSLRAEDTAVYYCARNKTPFDYWGQGTL |
| | | VTVSS |
| SEQ ID | Light chain | DIQMTQSPRSLSASVGDRVTITCRASQSIRTHLAWYQ |
| NO: 66 | variable | QKPGKAPKLLIYDNSNLQSGVPSRFSGSGSGTDFTLT |
| | | ISSLQPEDFATYYCQQYKGSPLTFGQGTKVEIK |

Anti-Mac-1 antibody sequence clone: DF3M-1
(Underline is a CDR sequence)

| | | |
|---|---|---|
| SEQ ID | Heavy chain | EVQLVESGGGLVQPGGSLRLSCAASGFTFTSYAMSWV |
| NO: 67 | variable | RQAPGKGLEWVSSISYSGGETDYADSVKGRFTISRDN |
| | | SKNTLYLQMNSLRAEDTAVYYCARSKGGLYFDYWGQG |
| | | TLVTVSS |
| SEQ ID | Light chain | DIQMTQSPGSLSASVGDRVTITCRASQSISSYLAWYQ |
| NO: 68 | variable | QKPGKAPKLLIYGASSLQSGVPSRFSGSGSGTDFTLT |
| | | ISSLQPEDFATYYCQQYGSTPETFGQGTKVEIK |

Anti-Mac-1 antibody sequence clone: DF3M-2
(Underline is a CDR sequence)

| | | |
|---|---|---|
| SEQ ID | Heavy chain | EVQLVESGGGLVQPGGSLRLSCAASGFTFSGYWIHWV |
| NO: 69 | variable | RQAPGKGLEWVSTISYSGDEAYYADSVKGRFTISRDN |
| | | SKNTLYLQMNSLRAEDTAVYYCARSPSDGDYGFDYWG |
| | | QGTLVTVSS |
| SEQ ID | Light chain | DIQMTQSPSSLSASVGDRVNITCRASQSINNYLSWYQ |
| NO: 70 | variable | QKPGKAPKLLIYDGRILQSGVPSRFSGSGSGTDFTLT |
| | | ISSLQPEDFATYYCMQYLAYPWTFGQGTKVEIK |

Anti-Mac-1 antibody sequence clone: DF3M-5
(Underline is a CDR sequence)

| | | |
|---|---|---|
| SEQ ID | Heavy chain | EVQLVESGGGLVQPGGSLRLSCAASGFTFGTYDMHWV |
| NO: 71 | variable | RQAPGKGLEWVSMISPSGGDTYYADSVKGRFTISRDN |
| | | SKNTLYLQMNSLRAEDTAVYYCARDSSGDWYAMAYWG |
| | | QGTLVTVSS |
| SEQ ID | Light chain | DIQMTQSPSSLSASVGDRVTITCRASQSIRRYLAWYQ |
| NO: 72 | variable | QKPGKAPKLLIYGASNLQSGVPSRFSGSGSGTDFTLT |
| | | ISSLQPEDFATYYCQHSSDTPLTFGQGTKVEIK |

Anti-Mac-1 antibody sequence clone: DF3M-18
(Underline is a CDR sequence)

| | | |
|---|---|---|
| SEQ ID | Heavy chain | EVQLVESGGGLVQPGGSLRLSCAASGFTFSDSAMGWV |
| NO: 73 | variable | RQAPGKGLEWVSIISYYGSETYYADSVKGRFTISRDN |
| | | SKNTLYLQMNSLRAEDTAVYYCARNPDGDLSALDYWG |
| | | QGTLVTVSS |
| SEQ ID | Light chain | DIQMTQSPSSLSASVGDRVTITCRASQPISSYLNWYQ |
| NO: 74 | variable | QKPGKAPKLLIYGASSLQSGVPSRFSGSGSGTDFTLT |
| | | ISSLQPEDFATYYCQQRLRSPFTFGQGTKVEIK |

Anti-Mac-1 antibody sequence clone: DF3M-19
(Underline is a CDR sequence)

| | | |
|---|---|---|
| SEQ ID | Heavy chain | EVQLVESGGGLVQPGGSLRLSCAASGFTFTSYAMSWV |
| NO: 75 | variable | RQAPGKGLEWVSSINSGGSETNYADSVKGRFTISRDN |
| | | SKNTLYLQMNSLRAEDTAVYYCARGEYYTDVWPSGFD |
| | | IWGQGTLVTVSS |
| SEQ ID | Light chain | DIQMTQSPSSLSASVGDRVTITCRASQSISNPLNWYQ |
| NO: 76 | variable | QKPGKAPKLLIYDASTLQSGVPSRFSGSGSGTDFTLT |
| | | ISSLQPEDFATYYCQQYGSSPSTFGQGTKVEIK |

Anti-Mac-1 antibody sequence clone: DF3M-30
(Underline is a CDR sequence)

| | | |
|---|---|---|
| SEQ ID | Heavy chain | EVQLVESGGGLVQPGGSLRLSCAASGFTFSNYEMGWV |
| NO: 77 | variable | RQAPGKGLEWVSIISWSGSETIYADSVKGRFTISRDN |
| | | SKNTLYLQMNSLRAEDTAVYYCARNGRGDYAFDFWGQ |
| | | GTLVTVSS |
| SEQ ID | Light chain | DIQMTQSPSSLSASVGDRVTITCRASQSVSNNLAWYQ |
| NO: 78 | variable | QKPGKAPKLLIYRTTSLQSGVPSRFSGSGSGTDFTLT |
| | | ISSLQPEDFATYYCMQYGSLPSTFGQGTKVEIK |

Anti-Mac-1 antibody sequence clone: DF3M-36
(Underline is a CDR sequence)

| | | |
|---|---|---|
| SEQ ID | Heavy chain | EVQLVESGGGLVQPGGSLRLSCAASGFTFSDYGMSWV |
| NO: 79 | variable | RQAPGKGLEWVSIISPGGRETYYADSVKGRFTISRDN |
| | | SKNTLYLQMNSLRAEDTAVYYCARSPDGGYYEFDVWG |
| | | QGTLVTVSS |
| SEQ ID | Light chain | DIQMTQSPSSLSASVGDRVTITCRASQSISNYLAWYQ |
| NO: 80 | variable | QKPGKAPKLLIYDASTLQSGVPSRFSGSGSGTDFTLT |
| | | ISSLQPEDFATYYCHQRNSWPPTFGQGTKVEIK |

Anti-Mac-1 antibody sequence clone: DF3M-42
(Underline is a CDR sequence)

| | | |
|---|---|---|
| SEQ ID | Heavy chain | EVQLVESGGGLVQPGGSLRLSCAASGFTFSSYHMHWV |
| NO: 81 | variable | RQAPGKGLEWVSAIDSSGRETFYADSVKGRFTISRDN |
| | | SKNTLYLQMNSLRAEDTAVYYCARGYGDYFDYWGQGT |
| | | LVTVSS |
| SEQ ID | Light chain | DIQMTQSPGSLSASVGDRVTITCRASQSGSNYLAWYQ |
| NO: 82 | variable | QKPGKAPKLLIYAASTLQSGVPSRFSGSGSGTDFTLT |
| | | ISSLQPEDFATYYCQQSGSTPYTFGQGTKVEIK |

Anti-Mac-1 antibody sequence clone: DF4M-1
(Underline is a CDR sequence)

| | | |
|---|---|---|
| SEQ ID | Heavy chain | EVQLVESGGGLVQPGGSLRLSCAASGFTFDNYVMGWV |
| NO: 83 | variable | RQAPGKGLEWVSMINYGGSETIYADSVKGRFTISRDN |
| | | SKNTLYLQMNSLRAEDTAVYYCARSACDYCDFDYWGQ |
| | | GTLVTVSS |
| SEQ ID | Light chain | DIQMTQSPSSLSASVGDRVTITCRASQVVGSYLNWYQ |
| NO: 84 | variable | QKPGKAPKLLIYGASTLQSGVPSRFSGSGSGTDFTLT |
| | | ISSLQPEDFATYYCQQYYNYPGTFGQGTKVEIK |

Anti-Mac-1 antibody sequence clone: DF4M-3
(Underline is a CDR sequence)

| | | |
|---|---|---|
| SEQ ID | Heavy chain | EVQLVESGGGLVQPGGSLRLSCAASGFTFTTYYMSWV |
| NO: 85 | variable | RQAPGKGLEWVSTIIPSGSETNYADSVKGRFTISRDN |
| | | SKNTLYLQMNSLRAEDTAVYYCARVPAASEGPMDYWG |
| | | QGTLVTVSS |
| SEQ ID | Light chain | DIQMTQSPSSLSASVGDRVTITCRASQSISRNLAWYQ |
| NO: 86 | variable | QKPGKAPKLLIYDASSLQSGVPSRFSGSGSGTDFTLT |
| | | ISSLQPEDFATYYCQQYYHSPPTFGQGTKVEIK |

Anti-Mac-1 antibody sequence clone: DF4M-7-1
(Underline is a CDR sequence)

| | | |
|---|---|---|
| SEQ ID | Heavy chain | EVQLVESGGGLVQPGGSLRLSCAASGFTFDSYAMHWV |
| NO: 87 | variable | RQAPGKGLEWVSSIDGSGRETDYADSVKGRFTISRDN |
| | | SKNTLYLQMNSLRAEDTAVYYCARDGSEGYAFDPWGQ |
| | | GTLVTVSS |
| SEQ ID | Light chain | DIQMTQSPSSLSASVGDRVTITCRASQIIRHKLNWYQ |
| NO: 88 | variable | QKPGKAPKLLIYDASTLQSGVPSRFSGSGSGTDFTLT |
| | | ISSLQPEDFATYYCQQYNSWPITFGQGAKVEIK |

Anti-Mac-1 antibody sequence clone: DF4M-7-4
(Underline is a CDR sequence)

| | | |
|---|---|---|
| SEQ ID | Heavy chain | EVQLVESGGGLVQPGGSLRLSCAASGFTFTSYIMSWV |
| NO: 89 | variable | RQAPGKGLEWVSIISYSGGETYYADSVKGRFTISRDN |
| | | SKNTLYLQMNSLRAEDTAVYYCARNGINDDSFDYWGQ |
| | | GTLVTVSS |
| SEQ ID | Light chain | DIQMTQSPSSLSASVGDRVTITCRASQSISNYLNWYQ |
| NO: 90 | variable | QKPGKAPKLLIYGASSLQSGVPSRFSGSGSGTDFTLT |
| | | ISSLQPEDFATYYCQQRLHWPGTFGQGTKVEIK |

Anti-Mac-1 antibody sequence clone: DF4M-9
(Underline is a CDR sequence)

| | | |
|---|---|---|
| SEQ ID | Heavy chain | EVQLVESGGGLVQPGGSLRLSCAASGFTFTNHWMSWV |
| NO: 91 | variable | RQAPGKGLEWVSTIEGSGSETIYADSVKGRFTISRDN |
| | | SKNTLYLQMNSLRAEDTAVYYCARSSRTLFDYWGQGT |
| | | LVTVSS |
| SEQ ID | Light chain | DIQMTQSPSSLSASVGDRVTITCRASQGVYSYLAWYQ |
| NO: 92 | variable | QKPGKAPKLLIYDASSLQSGVPSRFSGSGSGTDFTLT |
| | | ISSLQPEDFATYYCQQYYHYPPTFGQGTKVEIK |

Anti-Mac-1 antibody sequence clone: DF4M-11
(Underline is a CDR sequence)

| | | |
|---|---|---|
| SEQ ID | Heavy chain | EVQLVESGGGLVQPGGSLRLSCAASGFTFSNYYMDWV |
| NO: 93 | variable | RQAPGKGLEWVSSINPWGGNKYYADSVKGRFTISRDN |
| | | SKNTLYLQMNSLRAEDTAVYYCARTITSKYEDYAMDY |
| | | WGQGTLVTVSS |
| SEQ ID | Light chain | DIQMTQSPSSLSASVGDRVTITCRASQSISSYLAWYQ |
| NO: 94 | variable | QKPGKAPKLLIYLTSNLQSGVPSRFSGSGSGTDFTLT |
| | | ISSLQPEDFATYYCQQTAQNPFTFGQGTKVEIK |

Anti-Mac-1 antibody sequence clone: DF4M-17
(Underline is a CDR sequence)

| | | |
|---|---|---|
| SEQ ID | Heavy chain | EVQLVESGGGLVQPGGSLRLSCAASGFTFSDYWVAWV |
| NO: 95 | variable | RQAPGKGLEWVSTISYSGSETEYADSVKGRFTISRDN |
| | | SKNTLYLQMNSLRAEDTAVYYCARYGGSDYYGFDPWG |
| | | QGTLVTVSS |
| SEQ ID | Light chain | DIQMTQSPSSLSASVGDRVTITCRASQSISNNLAWYQ |
| NO: 96 | variable | QKPGKAPKLLIYATTTLQSGVPSRFSGSGSGTDFTLT |
| | | ISSLQPEDFATYYCMQSNTPWTFGQGTKVEIK |

Anti-Mac-1 antibody sequence clone: DF4M-18
(Underline is a CDR sequence)

| | | |
|---|---|---|
| SEQ ID | Heavy chain | EVQLVESGGGLVQPGGSLRLSCAASGFTFSSYAISWV |
| NO: 97 | variable | RQAPGKGLEWVSAISSGGSETDYADSVKGRFTISRDN |
| | | SKNTLYLQMNSLRAEDTAVYYCARGESGYYMAEDVWG |
| | | QGTLVTVSS |
| SEQ ID | Light chain | DIQMTQSPSSLSASVGDRVTITCRASQSVSSFLAWYQ |
| NO: 98 | variable | QKPGKAPKLLIYAASKLQSGVPSRFSGSGSGTDFTLT |
| | | ISSLQPEDFATYYCQQYSVTPITFGQGTKVEIK |

Anti-Mac-1 antibody sequence clone: DF4M-21
(Underline is a CDR sequence)

| | | |
|---|---|---|
| SEQ ID | Heavy chain | EVQLVESGGGLVQPGGSLRLSCAASGFTFSSYAMSWV |
| NO: 99 | variable | RQAPGKGLEWVSAISSYGGETDYADSVKGRFTISRDN |
| | | SKNTLYLQMNSLRAEDTAVYYCARGDAYSSFVDNPFD |
| | | IWGQGTLVTVSS |
| SEQ ID | Light chain | DIQMTQSPSSLSASVGDRVTITCRASQSISNYLAWYQ |
| NO: 100 | variable | QKPGKAPKLLIYDASTLQSGVPSRFSGSGSGTDFTLT |
| | | ISSLQPEDFATYYCMQYESTPWTFGQGTKVEIK |

Anti-Mac-1 antibody sequence clone: DF4M-23
(Underline is a CDR sequence)

| | | |
|---|---|---|
| SEQ ID | Heavy chain | EVQLVESGGGLVQPGGSLRLSCAASGFTFTSYAMSWV |
| NO: 101 | variable | RQAPGKGLEWVSAISPSGSETEYADSVKGRFTISRDN |
| | | SKNTLYLQMNSLRAEDTAVYYCARGFYNDYIFDLWGQ |
| | | GTLVTVSS |
| SEQ ID | Light chain | DIQMTQSPSSLSASVGDRVTITCRASQSISSYLNWYQ |
| NO: 102 | variable | QKPGKAPKLLIYAASSLQSGVPSRFSGSGSGTDFTLT |
| | | ISSLQPEDFATYYCQQYLSTPYTFGQGTKVEIK |

Anti-Mac-1 antibody sequence clone: DF4M-30
(Underline is a CDR sequence)

| | | |
|---|---|---|
| SEQ ID | Heavy chain | EVQLVESGGGLVQPGGSLRLSCAASGFTFRNNAMHWV |
| NO: 103 | variable | RQAPGKGLEWVSVINSGGSETYYADSVKGRFTISRDN |
| | | SKNTLYLQMNSLRAEDTAVYYCARDEPSDEYGMYGFD |
| | | YWGQGTLVTVSS |
| SEQ ID | Light chain | DIQMTQSPSSLSASVGDRVTITCRASQSISSYLNWYQ |
| NO: 104 | variable | QKPGKAPKLLIYKASNLQSGVPSRFSGSGSGTDFTLT |
| | | ISSLQPEDFATYYCVQYSRSPTTFGQGTKVEIK |

Anti-Mac-1 antibody sequence clone: DF4M-31
(Underline is a CDR sequence)

| | | |
|---|---|---|
| SEQ ID | Heavy chain | EVQLVESGGGLVQPGGSLRLSCAASGFTFTSATMSWV |
| NO: 105 | variable | RQAPGKGLEWVSIISPGGSETYYADSVKGRFTISRDN |
| | | SKNTLYLQMNSLRAEDTAVYYCARGGDYPSYYMDPWG |
| | | QGTLVTVSS |
| SEQ ID | Light chain | DIQMTQSPSSLSASVGDRVTITCRASQSISNYLAWYQ |
| NO: 106 | variable | QKPGKAPKLLIYGTSSLQSGVPSRFSGSGSGTDFTLT |
| | | ISSLQPEDFATYYCQQGHQWPWTFGQGTKVEIK |

Anti-Mac-1 antibody sequence clone: DF4M-45
(Underline is a CDR sequence)

| | | |
|---|---|---|
| SEQ ID | Heavy chain | EVQLVESGGGLVQPGGSLRLSCAASGFTFTSYYMSWV |
| NO: 107 | variable | RQAPGKGLEWVSTIISDGSETGYADSVKGRFTISRDN |
| | | SKNTLYLQMNSLRAEDTAVYYCARTNRYGFQFDYWGQ |
| | | GTLVTVSS |
| SEQ ID | Light chain | DIQMTQSPSSLSASVGDRVTITCRASQGARNGLHWYQ |
| NO: 108 | variable | QKPGKAPKLLIYDASTLQSGVPSRFSGSGSGTDFTLT |
| | | ISSLQPEDFATYYCQQRYSYPPTFGQGTKVEIK |

Anti-Mac-1 antibody sequence clone: 28E07
(Underline is a CDR sequence)

| | | |
|---|---|---|
| SEQ ID | Heavy chain | QVQLQQSGPELVKPGASVKISCKASGYSFTDYNMNWV |
| NO: 109 | variable | KQSNGKSLEWIGEINPGYGTSRYNQKFKDKATLTVDQ |
| | | SSTTAYMQLNSLTSEDSAVYYCARADVDYGDVMDYWG |
| | | QGTTVTVSS |
| SEQ ID | Light chain | DIELTQSPAIMSASPGEKVTMTCSASSSVSYMHWYQQ |
| NO: 110 | variable | KSGTSPKRWIYDTSKLASGVPTRFSGSGSGTSYSLTI |
| | | SSMEAEDAATYYCQQWSSNPPTFGAGTKLELK |

Anti-Mac-1 antibody sequence clone: 28A12
(Underline is a CDR sequence)

| | | |
|---|---|---|
| SEQ ID | Heavy chain | QVKLQESGPELVKPGASVKISCKASGYSFTDYNMNWV |
| NO: 111 | variable | KQSNGKSLEWIGEINPGYGTSRYNQKFKDKATLTVDQ |
| | | SSSTAYMQLNSLTSEDSAVYYCARADVDYGDTMDYWG |
| | | QGTTVTVSS |
| SEQ ID | Light chain | DIELTQSPAIMSASPGEKVTMTCSASSSVSDMHWYQQ |
| NO: 112 | variable | KSGNSPKRWIYDTSKLASGVPVRFSGSGSGTSYSLTI |
| | | SSMEAEDAATYYCQQWSSNPPTFGAGTKLELK |

Anti-Mac-1 antibody sequence clone: 27G04
(Underline is a CDR sequence)

| | | |
|---|---|---|
| SEQ ID | Heavy chain | QVKLQESGPELVKPGASVKISCKASGYSFTDYNMNWV |
| NO: 113 | variable | KQSNGKSLEWIGVINPNYGTTSYNQKFKGKATLTVDQ |
| | | SSSTAYMQLNSLTSEDSAVYYCARTFDYDDDAFAYWG |
| | | QGTTVTVSS |
| SEQ ID | Light chain | DIELTQSPAIMSASPGEKVTITCSASSSVSDMHWYQQ |
| NO: 114 | variable | KSGTSPKRWIYDTSKLASGVPARFSGSGSGTSYSLTI |
| | | SNMEAEDAATYYCQQWSSNPPTFGAGTKLELK |

Anti-Mac-1 antibody sequence clone: 27A04
(Underline is a CDR sequence)

| | | |
|---|---|---|
| SEQ ID | Heavy chain | QVKLQQSGPELVKPGASVKISCKASGYSFTDYNMNWV |
| NO: 115 | variable | KQSNGKSLEWIGVINPNYGTTSYNQKFKGKATLTVDQ |
| | | SSSTAYMQLNSLTSEDSAVYYCARTYDYDGDAFAYWG |
| | | QGTTVTVSS |
| SEQ ID | Light chain | DIELTQSPAIMSASPGEKVTMTCSASSSVSYMHWYQQ |
| NO: 116 | variable | KSGTSPKRWIYDTSKLASGVPARFSGSGSGTSYSLTI |
| | | SSMEAEDAATYYCQQWSSNPPTFGAGTKLELK |

Anti-Mac-1 antibody sequence clone: 27A06
(Underline is a CDR sequence)

| | | |
|---|---|---|
| SEQ ID | Heavy chain | QVKLQQSGPELVTPGASVKISCKPSGYSFTDYNMNWV |
| NO: 117 | variable | KQSNGKSLEWIGEINPNYGTTRHNQKFKGKASLTVDQ |
| | | SSSTAYMQLISLTSEDSAVYYCARTYDYDEDAFAYWG |
| | | QGTTVTVSS |
| SEQ ID | Light chain | DIELTQSPAIMSASPGEKVTMTCSASSSVSYMHWYQQ |
| NO: 118 | variable | KSDTSPKRWIYDTSKLASGVPARFSGSGSGTSYSLTI |
| | | SSMEAEDAATYYCQQWSSNPPTFGAGTKLELK |

Anti-Mac-1 antibody sequence clone: 28G06
(Underline is a CDR sequence)

| | | |
|---|---|---|
| SEQ ID | Heavy chain | QVKLQQSGPELVKPGASVKISCKASGYSFTDYNMNWV |
| NO: 119 | variable | KQSNGKSLEWIGIINPNYGTTSYNQKFKGKATLTVDQ |
| | | SSSTAYMQLNSLTSEDSAVYYCARGYDYDESGFAYWG |
| | | QGTTVTVSS |
| SEQ ID | Light chain | DIELTQSPAIMSASPGEKVTMTCSASSSVSYMYWYQQ |
| NO: 120 | variable | KPGSSPRLLIYDTSNLASGVPVRFSGSGSGTSYSLTI |
| | | SRMEAEDAATYYCQQWSSNPPTFGGGTKLEIK |

Anti-Mac-1 antibody sequence clone: 27B10
(Underline is a CDR sequence)

| | | |
|---|---|---|
| SEQ ID | Heavy chain | QVQLQQSGPELVKPGASVKISCKTSGYSFTDYNMNWV |
| NO: 121 | variable | KQSNGKSLEWIGRINPNFGTTTYNQKFKGKATLTVDQ |
| | | SSSTAYMQLNSLTSEDSAVYYCARGYDYDESGFAYWG |
| | | QGTTVTVSS |
| SEQ ID | Light chain | DIELTQSPAIMSASPGEKVTMTCSASSSVSYMYWYQQ |
| NO: 122 | variable | KPGSSPRLLIYDTSNLASGVPVRFSGSGSGTSYSLTI |
| | | SRMEAEDAATYYCQQWSSYPPTFGGGTKLEIK |

Anti-Mac-1 antibody sequence clone: 27D06
(Underline is a CDR sequence)

| | | |
|---|---|---|
| SEQ ID | Heavy chain | QVKLQESGAEVVKPGASVKISCKASGYSFTDYNMNWV |
| NO: 123 | variable | KQSNGKSLEWIGVINPNYGTTSYNQKFKGKATLTVDQ |
| | | SSSTAYMQLNSLTSEDSAVYYCARTYDYDGDAFAYWG |
| | | QGTTVTVSS |
| SEQ ID | Light chain | DIELTQSPALMSASPGEKVTMTCRASSSVSSNNLHWY |
| NO: 124 | variable | QQKSGASPKLWIYSTSNLATGAPARFSGSGSGTSYSL |
| | | TISSMEAEDAATYYCQQWNSNPPTFGGGTKLEIK |

Anti-Mac-1 antibody sequence clone: 28D06
(Underline is a CDR sequence)

| | | |
|---|---|---|
| SEQ ID | Heavy chain | QVKLQQSGPELVKPGASVKISCKASGYSFTDYNMNWV |
| NO: 125 | variable | KQSNGKSLEWIGEINPNYGTTRYNQKFKGKATLTVDQ |
| | | SSSTAYMQLNSLTSEDSAVYYCARPSIYYDYDDAMDY |
| | | WGQGTTVTVSS |
| SEQ ID | Light chain | DIELTQSPAIMSASPGEKVTMTCSASSSVNYMYWYQQ |
| NO: 126 | variable | KPGSSPRLLIYDTSNLASGVPVRFSGSGSGTSYSLTI |
| | | SRMEAEDAATYYCQQWITYPPTLTFGAGTKLEIK |

Anti-Mac-1 antibody sequence clone: 27E12
(Underline is a CDR sequence)

| | | |
|---|---|---|
| SEQ ID | Heavy chain | QVQLQQSGTVLARPGASVKMSCKASGYTFTSYWMHWV |
| NO: 127 | variable | KQRPGQGLEWIGAIYPGNSDTSYNQKFKGKAKLTAVT |
| | | SASTAYMELSSLTNEDSAVYYCTRGGGSYEFAYWGQG |
| | | TTVTVSS |
| SEQ ID | Light chain | DIELTQSPAIMSASPGEKVTMTCSVSSSVSYMYWYQQ |
| NO: 128 | variable | KPGSSPRLLIYDTSNLASGVPARFSGSGSGTSYSLTI |
| | | SSMEAEDAATYYCQQWSSNPFTFGSGTKLEIK |

Anti-Mac-1 antibody sequence clone: m2396
(Underline is a CDR sequence)

| | | |
|---|---|---|
| SEQ ID | Heavy chain | QVQLQESGPGLVKPSETLSLTCTVSGFSLTSNSISWV |
| NO: 129 | variable | RQPPGKGLEWMGAIWSGGGTDYNPSLKSRLTISRDTS |
| | | KSQVFLKMSSLTAADTAIYFCTRGGYPYYFDYWGQGV |
| | | LVTVSS |
| SEQ ID | Light chain | DIVMTQSPDSLAVSLGERVTINCKSSQSLLYSENQEN |
| NO: 130 | variable | YLAWYQQKPGQSPKLLIYWASTRQSGVPDRFSGSGSG |
| | | TDFTLTISSVQAEDLAIYYCQQYYDTPLTFGGGTKLE |
| | | IK |

Anti-Mac-1 antibody sequence clone: H4L2
(Underline is a CDR sequence)

| | | |
|---|---|---|
| SEQ ID | Heavy chain | QVQLVQSGAEVKKPGASVKVSCKASGYTFTNYWINWV |
| NO: 131 | variable | RQAPGQRLEWMGNIYPSDTYINHNQKFKDRVTITRDT |
| | | SASTAYMELSSLRSEDTAVYYCARSAYANYFDYWGQG |
| | | TLVTVSS |
| SEQ ID | Light chain | EIVMTQSPATLSVSPGERATLSCRASQNIGTSIHWYQ |
| NO: 132 | variable | QKPGQAPRLLIYYASESISGIPARFSGSGSGTEFTLT |
| | | ISSLQSEDFAVYYCQQSDSWPTLTFGQGTKLEIK |

Anti-Mac-1 antibody sequence clone: 28E07-HH
(Underline is a CDR sequence)

| | | |
|---|---|---|
| SEQ ID | Heavy chain | QVQLVQSGAEVKKPGASVKVSCKASGYSFTDYNMNWV |
| NO: 133 | variable | RQAPGQGLEWMGEINPGYGTSRYNQKFKDRVTMTRDT |
| | | SISTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 134 | variable | KPGQAPRLLIYDTSKLASGIPARFSGSGSGTDFTLTI |
| | | SSLEPEDFAVYYCQQWSSNPPTFGQGTKLEIK |

Anti-Mac-1 antibody sequence clone: 28E07-B1H
(Underline is a CDR sequence)

| | | |
|---|---|---|
| SEQ ID | Heavy chain | QVQLVQSGAEVKKPGASVKVSCKASGYSFTDYNMNWV |
| NO: 135 | variable | RQAPGQGLEWMGEINPGYGTSRYNQKFKDRVTLTVDQ |
| | | SITTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 136 | variable | KPGQAPRLLIYDTSKLASGIPARFSGSGSGTDFTLTI |
| | | SSLEPEDFAVYYCQQWSSNPPTFGQGTKLEIK |

Anti-Mac-1 antibody sequence clone: 28E07-B2H
(Underline is a CDR sequence)

| | | |
|---|---|---|
| SEQ ID | Heavy chain | QVQLVQSGAEVKKPGASVKVSCKASGYSFTDYNMNWV |
| NO: 137 | variable | RQAPGQGLEWIGEINPGYGTSRYNQKFKDKATLTVDQ |
| | | SITTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 138 | variable | KPGQAPRLLIYDTSKLASGIPARFSGSGSGTDFTLTI |
| | | SSLEPEDFAVYYCQQWSSNPPTFGQGTKLEIK |

Anti-Mac-1 antibody sequence clone: 28E07-B3H
(Underline is a CDR sequence)

| | | |
|---|---|---|
| SEQ ID | Heavy chain | QVQLVQSGAEVKKPGASVKISCKASGYSFTDYNMNWV |
| NO: 139 | variable | KQAPGQGLEWIGEINPGYGTSRYNQKFKDKATLTVDQ |
| | | SITTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 140 | variable | KPGQAPRLLIYDTSKLASGIPARFSGSGSGTDFTLTI |
| | | SSLEPEDFAVYYCQQWSSNPPTFGQGTKLEIK |

Anti-Mac-1 antibody sequence clone: 28E07-B4H
(Underline is a CDR sequence)

| | | |
|---|---|---|
| SEQ ID | Heavy chain | QVQLVQSGPEVVKPGASVKISCKASGYSFTDYNMNWV |
| NO: 141 | variable | KQANGQSLEWIGEINPGYGTSRYNQKFKDKATLTVDQ |
| | | SITTAYMELNRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 142 | variable | KPGQAPRLLIYDTSKLASGIPARFSGSGSGTDFTLTI |
| | | SSLEPEDFAVYYCQQWSSNPPTFGQGTKLEIK |

Anti-Mac-1 antibody sequence clone: 28E07-HB1
(Underline is a CDR sequence)

| | | |
|---|---|---|
| SEQ ID | Heavy chain | QVQLVQSGAEVKKPGASVKVSCKASGYSFTDYNMNWV |
| NO: 143 | variable | RQAPGQGLEWMGEINPGYGTSRYNQKFKDRVTMTRDT |
| | | SISTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 144 | variable | KPGQAPRRLIYDTSKLASGIPARFSGSGSGTDFTLTI |
| | | SSLEPEDFAVYYCQQWSSNPPTFGQGTKLEIK |

Anti-Mac-1 antibody sequence clone: 28E07-HB2
(Underline is a CDR sequence)

| | | |
|---|---|---|
| SEQ ID | Heavy chain | QVQLVQSGAEVKKPGASVKVSCKASGYSFTDYNMNWV |
| NO: 145 | variable | RQAPGQGLEWMGEINPGYGTSRYNQKFKDRVTMTRDT |
| | | SISTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 146 | variable | KPGQAPRRWIYDTSKLASGIPARFSGSGSGTDYTLTI |
| | | SSLEPEDFAVYYCQQWSSNPPTFGQGTKLEIK |

Anti-Mac-1 antibody sequence clone: 28E07-HB3
(Underline is a CDR sequence)

| | | |
|---|---|---|
| SEQ ID | Heavy chain | QVQLVQSGAEVKKPGASVKVSCKASGYSFTDYNMNWV |
| NO: 147 | variable | RQAPGQGLEWMGEINPGYGTSRYNQKFKDRVTMTRDT |
| | | SISTAYMELSRLRSDDTAVYYCARADVDYGDVMDYWG |
| | | QGTLVTVSS |
| SEQ ID | Light chain | EIELTQSPATLSASPGERVTMSCSASSSVSYMHWYQQ |
| NO: 148 | variable | KPGQAPKRWIYDTSKLASGVPARFSGSGSGTDYTLTI |
| | | SSMEPEDFATYYCQQWSSNPPTFGQGTKLEIK |

Anti-Mac-1 antibody sequence clone: 28E07-HB4
(Underline is a CDR sequence)

| | | |
|---|---|---|
| SEQ ID | Heavy chain | QVQLVQSGAEVKKPGASVKVSCKASGYSFTDYNMNWV |
| NO: 149 | variable | RQAPGQGLEWMGEINPGYGTSRYNQKFKDRVTMTRDT |
| | | SISTAYMELSRLRSDDTAVYYCARADVDYGDVMDYWG |
| | | QGTLVTVSS |
| SEQ ID | Light chain | EIELTQSPATMSASPGERVTMSCSASSSVSYMHWYQQ |
| NO: 150 | variable | KSGQSPKRWIYDTSKLASGVPARFSGSGSGTDYTLTI |
| | | SSMEPEDFATYYCQQWSSNPPTFGQGTKLEIK |

Anti-Mac-1 antibody sequence clone: 28E07-B1B1
(Underline is a CDR sequence)

| | | |
|---|---|---|
| SEQ ID | Heavy chain | QVQLVQSGAEVKKPGASVKVSCKASGYSFTDYNMNWV |
| NO: 151 | variable | RQAPGQGLEWMGEINPGYGTSRYNQKFKDRVTLTVDQ |
| | | SITTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 152 | variable | KPGQAPRRLIYDTSKLASGIPARFSGSGSGTDFTLTI |
| | | SSLEPEDFAVYYCQQWSSNPPTFGQGTKLEIK |

Anti-Mac-1 antibody sequence clone: 28E07-B1B2
(Underline is a CDR sequence)

| | | |
|---|---|---|
| SEQ ID | Heavy chain | QVQLVQSGAEVKKPGASVKVSCKASGYSFTDYNMNWV |
| NO: 153 | variable | RQAPGQGLEWMGEINPGYGTSRYNQKFKDRVTLTVDQ |
| | | SITTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 154 | variable | KPGQAPRRWIYDTSKLASGIPARFSGSGSGTDYTLTI |
| | | SSLEPEDFAVYYCQQWSSNPPTFGQGTKLEIK |

Anti-Mac-1 antibody sequence clone: 28E07-B2B1
(Underline is a CDR sequence)

| | | |
|---|---|---|
| SEQ ID | Heavy chain | QVQLVQSGAEVKKPGASVKVSCKASGYSFTDYNMNWV |
| NO: 155 | variable | RQAPGQGLEWIGEINPGYGTSRYNQKFKDKATLTVDQ |
| | | SITTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 156 | variable | KPGQAPRRLIYDTSKLASGIPARFSGSGSGTDFTLTI |
| | | SSLEPEDFAVYYCQQWSSNPPTFGQGTKLEIK |

Anti-Mac-1 antibody sequence clone: 28E07-B2B2
(Underline is a CDR sequence)

| | | |
|---|---|---|
| SEQ ID | Heavy chain | QVQLVQSGAEVKKPGASVKVSCKASGYSFTDYNMNWV |
| NO: 157 | variable | RQAPGQGLEWIGEINPGYGTSRYNQKFKDKATLTVDQ |
| | | SITTAYMELSRLRSDDTAVYYCARADVDYGDVMDYWG |
| | | QGTLVTVSS |
| SEQ ID | Light chain | EIVLTQSPATLSLSPGERATLSCSASSSVSYMHWYQQ |
| NO: 158 | variable | KPGQAPRRWIYDTSKLASGIPARFSGSGSGTDYTLTI |
| | | SSLEPEDFAVYYCQQWSSNPPTFGQGTKLEIK |

TABLE II
The anti-Mac-1 antibodies selectively bind to different statuses of human Mac-1.
HEK293/Mac-1 cells (clone1-4) were incubated in PBS (Mock), or PBS/MnCl₂ (Mn²⁺).
Binding of isotype control IgG, the CD11b specific mAb (ICRF44), the CD11b
activation-sensing mAb (CBRM1/5), or the screened anti-Mac-1 antibody was
detected using flow cytometry.

| Clone ID | Mock MFI | Mn²⁺ MFI | Mock/Mn²⁺ Ratio |
|---|---|---|---|
| DF4M-31 | 4634.09 | 13002.09 | 0.36 |
| 24H01 | 9943.78 | 23932.79 | 0.42 |
| 25A04 | 39556.31 | 65804.75 | 0.60 |
| DF3M-5 | 34594.39 | 52465.64 | 0.66 |
| DF4-22 | 24413.75 | 35186.45 | 0.69 |
| 25A06 | 28469.57 | 39371.88 | 0.72 |
| CBRM1/5 | 142914.38 | 181344.86 | 0.79 |
| 24G1 | 33937.61 | 41246.16 | 0.82 |
| 27D06 | 15020.65 | 16648.00 | 0.90 |
| 27A04 | 36111.10 | 39843.69 | 0.91 |
| H4L2 | 41612.7 | 45713.69 | 0.91 |
| 28E07 | 53399.63 | 56373.98 | 0.95 |
| 28E07-HH | 1030000 | 1020000 | 1.01 |
| m2396 | 107729.80 | 107187.79 | 1.01 |
| ICRF44 | 805181.31 | 800850.50 | 1.01 |
| Isotype | 940.56 | 935.35 | 1.01 |
| 27G04 | 43141.59 | 41085.59 | 1.05 |
| 24G09 | 29488.77 | 26908.73 | 1.10 |
| 27E12 | 56805.96 | 50812.85 | 1.12 |
| DF4-25 | 35677.02 | 30475.75 | 1.17 |
| 24F9 | 39762.61 | 33933.75 | 1.17 |
| 24F08 | 50004.22 | 42113.35 | 1.19 |
| 24G05 | 55897.95 | 44758.03 | 1.25 |
| 27A06 | 64572.24 | 47964.83 | 1.35 |
| DF3M-32 | 35513.05 | 25778.02 | 1.38 |
| 28A12 | 55233.79 | 37255.65 | 1.48 |
| 28D06 | 51817.07 | 30600.69 | 1.69 |

TABLE III
The anti-Mac-1 antibodies can serve as agonist to
enhance the conformational change of Mac-1.
HEK293/Mac-1 cells (clone1-4) were incubated
with 10 μg/ml anti-Mac-1 antibodies under the PBS
(Mock) condition. Binding of KIM127 or m24 was
detected using flow cytometry.

| | KIM127 (Expression | m24 (Expression |
|---|---|---|
| Clone | relative to IgG4) | relative to IgG4) |
| DF3M-5 | 2.49 | 28.37 |
| 24G05 | 2.22 | 24.99 |
| 24G1 | 2.23 | 24.68 |
| DF4-25 | 2.23 | 24.00 |
| DF4M-9 | 2.17 | 22.08 |
| 24F08 | 2.17 | 21.23 |
| 24F9 | 2.05 | 19.13 |
| DF3M-32 | 1.93 | 17.89 |
| 25A06 | 1.92 | 16.82 |
| DF4-22 | 1.82 | 15.66 |
| 25A04 | 1.58 | 9.33 |
| DF4M-45 | 1.91 | 8.63 |
| DF4M-31 | 1.13 | 3.77 |
| 24H01 | 1.05 | 1.92 |
| 27D06 | 0.99 | 1.20 |
| IgG4 | 1 | 1 |
| 27G04 | 1.11 | 1.00 |
| m2396 | 1.09 | 0.88 |
| 28A12 | 1.02 | 0.88 |
| 27A06 | 1.04 | 0.88 |
| 28E07-HH | 0.90 | 0.83 |
| 27B10 | 1.04 | 0.81 |
| 27A04 | 1.05 | 0.78 |
| 24G09 | 1.01 | 0.72 |
| M1/70 | 0.94 | 0.71 |
| ICRF44 | 0.94 | 0.66 |
| 28D06 | 0.99 | 0.65 |
| 27E12 | 1.00 | 0.64 |
| 28E07 | 0.98 | 0.63 |
| 28G06 | 1.16 | 0.58 |
| H4L2 | 1.11 | 0.44 |

TABLE IV
The anti-Mac-1 antibody can serve as
an antagonist to reduce the conformational
change of Mac-1. HEK293/Mac-1 cells
(clone1-4) were incubated with 10 μg/ml
anti-Mac-1 antibodies under the PBS/MnCl₂
(Mn²⁺) condition. Binding of KIM127 or
m24 was detected using flow cytometry.

| | KIM127 (Expression | m24 (Expression |
|---|---|---|
| Clone | relative to IgG4) | relative to IgG4) |
| H4L2 | 0.63 | 0.23 |
| 28G06 | 0.65 | 0.45 |
| 28D06 | 0.69 | 0.62 |
| 27B10 | 0.71 | 0.65 |
| ICRF44 | 0.75 | 0.66 |
| 28E07-HH | 0.87 | 0.75 |
| M1/70 | 0.84 | 0.80 |
| m2396 | 0.81 | 0.83 |
| 27E12 | 0.71 | 0.85 |
| 28E07 | 0.65 | 0.85 |
| 27A04 | 0.70 | 0.88 |
| 27A06 | 0.85 | 0.88 |
| 24G09 | 0.89 | 1.00 |
| IgG4 | 1 | 1 |
| 28A12 | 0.87 | 1.07 |
| 27G04 | 0.89 | 1.14 |
| 27D06 | 1.05 | 1.38 |
| DF4M-45 | 1.59 | 1.60 |
| 25A04 | 1.34 | 2.11 |
| 24H01 | 1.49 | 2.36 |
| DF4M-9 | 1.68 | 3.35 |
| 25A06 | 1.74 | 3.50 |
| 24G05 | 1.64 | 3.85 |
| DF4M-31 | 1.48 | 3.87 |
| 24F08 | 1.73 | 4.01 |
| 24F9 | 1.71 | 4.01 |
| 24G1 | 1.75 | 4.02 |
| DF4-25 | 1.75 | 4.34 |
| DF4-22 | 1.64 | 4.37 |
| DF3M-5 | 1.68 | 4.40 |
| DF3M-32 | 1.63 | 4.48 |

TABLE V
Tumor growth inhibition (TGI, %)

| | Group | |
|---|---|---|
| | m2396 | 28E07-HH |
| | 10 mg/kg i.p. | 10 mg/kg i.p. |
| Days | Q3D * 10 doses | Q3D * 10 doses |
| 0 | 0.35 | 1.29 |
| 3 | 8.97 | 12.43 |
| 7 | −52.71 | 2.03 |
| 10 | −3.48 | 28.57 |
| 14 | 28.61 | 38.54 |
| 17 | 0.35 | 1.29 |
| 21 | 33.46 | 33.27 |
| 24 | 30.61 | 40.08 |
| 28 | 20.64 | 35.99 |
| 31 | 20.65 | 36.81 |
| 35 | 23.59 | 35.93 |

TABLE VI
Primer and probe sequences used in digital PCR

| SEQ ID NO | Gene name | Sequence |
|---|---|---|
| 555 | 2-LTR forward primer | 5′-CTAACTAGGGAACCCACTGCT-3′ |
| 556 | 2-LTR reverse primer | 5′-GTAGTTCTGCCAATCAGGGAAG-3′ |
| 557 | 2-LTR probe | 5′-/56-FAM/AGCCTCAAT/ZEN/ |
| | | AAAGCTTGCCTTGAGTGC/3IABkFQ/-3′ |
| 558 | RPP30 forward primer | 5′-AGATTTGGACCTGCGAGCG-3′ |
| 559 | RPP30 reverse primer | 5′-GAGCGGCTGTCTCCACAAGT-3′ |
| 560 | RPP30 probe | 5′-/56-FAM/ |
| | | TTCTGACCT/ZEN/GAAGGCTCTGCGCG/ |
| | | 3IABkFQ/-3′ |

TABLE VII
Heavy-chain CDR region sequences and light-chain CDR region
sequences of SEQ ID NO: 159 through SEQ ID NO: 554

| | | |
|---|---|---|
| SEQ ID Number | Description | Sequence |

Anti-Mac-1 antibody CDR sequence clone: 24F08

| | | |
|---|---|---|
| SEQ ID NO: 159 | CDR-H1 | GFTFSSYWMS |
| SEQ ID NO: 160 | CDR-H2 | IINYSGREADYADSVKG |
| SEQ ID NO: 161 | CDR-H3 | DGSYVGQAHEAFDI |
| SEQ ID NO: 162 | CDR-L1 | RASQSISKYLA |
| SEQ ID NO: 163 | CDR-L2 | GTSNLQS |
| SEQ ID NO: 164 | CDR-L3 | QQSRSWPLT |

Anti-Mac-1 antibody CDR sequence clone: 24F09

| | | |
|---|---|---|
| SEQ ID NO: 165 | CDR-H1 | GFTFSSAWMS |
| SEQ ID NO: 166 | CDR-H2 | TIYWSGSEINYADSVKG |
| SEQ ID NO: 167 | CDR-H3 | SFASGESAMDV |
| SEQ ID NO: 168 | CDR-L1 | RASQSISNYLA |
| SEQ ID NO: 169 | CDR-L2 | DASNLQS |
| SEQ ID NO: 170 | CDR-L3 | QQYYSSPPT |

Anti-Mac-1 antibody CDR sequence clone: 24F11

| | | |
|---|---|---|
| SEQ ID NO: 171 | CDR-H1 | GFTFSTSWMHW |
| SEQ ID NO: 172 | CDR-H2 | IINSGGGEAYYADSVKG |
| SEQ ID NO: 173 | CDR-H3 | GDAAFDY |
| SEQ ID NO: 174 | CDR-L1 | RASQLIRKKLA |
| SEQ ID NO: 175 | CDR-L2 | AASTLQS |
| SEQ ID NO: 176 | CDR-L3 | MQSGSPQYT |

Anti-Mac-1 antibody CDR sequence clone: 24F12

| | | |
|---|---|---|
| SEQ ID NO: 177 | CDR-H1 | GFTFTNYWMG |
| SEQ ID NO: 178 | CDR-H2 | IIISDGGEIIYADSVKG |
| SEQ ID NO: 179 | CDR-H3 | IHAGTGSSADY |
| SEQ ID NO: 180 | CDR-L1 | RASQSIYNYLN |
| SEQ ID NO: 181 | CDR-L2 | DASTLQS |
| SEQ ID NO: 182 | CDR-L3 | QQYYSYPWT |

Anti-Mac-1 antibody CDR sequence clone: 24G01

| | | |
|---|---|---|
| SEQ ID NO: 183 | CDR-H1 | GFTFRTFGMN |
| SEQ ID NO: 184 | CDR-H2 | GIVPSGSEIDYADSVKGR |
| SEQ ID NO: 185 | CDR-H3 | DHSHYTGPFDV |
| SEQ ID NO: 186 | CDR-L1 | RASQSIYSYLN |
| SEQ ID NO: 187 | CDR-L2 | GASILQY |
| SEQ ID NO: 188 | CDR-L3 | HQSNSSPGT |

Anti-Mac-1 antibody CDR sequence clone: 24G05

| | | |
|---|---|---|
| SEQ ID NO: 189 | CDR-H1 | GFTFTSYWMT |
| SEQ ID NO: 190 | CDR-H2 | TIVGGGGEADYADSVKG |
| SEQ ID NO: 191 | CDR-H3 | DYVADNHGAMDY |
| SEQ ID NO: 192 | CDR-L1 | RASQGLSSYLN |
| SEQ ID NO: 193 | CDR-L2 | GMSTLQS |
| SEQ ID NO: 194 | CDR-L3 | MQYYHWPYT |

Anti-Mac-1 antibody CDR sequence clone: 24G07

| | | |
|---|---|---|
| SEQ ID NO: 195 | CDR-H1 | GFTFSDYIMH |
| SEQ ID NO: 196 | CDR-H2 | AISPSGSEIYYADSVKG |
| SEQ ID NO: 197 | CDR-H3 | NAWDNNWVREYGMDY |
| SEQ ID NO: 198 | CDR-L1 | RASQSGNNNLA |
| SEQ ID NO: 199 | CDR-L2 | GASTLQS |
| SEQ ID NO: 200 | CDR-L3 | MQSNSYPLT |

Anti-Mac-1 antibody CDR sequence clone: 24G08

| | | |
|---|---|---|
| SEQ ID NO: 201 | CDR-H1 | GFTFSDYKIH |
| SEQ ID NO: 202 | CDR-H2 | IYADSVKG |
| SEQ ID NO: 203 | CDR-H3 | SSYGEGYAFDY |
| SEQ ID NO: 204 | CDR-L1 | RASQDVDSYLN |
| SEQ ID NO: 205 | CDR-L2 | DAISLQS |
| SEQ ID NO: 206 | CDR-L3 | QQYYSLPLT |

Anti-Mac-1 antibody CDR sequence clone: 24G09

| | | |
|---|---|---|
| SEQ ID NO: 207 | CDR-H1 | GFTFSDYAIG |
| SEQ ID NO: 208 | CDR-H2 | TIYWSGSNAYYADSVKG |
| SEQ ID NO: 209 | CDR-H3 | LFTLGYHGFDV |
| SEQ ID NO: 210 | CDR-L1 | RASQSINTYLN |
| SEQ ID NO: 211 | CDR-L2 | DASNLQS |
| SEQ ID NO: 212 | CDR-L3 | QQYDDLPFT |

Anti-Mac-1 antibody CDR sequence clone: 24G10

| | | |
|---|---|---|
| SEQ ID NO: 213 | CDR-H1 | SGFTFSSNSMS |
| SEQ ID NO: 214 | CDR-H2 | AINYSGREIYYADSVKG |
| SEQ ID NO: 215 | CDR-H3 | TDYNTFDY |
| SEQ ID NO: 216 | CDR-L1 | RASQSNSSHLN |
| SEQ ID NO: 217 | CDR-L2 | GVSNLQS |
| SEQ ID NO: 218 | CDR-L3 | QHYGSTPYT |

Anti-Mac-1 antibody CDR sequence clone: 24G11

| | | |
|---|---|---|
| SEQ ID NO: 219 | CDR-H1 | GFTFSDYYMS |
| SEQ ID NO: 220 | CDR-H2 | VISYGGGEAYYADSVKG |
| SEQ ID NO: 221 | CDR-H3 | AMASEYGPWDY |
| SEQ ID NO: 222 | CDR-L1 | RASQSISRHLT |
| SEQ ID NO: 223 | CDR-L2 | GASTLQS |
| SEQ ID NO: 224 | CDR-L3 | QQYHDWPLT |

Anti-Mac-1 antibody CDR sequence clone: 24G12

| | | |
|---|---|---|
| SEQ ID NO: 225 | CDR-H1 | GFTFGDDYMH |
| SEQ ID NO: 226 | CDR-H2 | AINYDGSWKYYADSVKG |
| SEQ ID NO: 227 | CDR-H3 | LSSIDEPPYGPFDV |
| SEQ ID NO: 228 | CDR-L1 | RASQSISTYLA |
| SEQ ID NO: 229 | CDR-L2 | EASSLQS |
| SEQ ID NO: 230 | CDR-L3 | QQYYNFPPT |

Anti-Mac-1 antibody CDR sequence clone: 24H01

| | | |
|---|---|---|
| SEQ ID NO: 231 | CDR-H1 | GFTFNNYYMS |
| SEQ ID NO: 232 | CDR-H2 | IIYYDGSEADYADSVKG |
| SEQ ID NO: 233 | CDR-H3 | NKDIYSVYGMDY |
| SEQ ID NO: 234 | CDR-L1 | RASQSISNYLA |
| SEQ ID NO: 235 | CDR-L2 | ATSNLQS |
| SEQ ID NO: 236 | CDR-L3 | QQANNTPPT |

Anti-Mac-1 antibody CDR sequence clone: 24H02

| | | |
|---|---|---|
| SEQ ID NO: 237 | CDR-H1 | GFTFSDYWMS |
| SEQ ID NO: 238 | CDR-H2 | SIVYGGSEIDYADSVKG |
| SEQ ID NO: 239 | CDR-H3 | VPGYSGTPFDY |
| SEQ ID NO: 240 | CDR-L1 | RASQSVSRYLA |
| SEQ ID NO: 241 | CDR-L2 | DTSSLQS |
| SEQ ID NO: 242 | CDR-L3 | QQSYSFPLT |

Anti-Mac-1 antibody sequence clone: 24H03
(Underline is a CDR sequence)

| | | |
|---|---|---|
| SEQ ID NO: 243 | CDR-H1 | GFTFKDSWMH |
| SEQ ID NO: 244 | CDR-H2 | IISYSGGEAIYADSVKG |
| SEQ ID NO: 245 | CDR-H3 | DSGGSAMGFDI |
| SEQ ID NO: 246 | CDR-L1 | RASQSIHSYLN |
| SEQ ID NO: 247 | CDR-L2 | GASSLQS |
| SEQ ID NO: 248 | CDR-L3 | QQYYRFPYT |

Anti-Mac-1 antibody CDR sequence clone: 25A02

| | | |
|---|---|---|
| SEQ ID NO: 249 | CDR-H1 | GFTFSDWYLH |
| SEQ ID NO: 250 | CDR-H2 | VINGGGSEIIYADSVKG |
| SEQ ID NO: 251 | CDR-H3 | GGDGDGSGFDD |
| SEQ ID NO: 252 | CDR-L1 | RASQSIHSYLN |
| SEQ ID NO: 253 | CDR-L2 | DASNLQS |
| SEQ ID NO: 254 | CDR-L3 | QQSGNYPFT |

Anti-Mac-1 antibody CDR sequence clone: 25A04

| | | |
|---|---|---|
| SEQ ID NO: 255 | CDR-H1 | GFTFSFTAMH |
| SEQ ID NO: 256 | CDR-H2 | AIIYNGSEADYADSVKG |
| SEQ ID NO: 257 | CDR-H3 | ANDYDHGCCDNYAMDY |
| SEQ ID NO: 258 | CDR-L1 | RASQGIGSYLY |
| SEQ ID NO: 259 | CDR-L2 | DASNLQS |
| SEQ ID NO: 260 | CDR-L3 | MQHGGWPLT |

Anti-Mac-1 antibody CDR sequence clone: 25A06

| | | |
|---|---|---|
| SEQ ID NO: 261 | CDR-H1 | GFTFSSYYMS |
| SEQ ID NO: 262 | CDR-H2 | TINYDGSEKDYADSVKG |
| SEQ ID NO: 263 | CDR-H3 | DRLGNYPWFDV |
| SEQ ID NO: 264 | CDR-L1 | RASQSISNYLA |
| SEQ ID NO: 265 | CDR-L2 | DANNLQS |
| SEQ ID NO: 266 | CDR-L3 | QQSYSWPLT |

Anti-Mac-1 antibody CDR sequence clone: 25A09

| | | |
|---|---|---|
| SEQ ID NO: 267 | CDR-H1 | GFTFTEWWMS |
| SEQ ID NO: 268 | CDR-H2 | TISYGGSEAIYADSVKG |
| SEQ ID NO: 269 | CDR-H3 | TSSDRLLFDY |
| SEQ ID NO: 270 | CDR-L1 | RASQSIKSSLA |
| SEQ ID NO: 271 | CDR-L2 | GASTLQS |
| SEQ ID NO: 272 | CDR-L3 | MQTNRHPWT |

Anti-Mac-1 antibody CDR sequence clone: 25A10

| | | |
|---|---|---|
| SEQ ID NO: 273 | CDR-H1 | GFTFSNYNLH |
| SEQ ID NO: 274 | CDR-H2 | TISYSGSEIIYADSVKG |
| SEQ ID NO: 275 | CDR-H3 | EDEYTYYYFDP |
| SEQ ID NO: 276 | CDR-L1 | RASQSVSSYLA |
| SEQ ID NO: 277 | CDR-L2 | DASKLQS |
| SEQ ID NO: 278 | CDR-L3 | KQSYSSPPT |

Anti-Mac-1 antibody sequence clone: 25B03
(Underline is a CDR sequence)

| | | |
|---|---|---|
| SEQ ID NO: 279 | CDR-H1 | GFTFSDYWMH |
| SEQ ID NO: 281 | CDR-H2 | TIIGGGSEAGYADSVKG |
| SEQ ID NO: 281 | CDR-H3 | DRSYGYLGFDI |
| SEQ ID NO: 282 | CDR-L1 | RASQSIRNSLH |
| SEQ ID NO: 283 | CDR-L2 | SAGKLQS |
| SEQ ID NO: 284 | CDR-L3 | QQSNSFPLT |

Anti-Mac-1 antibody CDR sequence clone: 25B04

| | | |
|---|---|---|
| SEQ ID NO: 285 | CDR-H1 | GFTFSTNWMH |
| SEQ ID NO: 286 | CDR-H2 | MISYSGGEAIYADSVKG |
| SEQ ID NO: 287 | CDR-H3 | NWLPYAMDY |
| SEQ ID NO: 288 | CDR-L1 | RASQSIRSYLS |
| SEQ ID NO: 289 | CDR-L2 | SASTLQS |
| SEQ ID NO: 290 | CDR-L3 | QQSYSTPLT |

Anti-Mac-1 antibody CDR sequence clone: 25B01

| | | |
|---|---|---|
| SEQ ID NO: 291 | CDR-H1 | GFTFSSYDVG |
| SEQ ID NO: 292 | CDR-H2 | GIVPSGGNIYYADSVKG |
| SEQ ID NO: 293 | CDR-H3 | HRSYAYYAFDY |
| SEQ ID NO: 294 | CDR-L1 | RASQNVRNYLG |
| SEQ ID NO: 295 | CDR-L2 | DASSLQS |
| SEQ ID NO: 296 | CDR-L3 | QQYGDWPLT |

Anti-Mac-1 antibody CDR sequence clone: DF3-10

| | | |
|---|---|---|
| SEQ ID NO: 297 | CDR-H1 | GFTFTDAYMS |
| SEQ ID NO: 298 | CDR-H2 | TISSYGSSTYYADSVKG |
| SEQ ID NO: 299 | CDR-H3 | PRYIESPVYDY |
| SEQ ID NO: 300 | CDR-L1 | RASQSIAKYLA |
| SEQ ID NO: 301 | CDR-L2 | ETSNLQS |
| SEQ ID NO: 302 | CDR-L3 | QQSSSSPET |

Anti-Mac-1 antibody CDR sequence clone: DF3-28

| | | |
|---|---|---|
| SEQ ID NO: 303 | CDR-H1 | GFTFTNYWMH |
| SEQ ID NO: 304 | CDR-H2 | TIIYDGGETGYADSVKG |
| SEQ ID NO: 305 | CDR-H3 | NSRKSGMDY |
| SEQ ID NO: 306 | CDR-L1 | RASQSIYKYLN |
| SEQ ID NO: 307 | CDR-L2 | DASTLQS |
| SEQ ID NO: 308 | CDR-L3 | MQYYSDPLT |

Anti-Mac-1 antibody CDR sequence clone: DF3-30

| | | |
|---|---|---|
| SEQ ID NO: 309 | CDR-H1 | GFTFSGYAWS |
| SEQ ID NO: 310 | CDR-H2 | MISPAGGSTYYADSVKG |
| SEQ ID NO: 311 | CDR-H3 | DRNAGGDSYYSFDV |
| SEQ ID NO: 312 | CDR-L1 | RASQSINSHLA |
| SEQ ID NO: 313 | CDR-L2 | AAINLQS |
| SEQ ID NO: 314 | CDR-L3 | QQTNHYPLT |

Anti-Mac-1 antibody CDR sequence clone: DF3-32

| | | |
|---|---|---|
| SEQ ID NO: 315 | CDR-H1 | GFTFSSHAMH |
| SEQ ID NO: 316 | CDR-H2 | SILSGGSETNYADSVKG |
| SEQ ID NO: 317 | CDR-H3 | DTYEVTGNLLDY |
| SEQ ID NO: 318 | CDR-L1 | RASQSVWSYLN |
| SEQ ID NO: 319 | CDR-L2 | GASSLQS |
| SEQ ID NO: 320 | CDR-L3 | MQSYSWPFT |

Anti-Mac-1 antibody CDR sequence clone: DF4-16

| | | |
|---|---|---|
| SEQ ID NO: 321 | CDR-H1 | GFTFNEYAMS |
| SEQ ID NO: 322 | CDR-H2 | SIIPDGSETDYADSVKG |
| SEQ ID NO: 323 | CDR-H3 | SLSSSGMHGDI |
| SEQ ID NO: 324 | CDR-L1 | RASQSINNYLN |
| SEQ ID NO: 325 | CDR-L2 | KASTLQS |
| SEQ ID NO: 326 | CDR-L3 | HQYHSPYT |

Anti-Mac-1 antibody CDR sequence clone: DF4-17

| | | |
|---|---|---|
| SEQ ID NO: 327 | CDR-H1 | GFTFSDYGWH |
| SEQ ID NO: 328 | CDR-H2 | IIESDGSGTYYADSVKG |
| SEQ ID NO: 329 | CDR-H3 | NGEVGERGVRDYDYAMDY |
| SEQ ID NO: 330 | CDR-L1 | RASQSINRYLN |
| SEQ ID NO: 331 | CDR-L2 | ATSSLQS |
| SEQ ID NO: 332 | CDR-L3 | QQYGSTPIT |

Anti-Mac-1 antibody CDR sequence clone: DF4-22

| | | |
|---|---|---|
| SEQ ID NO: 333 | CDR-H1 | GFTFTDYNVH |
| SEQ ID NO: 334 | CDR-H2 | GINSSGSETNYADSVKG |
| SEQ ID NO: 335 | CDR-H3 | DSVFKTVGGYDAVMDY |
| SEQ ID NO: 336 | CDR-L1 | RASQSIYNYLA |
| SEQ ID NO: 337 | CDR-L2 | GTSTLQS |
| SEQ ID NO: 338 | CDR-L3 | MQSYSSPTT |

Anti-Mac-1 antibody CDR sequence clone: DF4-25

| | | |
|---|---|---|
| SEQ ID NO: 339 | CDR-H1 | GFTFKDYWLS |
| SEQ ID NO: 340 | CDR-H2 | IINYGGSETYYADSVKG |
| SEQ ID NO: 341 | CDR-H3 | TQTSYVMDY |
| SEQ ID NO: 342 | CDR-L1 | RASQSVRSGLN |
| SEQ ID NO: 343 | CDR-L2 | AASSLQS |
| SEQ ID NO: 344 | CDR-L3 | MQSHSWPLT |

Anti-Mac-1 antibody CDR sequence clone: DF4-26

| | | |
|---|---|---|
| SEQ ID NO: 345 | CDR-H1 | GFTFSSGYMS |
| SEQ ID NO: 346 | CDR-H2 | TISGSGRETNYADSVKG |
| SEQ ID NO: 347 | CDR-H3 | DAWGGDSYFDP |
| SEQ ID NO: 348 | CDR-L1 | RASQSIWSNLS |
| SEQ ID NO: 349 | CDR-L2 | NASSLQS |
| SEQ ID NO: 350 | CDR-L3 | QQYHGTPIT |

Anti-Mac-1 antibody CDR sequence clone: DF4-42

| | | |
|---|---|---|
| SEQ ID NO: 351 | CDR-H1 | GFTFTDYQMS |
| SEQ ID NO: 352 | CDR-H2 | TIIWSGSETNYADSVKG |
| SEQ ID NO: 353 | CDR-H3 | NKTPFDY |
| SEQ ID NO: 354 | CDR-L1 | RASQSIRTHLA |
| SEQ ID NO: 355 | CDR-L2 | DNSNLQS |
| SEQ ID NO: 356 | CDR-L3 | QQYKGSPLT |

Anti-Mac-1 antibody CDR sequence clone: DF3M-1

| | | |
|---|---|---|
| SEQ ID NO: 357 | CDR-H1 | GFTFTSYAMS |
| SEQ ID NO: 358 | CDR-H2 | SISYSGGETDYADSVKG |
| SEQ ID NO: 359 | CDR-H3 | SKGGLYFDY |
| SEQ ID NO: 360 | CDR-L1 | RASQSISSYLA |
| SEQ ID NO: 361 | CDR-L2 | GASSLQS |
| SEQ ID NO: 362 | CDR-L3 | QQYGSTPET |

Anti-Mac-1 antibody CDR sequence clone: DF3M-2

| | | |
|---|---|---|
| SEQ ID NO: 363 | CDR-H1 | GFTFSGYWIH |
| SEQ ID NO: 364 | CDR-H2 | TISYSGDEAYYADSVKG |
| SEQ ID NO: 365 | CDR-H3 | SPSDGDYGFDY |
| SEQ ID NO: 366 | CDR-L1 | RASQSINNYLS |
| SEQ ID NO: 367 | CDR-L2 | DGRILQS |
| SEQ ID NO: 368 | CDR-L3 | MQYLAYPWT |

Anti-Mac-1 antibody sequence clone: DF3M-5
(Underline is a CDR sequence)

| | | |
|---|---|---|
| SEQ ID NO: 369 | CDR-H1 | GFTFGTYDMH |
| SEQ ID NO: 370 | CDR-H2 | MISPSGGDTYYADSVKG |
| SEQ ID NO: 371 | CDR-H3 | DSSGDWYAMAY |
| SEQ ID NO: 372 | CDR-L1 | RASQSIRRYLA |
| SEQ ID NO: 373 | CDR-L2 | GASNLQS |
| SEQ ID NO: 374 | CDR-L3 | QHSSDTPLT |

Anti-Mac-1 antibody CDR sequence clone: DF3M-18

| | | |
|---|---|---|
| SEQ ID NO: 375 | CDR-H1 | GFTFSDSAMG |
| SEQ ID NO: 376 | CDR-H2 | IISYYGSETYYADSVKG |
| SEQ ID NO: 377 | CDR-H3 | NPDGDLSALDY |
| SEQ ID NO: 378 | CDR-L1 | RASQPISSYLN |
| SEQ ID NO: 379 | CDR-L2 | GASSLQS |
| SEQ ID NO: 380 | CDR-L3 | QQRLRSPFT |

Anti-Mac-1 antibody CDR sequence clone: DF3M-19

| | | |
|---|---|---|
| SEQ ID NO: 381 | CDR-H1 | GFTFTSYAMS |
| SEQ ID NO: 382 | CDR-H2 | SINSGGSETNYADSVKG |
| SEQ ID NO: 383 | CDR-H3 | GEYYTDVWPSGFDI |
| SEQ ID NO: 384 | CDR-L1 | RASQSISNPLN |
| SEQ ID NO: 385 | CDR-L2 | DASTLQS |
| SEQ ID NO: 386 | CDR-L3 | QQYGSSPST |

Anti-Mac-1 antibody CDR sequence clone: DF3M-30

| | | |
|---|---|---|
| SEQ ID NO: 387 | CDR-H1 | GFTFSNYEMG |
| SEQ ID NO: 388 | CDR-H2 | IISWSGSETIYADSVKG |
| SEQ ID NO: 389 | CDR-H3 | NGRGDYAFDF |
| SEQ ID NO: 390 | CDR-L1 | RASQSVSNNLA |
| SEQ ID NO: 391 | CDR-L2 | RTTSLQS |
| SEQ ID NO: 392 | CDR-L3 | MQYGSLPST |

Anti-Mac-1 antibody CDR sequence clone: DF3M-36

| | | |
|---|---|---|
| SEQ ID NO: 393 | CDR-H1 | GFTFSDYGMS |
| SEQ ID NO: 394 | CDR-H2 | IISPGGRETYYADSVKG |
| SEQ ID NO: 395 | CDR-H3 | PDGGYYEFDV |
| SEQ ID NO: 396 | CDR-L1 | RASQSISNYLA |
| SEQ ID NO: 397 | CDR-L2 | DASTLQS |
| SEQ ID NO: 398 | CDR-L3 | HQRNSWPPT |

Anti-Mac-1 antibody CDR sequence clone: DF3M-42

| | | |
|---|---|---|
| SEQ ID NO: 399 | CDR-H1 | GFTFSSYHMH |
| SEQ ID NO: 400 | CDR-H2 | AIDSSGRETFYADSVKG |
| SEQ ID NO: 401 | CDR-H3 | GYGDYFDY |
| SEQ ID NO: 402 | CDR-L1 | RASQSGSNYLA |
| SEQ ID NO: 403 | CDR-L2 | AASTLQS |
| SEQ ID NO: 404 | CDR-L3 | QQSGSTPYT |

Anti-Mac-1 antibody CDR sequence clone: DF4M-1

| | | |
|---|---|---|
| SEQ ID NO: 405 | CDR-H1 | GFTFDNYVMG |
| SEQ ID NO: 406 | CDR-H2 | MINYGGSETIYADSVKG |
| SEQ ID NO: 407 | CDR-H3 | SACDYCDFDY |
| SEQ ID NO: 408 | CDR-L1 | RASQVVGSYLN |
| SEQ ID NO: 409 | CDR-L2 | GASTLQS |
| SEQ ID NO: 410 | CDR-L3 | QQYYNYPGT |

Anti-Mac-1 antibody CDR sequence clone: DF4M-3

| | | |
|---|---|---|
| SEQ ID NO: 411 | CDR-H1 | GFTFTTYYMS |
| SEQ ID NO: 412 | CDR-H2 | TIIPSGSETNYADSVKG |
| SEQ ID NO: 413 | CDR-H3 | VPAASEGPMDY |
| SEQ ID NO: 414 | CDR-L1 | RASQSISRNLA |
| SEQ ID NO: 415 | CDR-L2 | DASSLQS |
| SEQ ID NO: 416 | CDR-L3 | QQYYHSPPT |

Anti-Mac-1 antibody CDR sequence clone: DF4M-7-1

| | | |
|---|---|---|
| SEQ ID NO: 417 | CDR-H1 | GFTFDSYAMH |
| SEQ ID NO: 418 | CDR-H2 | SIDGSGRETDYADSVKG |
| SEQ ID NO: 419 | CDR-H3 | DGSEGYAFDP |
| SEQ ID NO: 420 | CDR-L1 | RASQIIRHKLN |
| SEQ ID NO: 421 | CDR-L2 | DASTLQS |
| SEQ ID NO: 422 | CDR-L3 | QQYNSWPIT |

Anti-Mac-1 antibody CDR sequence clone: DF4M-7-4

| | | |
|---|---|---|
| SEQ ID NO: 423 | CDR-H1 | GFTFTSYIMS |
| SEQ ID NO: 424 | CDR-H2 | IISYSGGETYYADSVKG |
| SEQ ID NO: 425 | CDR-H3 | NGINDDSFDY |
| SEQ ID NO: 426 | CDR-L1 | RASQSISNYLN |
| SEQ ID NO: 427 | CDR-L2 | GASSLQS |
| SEQ ID NO: 428 | CDR-L3 | QQRLHWPGT |

Anti-Mac-1 antibody CDR sequence clone: DF4M-9

| | | |
|---|---|---|
| SEQ ID NO: 429 | CDR-H1 | GFTFTNHWMS |
| SEQ ID NO: 430 | CDR-H2 | TIEGSGSETIYADSVKG |
| SEQ ID NO: 431 | CDR-H3 | SSRTLFDY |
| SEQ ID NO: 432 | CDR-L1 | RASQGVYSYLA |
| SEQ ID NO: 433 | CDR-L2 | DASSLQS |
| SEQ ID NO: 434 | CDR-L3 | QQYYHYPPT |

Anti-Mac-1 antibody CDR sequence clone: DF4M-11

| | | |
|---|---|---|
| SEQ ID NO: 435 | CDR-H1 | GFTFSNYYMD |
| SEQ ID NO: 436 | CDR-H2 | SINPWGGNKYYADSVKG |
| SEQ ID NO: 437 | CDR-H3 | TITSKYEDYAMDY |
| SEQ ID NO: 438 | CDR-L1 | RASQSISSYLA |
| SEQ ID NO: 439 | CDR-L2 | LTSNLQS |
| SEQ ID NO: 440 | CDR-L3 | QQTAQNPFT |

Anti-Mac-1 antibody CDR sequence clone: DF4M-17

| | | |
|---|---|---|
| SEQ ID NO: 441 | CDR-H1 | GFTFSDYWVA |
| SEQ ID NO: 442 | CDR-H2 | TISYSGSETEYADSVKG |
| SEQ ID NO: 443 | CDR-H3 | YGGSDYYGFDP |
| SEQ ID NO: 444 | CDR-L1 | RASQSISNNLA |
| SEQ ID NO: 445 | CDR-L2 | ATTTLQS |
| SEQ ID NO: 446 | CDR-L3 | MQSNTPWT |

Anti-Mac-1 antibody CDR sequence clone: DF4M-18

| | | |
|---|---|---|
| SEQ ID NO: 447 | CDR-H1 | GFTFSSYAIS |
| SEQ ID NO: 448 | CDR-H2 | AISSGGSETDYADSVKG |
| SEQ ID NO: 449 | CDR-H3 | GESGYYMAEDV |
| SEQ ID NO: 450 | CDR-L1 | RASQSVSSFLA |
| SEQ ID NO: 451 | CDR-L2 | AASKLQS |
| SEQ ID NO: 452 | CDR-L3 | QQYSVTPIT |

Anti-Mac-1 antibody CDR sequence clone: DF4M-21

| | | |
|---|---|---|
| SEQ ID NO: 453 | CDR-H1 | GFTFSSYAMS |
| SEQ ID NO: 454 | CDR-H2 | AISSYGGETDYADSVKG |
| SEQ ID NO: 455 | CDR-H3 | GDAYSSFVDNPFDI |
| SEQ ID NO: 456 | CDR-L1 | RASQSISNYLA |
| SEQ ID NO: 457 | CDR-L2 | DASTLQS |
| SEQ ID NO: 458 | CDR-L3 | MQYESTPWT |

Anti-Mac-1 antibody CDR sequence clone: DF4M-23

| | | |
|---|---|---|
| SEQ ID NO: 459 | CDR-H1 | GFTFTSYAMS |
| SEQ ID NO: 460 | CDR-H2 | AISPSGSETEYADSVKG |
| SEQ ID NO: 461 | CDR-H3 | GFYNDYIFDL |
| SEQ ID NO: 462 | CDR-L1 | RASQSISSYLN |
| SEQ ID NO: 463 | CDR-L2 | AASSLQS |
| SEQ ID NO: 464 | CDR-L3 | QQYLSTPYT |

Anti-Mac-1 antibody CDR sequence clone: DF4M-30

| | | |
|---|---|---|
| SEQ ID NO: 465 | CDR-H1 | GFTFRNNAMH |
| SEQ ID NO: 466 | CDR-H2 | VINSGGSETYYADSVKG |
| SEQ ID NO: 467 | CDR-H3 | DEPSDEYGMYGFDY |
| SEQ ID NO: 468 | CDR-L1 | RASQSISSYLN |
| SEQ ID NO: 469 | CDR-L2 | KASNLQS |
| SEQ ID NO: 470 | CDR-L3 | VQYSRSPTT |

Anti-Mac-1 antibody CDR sequence clone: DF4M-31

| | | |
|---|---|---|
| SEQ ID NO: 471 | CDR-H1 | GFTFTSATMS |
| SEQ ID NO: 472 | CDR-H2 | IISPGGSETYYADSVKG |
| SEQ ID NO: 473 | CDR-H3 | GGDYPSYYMDP |
| SEQ ID NO: 474 | CDR-L1 | RASQSISNYLA |
| SEQ ID NO: 475 | CDR-L2 | GTSSLQS |
| SEQ ID NO: 476 | CDR-L3 | QQGHQWPWT |

Anti-Mac-1 antibody CDR sequence clone: DF4M-45

| | | |
|---|---|---|
| SEQ ID NO: 477 | CDR-H1 | GFTFTSYYMS |
| SEQ ID NO: 478 | CDR-H2 | TIISDGSETGYADSVKG |
| SEQ ID NO: 479 | CDR-H3 | TNRYGFQFDY |
| SEQ ID NO: 480 | CDR-L1 | RASQGARNGLH |
| SEQ ID NO: 481 | CDR-L2 | DASTLQS |
| SEQ ID NO: 482 | CDR-L3 | QQRYSYPPT |

Anti-Mac-1 antibody CDR sequence clone: 28E07, 28E07-HH,
28E07-B1H, 28E07-B2H, 28E07-B3H, 28E07-B4H, 28E07-HB1,
28E07-HB2, 28E07-HB3, 28E07-HB4, 28E07-B1B1,
28E07-B1B2, 28E07-B2B1, and 28E07-B2B2

| | | |
|---|---|---|
| SEQ ID NO: 483 | CDR-H1 | GYSFTDYNMN |
| SEQ ID NO: 484 | CDR-H2 | EINPGYGTSRYNQKFKD |
| SEQ ID NO: 485 | CDR-H3 | ADVDYGDVMDY |
| SEQ ID NO: 486 | CDR-L1 | SASSSVSYMH |
| SEQ ID NO: 487 | CDR-L2 | DTSKLAS |
| SEQ ID NO: 488 | CDR-L3 | QQWSSNPPT |

Anti-Mac-1 antibody CDR sequence clone: 28A12

| | | |
|---|---|---|
| SEQ ID NO: 489 | CDR-H1 | GYSFTDYNMN |
| SEQ ID NO: 490 | CDR-H2 | EINPGYGTSRYNQKFKD |
| SEQ ID NO: 491 | CDR-H3 | ADVDYGDTMDY |
| SEQ ID NO: 492 | CDR-L1 | SASSSVSDMH |
| SEQ ID NO: 493 | CDR-L2 | DTSKLAS |
| SEQ ID NO: 494 | CDR-L3 | QQWSSNPPT |

Anti-Mac-1 antibody CDR sequence clone: 27G04

| | | |
|---|---|---|
| SEQ ID NO: 495 | CDR-H1 | GYSFTDYNMN |
| SEQ ID NO: 496 | CDR-H2 | VINPNYGTTSYNQKFKG |
| SEQ ID NO: 497 | CDR-H3 | TFDYDDDAFAY |
| SEQ ID NO: 498 | CDR-L1 | SASSSVSDMH |
| SEQ ID NO: 499 | CDR-L2 | DTSKLAS |
| SEQ ID NO: 500 | CDR-L3 | QQWSSNPPT |

Anti-Mac-1 antibody CDR sequence clone: 27A04

| | | |
|---|---|---|
| SEQ ID NO: 501 | CDR-H1 | GYSFTDYNMN |
| SEQ ID NO: 502 | CDR-H2 | VINPNYGTTSYNQKFKG |
| SEQ ID NO: 503 | CDR-H3 | TYDYDGDAFAY |
| SEQ ID NO: 504 | CDR-L1 | SASSSVSYMH |
| SEQ ID NO: 505 | CDR-L2 | DTSKLAS |
| SEQ ID NO: 506 | CDR-L3 | QQWSSNPPT |

Anti-Mac-1 antibody CDR sequence clone: 27A06

| | | |
|---|---|---|
| SEQ ID NO: 507 | CDR-H1 | GYSFTDYNMN |
| SEQ ID NO: 508 | CDR-H2 | EINPNYGTTRHNQKFKG |
| SEQ ID NO: 509 | CDR-H3 | TYDYDEDAFAY |
| SEQ ID NO: 510 | CDR-L1 | SASSSVSYMH |
| SEQ ID NO: 511 | CDR-L2 | DTSKLAS |
| SEQ ID NO: 512 | CDR-L3 | QQWSSNPPT |

Anti-Mac-1 antibody CDR sequence clone: 28G06

| | | |
|---|---|---|
| SEQ ID NO: 513 | CDR-H1 | GYSFTDYNMN |
| SEQ ID NO: 514 | CDR-H2 | IINPNYGTTSYNQKFKG |
| SEQ ID NO: 515 | CDR-H3 | GYDYDESGFAY |
| SEQ ID NO: 516 | CDR-L1 | SASSSVSYMY |
| SEQ ID NO: 517 | CDR-L2 | DTSNLAS |
| SEQ ID NO: 518 | CDR-L3 | QQWSSNPPT |

Anti-Mac-1 antibody CDR sequence clone: 27B10

| | | |
|---|---|---|
| SEQ ID NO: 519 | CDR-H1 | GYSFTDYNMN |
| SEQ ID NO: 520 | CDR-H2 | RINPNFGTTTYNQKFKG |
| SEQ ID NO: 521 | CDR-H3 | GYDYDESGFAY |
| SEQ ID NO: 522 | CDR-L1 | SASSSVSYMY |
| SEQ ID NO: 523 | CDR-L2 | DTSNLAS |
| SEQ ID NO: 524 | CDR-L3 | QQWSSYPPT |

Anti-Mac-1 antibody CDR sequence clone: 27D06

| | | |
|---|---|---|
| SEQ ID NO: 525 | CDR-H1 | GYSFTDYNMN |
| SEQ ID NO: 526 | CDR-H2 | VINPNYGTTSYNQKFKG |
| SEQ ID NO: 527 | CDR-H3 | TYDYDGDAFAY |
| SEQ ID NO:528 | CDR-L1 | RASSSVSSNNLH |
| SEQ ID NO:529 | CDR-L2 | STSNLAT |
| SEQ ID NO: 530 | CDR-L3 | QQWNSNPPT |

Anti-Mac-1 antibody CDR sequence clone: 28D06

| | | |
|---|---|---|
| SEQ ID NO: 531 | CDR-H1 | GYSFTDYNMN |
| SEQ ID NO: 532 | CDR-H2 | EINPNYGTTRYNQKFKG |
| SEQ ID NO: 533 | CDR-H3 | PSIYYDYDDAMDY |
| SEQ ID NO: 534 | CDR-L1 | SASSSVNYMY |
| SEQ ID NO: 535 | CDR-L2 | DTSNLAS |
| SEQ ID NO: 536 | CDR-L3 | QQWITYPPTLT |

Anti-Mac-1 antibody CDR sequence clone: 27E12

| | | |
|---|---|---|
| SEQ ID NO: 537 | CDR-H1 | GYTFTSYWMH |
| SEQ ID NO: 538 | CDR-H2 | AIYPGNSDTSYNQKFKGKA |
| SEQ ID NO: 539 | CDR-H3 | GSYEFAY |
| SEQ ID NO: 540 | CDR-L1 | SVSSSVSYMY |
| SEQ ID NO: 541 | CDR-L2 | DTSNLAS |
| SEQ ID NO: 542 | CDR-L3 | QQWSSNPFT |

Anti-Mac-1 antibody CDR sequence clone: m2396

| | | |
|---|---|---|
| SEQ ID NO: 543 | CDR-H1 | GFSLTSNSIS |
| SEQ ID NO: 544 | CDR-H2 | AIWSGGGTDYNPSLKS |
| SEQ ID NO: 545 | CDR-H3 | RGGYPYYFDY |
| SEQ ID NO: 546 | CDR-L1 | KSSQSLLYSENQENYLA |
| SEQ ID NO: 547 | CDR-L2 | WASTRQS |
| SEQ ID NO: 548 | CDR-L3 | QQYYDTPLT |

Anti-Mac-1 antibody CDR sequence clone: H4L2

| | | |
|---|---|---|
| SEQ ID NO: 549 | CDR-H1 | NYWIN |
| SEQ ID NO: 550 | CDR-H2 | NIYPSDTYINHNQKFKD |
| SEQ ID NO: 551 | CDR-H3 | SAYANYFDY |
| SEQ ID NO: 552 | CDR-L1 | RASQNIGTSIH |
| SEQ ID NO: 553 | CDR-L2 | YASESIS |
| SEQ ID NO: 554 | CDR-L3 | QQSDSWPTLT |

1. An antibody against human Mac-1, wherein the antibody binds to a specific state of human Mac-1 and modulates Th1 and/or Th2 cytokine secretions by TLR-activated immune cells.

2. The antibody according to claim 1, wherein the Th1 cytokine secretion is enhanced to a greater extent than the Th2 cytokine secretion.

3. The antibody according to claim 1, wherein the antibody comprises a heavy-chain variable region sequence having CDR-H1, CDR-H2, and CDR-H3 sequences; and a light-chain variable region sequence having CDR-L1, CDR-L2, and CDR-L3 sequences, wherein
the CDR-H1 has the sequence of SEQ ID NO: 543, 549, 483, 369, 189, 159, 165, 171, 177, 183, 195, 201, 207, 213, 219, 225, 231, 237, 243, 249, 255, 261, 267, 273, 279, 285, 291, 297, 303, 309, 315, 321, 327, 333, 339, 345, 351, 357, 363, 375, 381, 387, 393, 399, 405, 411, 417, 423, 429, 435, 441, 447, 453, 459, 465, 471, 477, 489, 495, 501, 507, 513, 519, 525, 531, or 537;
the CDR-H2 has the sequence of SEQ ID NO: 544, 550, 484, 370, 190, 160, 166, 172, 178, 184, 196, 202, 208, 214, 220, 226, 232, 238, 244, 250, 256, 262, 268, 274, 280, 286, 292, 298, 304, 310, 316, 322, 328, 334, 340, 346, 352, 358, 364, 376, 382, 388, 394, 400, 406, 412, 418, 424, 430, 436, 442, 448, 454, 460, 466, 472, 478, 490, 496, 502, 508, 514, 520, 526, 532, or 538;
the CDR-H3 has the sequence of SEQ ID NO: 545, 551, 485, 371, 191, 161, 167, 173, 179, 185, 197, 203, 209, 215, 221, 227, 233, 239, 245, 251, 257, 263, 269, 275, 281, 287, 293, 299, 305, 311, 317, 323, 329, 335, 341, 347, 353, 359, 365, 377, 383, 389, 395, 401, 407, 413, 419, 425, 431, 437, 443, 449, 455, 461, 467, 473, 479, 491, 497, 503, 509, 515, 521, 527, 533, or 539;
the CDR-L1 has the sequence of SEQ ID NO: 546, 552, 486, 372, 192, 162, 168, 174, 180, 186, 198, 204, 210, 216, 222, 228, 234, 240, 246, 252, 258, 264, 270, 276, 282, 288, 294, 300, 306, 312, 318, 324, 330, 336, 342, 348, 354, 360, 366, 378, 384, 390, 396, 402, 408, 414, 420, 426, 432, 438, 444, 450, 456, 462, 468, 474, 480, 492, 498, 504, 510, 516, 522, 528, 534, or 540;
the CDR-L2 has the sequence of SEQ ID NO: 547, 553, 487, 373, 193, 163, 169, 175, 181, 187, 199, 205, 211, 217, 223, 229, 235, 241, 247, 253, 259, 265, 271, 277, 283, 289, 295, 301, 307, 313, 319, 325, 331, 337, 343, 349, 355, 361, 367, 379, 385, 391, 397, 403, 409, 415, 421, 427, 433, 439, 445, 451, 457, 463, 469, 475, 481, 493, 499, 505, 511, 517, 523, 529, 535, or 541; and
the CDR-L3 has the sequence of SEQ ID NO: 548, 554, 488, 374, 194, 164, 170, 176, 182, 188, 200, 206, 212, 218, 224, 230, 236, 242, 248, 254, 260, 266, 272, 278, 284, 290, 296, 302, 308, 314, 320, 326, 332, 338, 344, 350, 356, 362, 368, 380, 386, 392, 398, 404, 410, 416, 422, 428, 434, 440, 446, 452, 458, 464, 470, 476, 482, 494, 500, 506, 512, 518, 524, 530, 536, or 542.

4. The antibody against human Mac-1 according to claim 1, wherein the CDR-H1, CDRH2, CDR-H3, CDR-L1, CDR-L2, and CDR-L3 have the sequences of SEQ ID NO: 159-164, or SEQ ID NO: 165-170, or SEQ ID NO: 171-176, or SEQ ID NO: 177-182, or SEQ ID NO: 183-188, or SEQ ID NO: 189-194, or SEQ ID NO: 195-200, or SEQ ID NO: 201-206, or SEQ ID NO: 207-212, or SEQ ID NO: 213-218, or SEQ ID NO: 219-224, or SEQ ID NO: 225-230, or SEQ ID NO: 231-236, or SEQ ID NO: 237-242, or SEQ ID NO: 243-248, or SEQ ID NO: 249-254, or SEQ ID NO: 255-260, or SEQ ID NO: 261-266, or SEQ ID NO: 267-272, or SEQ ID NO: 273-278, or SEQ ID NO: 279-284, or SEQ ID NO: 285-290, or SEQ ID NO: 291-296, or SEQ ID NO: 297-302, or SEQ ID NO: 303-308, or SEQ ID NO: 309-314, or SEQ ID NO: 315-320, or SEQ ID NO: 321-326, or SEQ ID NO: 327-332, or SEQ ID NO: 333-338, or SEQ ID NO: 339-344, or SEQ ID NO: 345-350, or SEQ ID NO: 351-356, or SEQ ID NO: 357-362, or SEQ ID NO: 363-368, or SEQ ID NO: 369-374, or SEQ ID NO: 375-380, or SEQ ID NO: 381-386, or SEQ ID NO: 387-392, or SEQ ID NO: 393-398, or SEQ ID NO: 399-404, or SEQ ID NO: 405-410, or SEQ ID NO: 411-416, or SEQ ID NO: 417-422, or SEQ ID NO: 423-428, or SEQ ID NO: 429-434, or SEQ ID NO: 435-440, or SEQ ID NO: 441-446, or SEQ ID NO: 447-452, or SEQ ID NO: 453-458, or SEQ ID NO: 459-464, or SEQ ID NO: 465-470, or SEQ ID NO: 471-476, or SEQ ID NO: 477-482, or SEQ ID NO: 483-488, or SEQ ID NO: 489-494, or SEQ ID NO: 495-500, or SEQ ID NO: 501-506, or SEQ ID NO: 507-512, or SEQ ID NO: 513-518, or SEQ ID NO: 519-524, or SEQ ID NO: 525-530, or SEQ ID NO: 531-536, or SEQ ID NO: 537-542, or SEQ ID NO: 543-548, or SEQ ID NO: 549-554.

5. The antibody against human Mac-1 according to claim 1, wherein the CDR-H1, CDRH2, CDR-H3, CDR-L1, CDR-L2, and CDR-L3 have the sequences of SEQ ID NO: 543-548, or SEQ ID NO: 549-554, or SEQ ID NO: 483-488, or SEQ ID NO: 369-374, or SEQ ID NO: 189-194.

6. The antibody against human Mac-1 according to claim 1, wherein the heavy-chain variable region sequence is SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, or 157; and the light-chain variable region sequence is SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158.

7. The antibody against human Mac-1 according to claim 1, wherein the heavy-chain variable region sequence and the light-chain variable region sequence have the following sequence pair: SEQ ID NO:1 and 2, or SEQ ID NO:3 and 4, or SEQ ID NO:5 and 6, or SEQ ID NO:7 and 8, or SEQ ID NO:9 and 10, or SEQ ID NO:11 and 12, or SEQ ID NO:13 and 14, or SEQ ID NO:15 and 16, or SEQ ID NO:17 and 18, or SEQ ID NO:19 and 20, or SEQ ID NO:21 and 22, or SEQ ID NO:23 and 24, or SEQ ID NO:25 and 26, or SEQ ID NO:27 and 28, or SEQ ID NO:29 and 30, or SEQ ID NO:31 and 32, or SEQ ID NO:33 and 34, or SEQ ID NO:35 and 36, or SEQ ID NO:37 and 38, or SEQ ID NO:39 and 40, or SEQ ID NO:41 and 42, or SEQ ID NO:43 and 44, or SEQ ID NO:45 and 46, or SEQ ID NO:47 and 48, or SEQ ID NO:49 and 50, or SEQ ID NO:51 and 52, or SEQ ID NO:53 and 54, or SEQ ID NO:55 and 56, or SEQ ID NO:57 and 58, or SEQ ID NO:59 and 60, or SEQ ID NO:61 and 62, or SEQ ID NO:63 and 64, or SEQ ID NO:65 and 66, or SEQ ID NO:67 and 68, or SEQ ID NO:69 and 70, or SEQ ID NO:71 and 72, or SEQ ID NO:73 and 74, or SEQ ID NO:75 and 76, or SEQ ID NO:77 and 78, or SEQ ID NO:79 and 80, or SEQ ID NO:81 and 82, or SEQ ID NO:83 and 842, or SEQ ID NO:85 and 86, or SEQ ID NO:87 and 88, or SEQ ID NO:89 and 90, or SEQ ID NO:91 and 92, or SEQ ID NO:93 and 94, or SEQ ID NO:95 and 96, or SEQ ID NO:97 and 98, or SEQ ID NO:99 and 100, or SEQ ID NO:101 and 102, or SEQ ID NO:103 and 104, or SEQ ID NO:105 and 106, or SEQ ID NO:107 and 108, or SEQ ID NO:109 and 110, or SEQ ID NO:111 and 112, or SEQ ID NO:113 and 114, or SEQ ID NO:115 and 116, or SEQ ID NO:117 and 118, or SEQ ID NO:119 and 120, or SEQ ID NO:121 and 122, or SEQ ID NO:123 and 124, or SEQ ID NO:125 and 126, or SEQ ID NO:127 and 128, or SEQ ID NO:129 and 130, or SEQ ID NO:131 and 132, or SEQ ID NO:133 and 134, or SEQ ID NO:135 and 136, or SEQ ID NO:137 and 138, or SEQ ID NO:139 and 140, or SEQ ID NO:141 and 142, or SEQ ID NO:143 and 144, or SEQ ID NO:145 and 146, or SEQ ID NO:147 and 148, or SEQ ID NO:149 and 150, or SEQ ID NO:151 and 152, or SEQ ID NO:153 and 154, or SEQ ID NO:155 and 156, or SEQ ID NO:157 and 158.

8. The antibody against human Mac-1 according to claim 1, wherein the heavy-chain variable region sequence and the light-chain variable region sequence have the following sequence pair: SEQ ID NO:129 and 130, or SEQ ID NO:131 and 132, or SEQ ID NO:133 and 134, or SEQ ID NO:71 and 72, or SEQ ID NO:11 and 12.

9. A composition wherein the composition comprises the antibody according to claim 1.

10. (canceled)

11. The composition of claim 9, wherein the composition further comprises a carrier.

12. The composition of claim 11, wherein the composition is a pharmaceutical composition and the carrier is a pharmaceutically acceptable carrier.

13. A method of treating a disease or disorder associated with acute or chronic inflammation or cancer, the method comprising administering to a subject in need thereof an effective amount of the pharmaceutical composition of claim 12.
